SEQUENCE LISTING (99 sequences)

SEQ ID NO: 1 - protein, 16 aa, Artificial Sequence
  Description of Artificial Sequence Synthetic peptide
RSSQSLVHRNGNTYLH

SEQ ID NO: 2 - protein, 7 aa, Artificial Sequence
  Description of Artificial Sequence Synthetic peptide
TVSNRFS

SEQ ID NO: 3 - protein, 9 aa, Artificial Sequence
  Description of Artificial Sequence Synthetic peptide
SQSSHVPPT

SEQ ID NO: 4 - protein, 5 aa, Artificial Sequence
  Description of Artificial Sequence Synthetic peptide
NYGVN

SEQ ID NO: 5 - protein, 17 aa, Artificial Sequence
  Description of Artificial Sequence Synthetic peptide
WINPNTGEPTFDDDFKG

SEQ ID NO: 6 - protein, 11 aa, Artificial Sequence
  Description of Artificial Sequence Synthetic peptide
SRGKNEAWFAY

SEQ ID NO: 7 - protein, 5 aa, Artificial Sequence
  Description of Artificial Sequence Synthetic peptide
NYGMN

SEQ ID NO: 8 - protein, 17 aa, Artificial Sequence
  Description of Artificial Sequence Synthetic peptide
WINTYTREPTYADDFKG

SEQ ID NO: 9 - protein, 12 aa, Artificial Sequence
  Description of Artificial Sequence Synthetic peptide
DITAVVPTGFDY

SEQ ID NO: 10 - protein, 11 aa, Artificial Sequence
  Description of Artificial Sequence Synthetic peptide
RASENIYSNLA

SEQ ID NO: 11 - protein, 7 aa, Artificial Sequence
  Description of Artificial Sequence Synthetic peptide
AASNLAD

SEQ ID NO: 12 - protein, 9 aa, Artificial Sequence
  Description of Artificial Sequence Synthetic peptide
QHFWTTPWA

SEQ ID NO: 13 - DNA, 21 bp, Artificial Sequence
  Description of Artificial Sequence Synthetic oligonucleotide
aatgcggcggtggtgacagta

SEQ ID NO: 14 - DNA, 21 bp, Artificial Sequence
  Description of Artificial Sequence Synthetic oligonucleotide
aagctcagcacacagaaagac

SEQ ID NO: 15 - DNA, 21 bp, Artificial Sequence
  Description of Artificial Sequence Synthetic oligonucleotide
uaaaaucuuccugcccacctt

SEQ ID NO: 16 - DNA, 21 bp, Artificial Sequence
  Description of Artificial Sequence Synthetic oligonucleotide
ggaagcuguuggcugaaaatt

SEQ ID NO: 17 - RNA, 21 nt, Artificial Sequence
  Description of Artificial Sequence Synthetic oligonucleotide
aagaccagccucuuugcccag

SEQ ID NO: 18 - DNA, 19 bp, Artificial Sequence
  Description of Artificial Sequence Synthetic oligonucleotide
ggaccaggcagaaaacgag

SEQ ID NO: 19 - RNA, 17 nt, Artificial Sequence
  Description of Artificial Sequence Synthetic oligonucleotide
cuaucaggaugacgcgg

SEQ ID NO: 20 - RNA, 21 nt, Artificial Sequence
  Description of Artificial Sequence Synthetic oligonucleotide
ugacacaggcaggcuugacuu

SEQ ID NO: 21 - DNA, 19 bp, Artificial Sequence
  Description of Artificial Sequence Synthetic oligonucleotide
ggtgaagaagggcgtccaa

SEQ ID NO: 22 - DNA, 60 bp, Artificial Sequence
  Description of Artificial Sequence Synthetic oligonucleotide
gatccgttggagctgttggcgtagttcaagagactcgccaacagctccaacttttggaaa

SEQ ID NO: 23 - DNA, 20 bp, Artificial Sequence
  Description of Artificial Sequence Synthetic oligonucleotide
aggtggtgttaacagcagag

SEQ ID NO: 24 - DNA, 21 bp, Artificial Sequence
  Description of Artificial Sequence Synthetic oligonucleotide
aaggtggagcaagcggtggag

SEQ ID NO: 25 - DNA, 21 bp, Artificial Sequence
  Description of Artificial Sequence Synthetic oligonucleotide
aaggagttgaaggccgacaaa

SEQ ID NO: 26 - DNA, 21 bp, Artificial Sequence
  Description of Artificial Sequence Synthetic oligonucleotide
uauggagcugcagaggaugtt

SEQ ID NO: 27 - DNA, 49 bp, Artificial Sequence
  Description of Artificial Sequence Synthetic oligonucleotide
tttgaatatctgtgctgagaacacagttctcagcacagatattcttttt

SEQ ID NO: 28 - DNA, 29 bp, Artificial Sequence
  Description of Artificial Sequence Synthetic oligonucleotide
aatgagaaaagcaaaaggtgccctgtctc

SEQ ID NO: 29 - RNA, 21 nt, Artificial Sequence
  Description of Artificial Sequence Synthetic oligonucleotide
aaucaucaucaagaaagggca

SEQ ID NO: 30 - DNA, 21 bp, Artificial Sequence
  Description of Artificial Sequence Synthetic oligonucleotide
augacugucaggauguugctt

SEQ ID NO: 31 - RNA, 21 nt, Artificial Sequence
  Description of Artificial Sequence Synthetic oligonucleotide
gaacgaauccugaagacaucu

SEQ ID NO: 32 - DNA, 29 bp, Artificial Sequence
  Description of Artificial Sequence Synthetic oligonucleotide
aagcctggctacagcaatatgcctgtctc

SEQ ID NO: 33 - DNA, 21 bp, Artificial Sequence
  Description of Artificial Sequence Synthetic oligonucleotide
ugaccaucaccgaguuuautt

SEQ ID NO: 34 - DNA, 21 bp, Artificial Sequence
  Description of Artificial Sequence Synthetic oligonucleotide
aagtcggacgcaacagagaaa

SEQ ID NO: 35 - DNA, 21 bp, Artificial Sequence
  Description of Artificial Sequence Synthetic oligonucleotide
cuaccuuucuacggacgugtt

SEQ ID NO: 36 - DNA, 21 bp, Artificial Sequence
  Description of Artificial Sequence Synthetic oligonucleotide
ctgcctaaggcggatttgaat

SEQ ID NO: 37 - DNA, 21 bp, Artificial Sequence
  Description of Artificial Sequence Synthetic oligonucleotide
ttauuccuucuucgggaaguc

SEQ ID NO: 38 - DNA, 21 bp, Artificial Sequence
  Description of Artificial Sequence Synthetic oligonucleotide
aaccttctggaacccgcccac

SEQ ID NO: 39 - DNA, 19 bp, Artificial Sequence
  Description of Artificial Sequence Synthetic oligonucleotide
gagcatcttcgagcaagaa

SEQ ID NO: 40 - DNA, 19 bp, Artificial Sequence
  Description of Artificial Sequence Synthetic oligonucleotide
catgtggcaccgtttgcct

SEQ ID NO: 41 - DNA, 21 bp, Artificial Sequence
  Description of Artificial Sequence Synthetic oligonucleotide
aactaccagaaaggtatacct

SEQ ID NO: 42 - DNA, 21 bp, Artificial Sequence
  Description of Artificial Sequence Synthetic oligonucleotide
ucacaguguccuuuauguatt

SEQ ID NO: 43 - DNA, 21 bp, Artificial Sequence
  Description of Artificial Sequence Synthetic oligonucleotide
gcaugaaccggaggcccautt

SEQ ID NO: 44 - DNA, 19 bp, Artificial Sequence
  Description of Artificial Sequence Synthetic oligonucleotide
ccggacagttccatgtata

SEQ ID NO: 45 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 46 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 47 - protein, 17 aa, Artificial Sequence
  Description of Artificial Sequence Synthetic peptide
QIEYLAKQIVDNAIQQA

SEQ ID NO: 48 - protein, 21 aa, Artificial Sequence
  Description of Artificial Sequence Synthetic peptide
CGQIEYLAKQIVDNAIQQAGC

SEQ ID NO: 49 - protein, 50 aa, Homo sapiens
SLRECELYVQKHNIQALLKDSIVQLCTARPERPMAFLREYFERLEKEEAK

SEQ ID NO: 50 - protein, 55 aa, Homo sapiens
MSCGGSLRECELYVQKHNIQALLKDSIVQLCTARPERPMAFLREYFERLEKEEAK

SEQ ID NO: 51 - protein, 23 aa, Homo sapiens
CGFEELAWKIAKMIWSDVFQQGC

SEQ ID NO: 52 - protein, 55 aa, Artificial Sequence
  Description of Artificial Sequence Synthetic polypeptide
GSGGGGSGGGGSHIQIPPGLTELLQGYTVEVLRQQPPDLVEFAVEYFTRLREARA

SEQ ID NO: 53 - protein, 29 aa, Artificial Sequence
  Description of Artificial Sequence Synthetic peptide
GSGGGGSGGGGSQIEYLAKQIVDNAIQQA

SEQ ID NO: 54 - protein, 51 aa, Homo sapiens
SLRECELYVQKHNIQALLKDVSIVQLCTARPERPMAFLREYFEKLEKEEAK

SEQ ID NO: 55 - protein, 54 aa, Homo sapiens
SLKGCELYVQLHGIQQVLKDCIVHLCISKPERPMKFLREHFEKLEKEENRQILA

SEQ ID NO: 56 - protein, 44 aa, Homo sapiens
SHIQIPPGLTELLQGYTVEVGQQPPDLVDFAVEYFTRLREARRQ

SEQ ID NO: 57 - protein, 44 aa, Homo sapiens
SIEIPAGLTELLQGFTVEVLRHQPADLLEFALQHFTRLQQENER

SEQ ID NO: 58 - protein, 44 aa, Artificial Sequence
  Description of Artificial Sequence Synthetic polypeptide
XXIXIPPXLXXLLXXYXVXVLXXXPPXLVXFXVXYFXXLXXXXX

SEQ ID NO: 59 - protein, 17 aa, Artificial Sequence
  Description of Artificial Sequence Synthetic peptide
XXXXXAXXIVXXAIXXX

SEQ ID NO: 60 - protein, 17 aa, Artificial Sequence
  Description of Artificial Sequence Synthetic peptide
QIEYVAKQIVDYAIHQA

SEQ ID NO: 61 - protein, 17 aa, Artificial Sequence
  Description of Artificial Sequence Synthetic peptide
QIEYKAKQIVDHAIHQA

SEQ ID NO: 62 - protein, 17 aa, Artificial Sequence
  Description of Artificial Sequence Synthetic peptide
QIEYHAKQIVDHAIHQA

SEQ ID NO: 63 - protein, 17 aa, Artificial Sequence
  Description of Artificial Sequence Synthetic peptide
QIEYVAKQIVDHAIHQA

SEQ ID NO: 64 - protein, 18 aa, Artificial Sequence
  Description of Artificial Sequence Synthetic peptide
PLEYQAGLLVQNAIQQAI

SEQ ID NO: 65 - protein, 18 aa, Artificial Sequence
  Description of Artificial Sequence Synthetic peptide
LLIETASSLVKNAIQLSI

SEQ ID NO: 66 - protein, 18 aa, Artificial Sequence
  Description of Artificial Sequence Synthetic peptide
LIEEAASRIVDAVIEQVK

SEQ ID NO: 67 - protein, 18 aa, Artificial Sequence
  Description of Artificial Sequence Synthetic peptide
ALYQFADRFSELVISEAL

SEQ ID NO: 68 - protein, 17 aa, Artificial Sequence
  Description of Artificial Sequence Synthetic peptide
LEQVANQLADQIIKEAT

SEQ ID NO: 69 - protein, 17 aa, Artificial Sequence
  Description of Artificial Sequence Synthetic peptide
FEELAWKIAKMIWSDVF

SEQ ID NO: 70 - protein, 18 aa, Artificial Sequence
  Description of Artificial Sequence Synthetic peptide
ELVRLSKRLVENAVLKAV

SEQ ID NO: 71 - protein, 18 aa, Artificial Sequence
  Description of Artificial Sequence Synthetic peptide
TAEEVSARIVQVVTAEAV

SEQ ID NO: 72 - protein, 18 aa, Artificial Sequence
  Description of Artificial Sequence Synthetic peptide
QIKQAAFQLISQVILEAT

SEQ ID NO: 73 - protein, 16 aa, Artificial Sequence
  Description of Artificial Sequence Synthetic peptide
LAWKIAKMIVSDVMQQ

SEQ ID NO: 74 - protein, 24 aa, Artificial Sequence
  Description of Artificial Sequence Synthetic peptide
DLIEEAASRIVDAVIEQVKAAGAY

SEQ ID NO: 75 - protein, 18 aa, Artificial Sequence
  Description of Artificial Sequence Synthetic peptide
LEQYANQLADQIIKEATE

SEQ ID NO: 76 - protein, 20 aa, Artificial Sequence
  Description of Artificial Sequence Synthetic peptide
FEELAWKIAKMIWSDVFQQC

SEQ ID NO: 77 - protein, 17 aa, Artificial Sequence
  Description of Artificial Sequence Synthetic peptide
QIEYLAKQIPDNAIQQA

SEQ ID NO: 78 - protein, 25 aa, Artificial Sequence
  Description of Artificial Sequence Synthetic peptide
KGADLIEEAASRIVDAVIEQVKAAG

SEQ ID NO: 79 - protein, 25 aa, Artificial Sequence
  Description of Artificial Sequence Synthetic peptide
KGADLIEEAASRIPDAPIEQVKAAG

SEQ ID NO: 80 - protein, 25 aa, Artificial Sequence
  Description of Artificial Sequence Synthetic peptide
PEDAELVRLSKRLVENAVLKAVQQY

SEQ ID NO: 81 - protein, 25 aa, Artificial Sequence
  Description of Artificial Sequence Synthetic peptide
PEDAELVRTSKRLVENAVLKAVQQY

SEQ ID NO: 82 - protein, 25 aa, Artificial Sequence
  Description of Artificial Sequence Synthetic peptide
PEDAELVRLSKRDVENAVLKAVQQY

SEQ ID NO: 83 - protein, 25 aa, Artificial Sequence
  Description of Artificial Sequence Synthetic peptide
PEDAELVRLSKRLPENAVLKAVQQY

SEQ ID NO: 84 - protein, 25 aa, Artificial Sequence
  Description of Artificial Sequence Synthetic peptide
PEDAELVRLSKRLPENAPLKAVQQY

SEQ ID NO: 85 - protein, 25 aa, Artificial Sequence
  Description of Artificial Sequence Synthetic peptide
PEDAELVRLSKRLVENAVEKAVQQY

SEQ ID NO: 86 - protein, 25 aa, Artificial Sequence
  Description of Artificial Sequence Synthetic peptide
EEGLDRNEEIKRAAFQIISQVISEA

SEQ ID NO: 87 - protein, 25 aa, Artificial Sequence
  Description of Artificial Sequence Synthetic peptide
LVDDPLEYQAGLLVQNAIQQAIAEQ

SEQ ID NO: 88 - protein, 25 aa, Artificial Sequence
  Description of Artificial Sequence Synthetic peptide
QYETLLIETASSLVKNAIQLSIEQL

SEQ ID NO: 89 - protein, 25 aa, Artificial Sequence
  Description of Artificial Sequence Synthetic peptide
LEKQYQEQLEEEVAKVIVSMSIAFA

SEQ ID NO: 90 - protein, 25 aa, Artificial Sequence
  Description of Artificial Sequence Synthetic peptide
NTDEAQEELAWKIAKMIVSDIMQQA

SEQ ID NO: 91 - protein, 25 aa, Artificial Sequence
  Description of Artificial Sequence Synthetic peptide
VNLDKKAVLAEKIVAEAIEKAEREL

SEQ ID NO: 92 - protein, 25 aa, Artificial Sequence
  Description of Artificial Sequence Synthetic peptide
NGILELETKSSKLVQNIIQTAVDQF

SEQ ID NO: 93 - protein, 25 aa, Artificial Sequence
  Description of Artificial Sequence Synthetic peptide
TQDKNYEDELTQVALALVEDVINYA

SEQ ID NO: 94 - protein, 25 aa, Artificial Sequence
  Description of Artificial Sequence Synthetic peptide
ETSAKDNINIEEAARFLVEKILVNH

SEQ ID NO: 95 - protein, 44 aa, Artificial Sequence
  Description of Artificial Sequence Synthetic polypeptide
XHIXIPPGLXELLQGYTXEVLRXQPPDLVEFAXXYFXXLXEXRX

SEQ ID NO: 96 - protein, 21 aa, Artificial Sequence
  Description of Artificial Sequence Synthetic peptide
CGQIEYLAKQIVDNAIQQAGC

SEQ ID NO: 97 - protein, 22 aa, Artificial Sequence
  Description of Artificial Sequence Synthetic peptide
RSQSRSRYYRQRQRSRRRRRRS

SEQ ID NO: 98 - protein, 4 aa, Artificial Sequence
  Description of Artificial Sequence Synthetic peptide
PKSC

SEQ ID NO: 99 - protein, 164 aa, Artificial Sequence
  Description of Artificial Sequence Synthetic polypeptide
QDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSCGGGGSLECGHIQIPPGLTELLQGYTVEVLRQQPPDLVEFAVEYFTRLREARAVEHHHHHH

(19) United States
(12) Patent Application Publication
(10) Pub. No.: US 2013/0078263 A1
(43) Pub. Date: Mar. 28, 2013

(54) Anti-HLA-DR Antibodies Suppress Allogeneic and Xenogeneic Immune Responses to Organ Transplants

(71) Applicant: Immunomedics, Inc., Morris Plains, NJ (US)

(72) Inventors: Tokihiko Sawada, Tokyo (JP); Chien-Hsing Chang, Downingtown, PA (US); David M. Goldenberg, Mendham, NJ (US)

(73) Assignee: IMMUNOMEDICS, INC., Morris Plains, NJ (US)

(21) Appl. No.: 13/623,247

(22) Filed: Sep. 20, 2012

Related U.S. Application Data
(60) Provisional application No. 61/537,906, filed on Sep. 22, 2011.
(60) Provisional application No. 61/595,938, filed on Feb. 7, 2012.

(51) Int. Cl. A61K 39/395; A61P 37/06

(52) U.S. Cl. 424/173.1

(57) ABSTRACT

Disclosed herein are methods and compositions comprising anti-HLA-DR antibodies for treatment of allogeneic and xenogeneic immune responses occurring in organ transplant rejection and other immune dysfunction diseases. In preferred embodiments, the anti-HLA-DR antibodies are effective to deplete antigen-presenting cells, such as dendritic cells. Most preferably, administration of the therapeutic compositions depletes all subsets of APCs, including mDCs, pDCs, B cells and monocytes, without significant depletion of T cells. In alternative embodiments, administration of the therapeutic compositions suppresses proliferation of allo-reactive T cells, while preserving cytomegalovirus (CMV)-specific, CD8⁺ memory T cells. The compositions and methods provide a novel therapeutic agent for suppressing or preventing allogeneic or xenogeneic immune responses, without altering preexisting anti-viral immunity.

### CROSS-REFERENCE TO RELATED APPLICATIONS

This application claims the benefit under 35 U.S.C. 119(e) of provisional application Ser. Nos. 61/537,906, filed Sep. 22, 2011, and 61/595,938, filed Feb. 7, 2012, the entire text of each of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Sep. 18, 2012, is named IMM335US.txt and is 35,864 bytes in size.

### FIELD OF THE INVENTION

The present invention concerns compositions and methods of use of anti-HLA-DR antibodies, antibody fragments, immunoconjugates and complexes thereof (collectively referred to herein as “anti-HLA-DR antibodies”) for treatment of immune dysfunction diseases, including but not limited to organ transplant rejection. Preferably, administration of the therapeutic compositions depletes all subsets of antigen-presenting cells (APCs), including myeloid dendritic cell type 1 (mDC1) and type 2 (mDC2), plasmacytoid dendritic cells (pDCs), B cells and monocytes, without significant depletion of T cells. In more preferred embodiments, administration of anti-HLA-DR antibodies suppresses proliferation of allo-reactive T cells, while preserving cytomegalovirus (CMV)-specific, CD8⁺ memory T cells. Most preferably, the anti-HLA-DR antibodies suppress or prevent allogeneic and/or xenogeneic immune responses after organ transplant, without altering anti-pathogen immunity.

### BACKGROUND

Allogeneic Immune Response

The immunological dogma that CD4+ T cells recognize antigens on major histocompatibility complex (MHC) class II, and that CD8+ T cells recognize antigens on MHC class I, is a central tenet of transplantation immunity (Sherman & Chattopadhyay, Ann Rev Immunol 1993; 11: 385-402; Auchincloss & Sultan, Curr Opin Immunol 1996; 8: 681-687). Recognition of allogeneic antigens by host T cells occurs in two main ways: after presentation by donor MHC on donor antigen-presenting cells (APCs) or endothelial cells of grafts (direct recognition), and after presentation along with self MHC after processing of donor antigens by host APCs (indirect recognition) (Sayech et al., Transplantation 1994; 57: 1295-1302; Shoskes & Wood, Immunol Today 1994; 15: 32-38). In both recognition systems, CD4+ T cells play a major role. CD4+ T cells are activated upon recognition of allogeneic antigens and MHC class II complex, and develop into Th1 or Th2 cells, producing specific cytokines (Mossaman et al., J Immunol 1986; 136: 2348-2367; Arthur & Mason, J Exp Med 1986; 163: 774-786; Abbas et al., Nature 1996). Previous studies have suggested that Th1 cells mediate rejection and that Th2 cells mediate tolerance of grafts (D'Elios et al., Kidney Int 1997; 51: 1876-1884), although this simple paradigm is still in question (Tay et al., Curr Opin Organ Transplant 2009; 14: 16-22; Piccotti et al., Transplantation 1997; 63: 619-624; Bagley et al., Nature Immunol 2000; 1: 257-261).

Blocking of the interaction between CD4⁺ T cells and MHC class II is an attractive strategy, and has been investigated (Kruisbeek et al., J Immunol 1985; 134: 3597-3604). Treatment with anti-MHC class II antibody has been shown to prolong graft survival in allogeneic (Priestley et al., Transplantation 1992; 53: 1024-1032) and concordant xenogeneic (Saxton et al., Transplantation 1999; 67: 1599-1606) transplantation models. However, for human leukocyte antigen (HLA) class II, very few antibodies have been clinically available (Jonker et al., Am J Transplant 2004; 4: 1756-1761) and this has hampered the clinical introduction of such antibodies.

Xenogeneic Immune Response

Xenotransplantation may be the ultimate solution to the current shortage of human organs for transplantation. However, several problems have hampered the clinical application of xenotransplantation, such as hyperacute rejection (HAR), acute humoral xenogeneic rejection (AHXR), and cross-species infection. HAR is a major obstacle, mediated by binding of natural antibodies in human serum to the terminal carbohydrate epitope, Galα(1-3)Galβ(1-4)GlcNAc-R(α-Gal), which is abundantly expressed on xenogeneic organs (Galili, 1995, Immunol Today 16:480-82). HAR has been partially prevented by producing α1-3Gal-knockout xenogeneic donors, including pigs (Chen et al., Nat Med 2005; 11:1295-1298; Hisashi et al., Am J Transplant 2008; 8:2516-2526) and cattle (Sendai et al., Transplantation 2006; 81:760-766). Even if HAR can be prevented, however, AHXR and T cell-mediated cellular rejection remain additional barriers to be overcome. Some studies have indicated that human T-cell responses against xenografts are stronger than those against allografts (Dorling et al., Eur J Immunol 1996; 26:1378-1387). Adequate suppression of T-cell responses is pivotal for achieving successful xenotransplantation.

IMMU-114 is an anti-class II-DR humanized monoclonal antibody designed for use in class II-DR-overexpressing B-cell malignancies (Stein et al., Blood 2006; 108: 2736-2744; Stein et al., Blood 2010; 115: 5180-5190). It was recently reported that IMMU-114 can deplete human antigen-presenting cells leading to suppressed T-cell proliferation in allogeneic MLR (mixed lymphocyte reaction), suggesting therapeutic potential against graft-versus-host disease (Chen et al., Blood 2010; 116: abstract 2544). A need exists for compositions and methods of use of anti-HLA-DR antibodies, such as IMMU-114, to suppress or prevent the allogeneic or xenogeneic immune response that occurs with organ transplantation.

### SUMMARY

The present invention relates to compositions and methods of use of anti-HLA-DR antibodies, such as IMMU-114, that suppress or prevent the allogeneic and xenogeneic immune responses in vitro and in vivo. Many examples of anti-HLA-DR antibodies are known in the art and any such known antibody or fragment thereof may be utilized. In a preferred embodiment, the anti-HLA-DR antibody is an hL243 antibody (also known as IMMU-114) that comprises the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). A humanized L243 anti-HLA-DR antibody suitable for use is disclosed in U.S. Pat. No. 7,612,180, incorporated herein by reference from Col. 46, line 45 through Col. 60, line 50 and FIG. 1 through FIG. 3. However, in alternative embodiments, other known and/or commercially available anti-HLA-DR antibodies may be utilized, such as 1D10 (apolizumab) (Kostelny et al., 2001, Int J Cancer 93:556-65); MS-GPC-1, MS-GPC-6, MS-GPC-8, MS-GPC-10, etc. (U.S. Pat. No. 7,521,047); Lym-1, TAL 8.1, 520B, ML11C11, SPM289, MEM-267, TAL 15.1, TAL 1B5, G-7, 4D12, Bra30 (Santa Cruz Biotechnology, Inc., Santa Cruz, Calif.); TAL 16.1, TU36, C120 (ABCAM®, Cambridge, Mass.); and any other anti-HLA-DR antibody known in the art.

The anti-HLA-DR antibody may be selected such that it competes with or blocks binding to HLA-DR of an L243 antibody comprising the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). Alternatively, the anti-HLA-DR antibody may bind to the same epitope of HLA-DR as an L243 antibody.

The anti-HLA-DR antibodies or fragments thereof may be used as naked antibodies, alone or in combination with one or more therapeutic agents. Alternatively, the antibodies or fragments may be utilized as immunoconjugates, attached to one or more therapeutic agents. (For methods of making immunoconjugates, see, e.g., U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.) Therapeutic agents may be selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide molecule (e.g., an antisense molecule or a gene) or a second antibody or fragment thereof.

The therapeutic agent may be selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin.

The therapeutic agent may comprise a radionuclide selected from the group consisting of ¹⁰³Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{121m}Te, ¹²⁵I, ^{125m}Te, ¹²⁶I, ¹³¹I, ¹³³I, ¹³N, ¹⁴²Pr, ¹⁴³Pr, ¹⁴⁹Pr, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

The therapeutic agent may be an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

An immunomodulator of use may be selected from the group consisting of a cytokine, a stem cell growth factor, a lymphotoxin, a hematopoietic factor, a colony stimulating factor (CSF), an interferon (IFN), erythropoietin, thrombopoietin and combinations thereof. Exemplary immunomodulators may include IL-1, IL-2, IL-3, IL-6, IL-10, IL-12, IL-18, IL-21, interferon-α, interferon-β, interferon-γ, G-CSF, GM-CSF, and mixtures thereof.

Exemplary anti-angiogenic agents may include angiostatin, endostatin, basculostatin, canstatin, maspin, anti-VEGF binding molecules, anti-placental growth factor binding molecules, or anti-vascular growth factor binding molecules.

In certain embodiments, the antibody or fragment may comprise one or more chelating moieties, such as NOTA, DOTA, DTPA, TETA, Tscg-Cys, or Tsca-Cys. In certain embodiments, the chelating moiety may form a complex with a therapeutic or diagnostic cation, such as Group II, Group III, Group IV, Group V, transition, lanthanide or actinide metal cations, Tc, Re, Bi, Cu, As, Ag, Au, At, or Pb.

In certain alternative embodiments, other antibodies that bind to antigens expressed in APCs in general and DCs in particular may be of use, either alone or in combination with anti-HLA-DR antibodies, to suppress or prevent allogeneic and xenogeneic immune responses. A variety of antigens associated with dendritic cells are known in the art, including but not limited to CD209 (DC-SIGN), CD34, CD74, CD205, TLR 2 (toll-like receptor 2), TLR 4, TLR 7, TLR 9, BDCA-2, BDCA-3, BDCA-4, and HLA-DR. In particular alternative embodiments, an antibody of use may be an anti-CD74 antibody. Many examples of antagonistic anti-CD74 antibodies are known in the art and any such known antibody or fragment thereof may be utilized. In a preferred embodiment, the anti-CD74 antibody is an hLL1 antibody (also known as milatuzumab) that comprises the light chain complementarity-determining region (CDR) sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). A humanized LL1 (hLL1) anti-CD74 antibody suitable for use is disclosed in U.S. Pat. No. 7,312,318, incorporated herein by reference from Col. 35, line 1 through Col. 42, line 27 and FIG. 1 through FIG. 1. However, in alternative embodiments, other known and/or commercially available anti-CD74 antibodies may be utilized, such as LS-B1963, LS-B2594, LS-B1859, LS-B2598, LS-05525, LS-C44929, etc. (LSBio, Seattle, Wash.); LN2 (BIOLEGEND®, San Diego, Calif.); PIN.1, SPM523, LN3, CerCLIP.1 (ABCAM®, Cambridge, Mass.); At14/19, Bu45 (SEROTEC®, Raleigh, N.C.); 1D1 (ABNOVA®, Taipei City, Taiwan); 5-329 (EBIOSCIENCE®, San Diego, Calif.); and any other antagonistic anti-CD74 antibody known in the art.

In some embodiments, the antibody or fragment thereof may be a human, chimeric, or humanized antibody or fragment thereof. A humanized antibody or fragment thereof may comprise the complementarity-determining regions (CDRs) of a murine antibody and the constant and framework (FR) region sequences of a human antibody, which may be substituted with at least one amino acid from corresponding FRs of a murine antibody. A chimeric antibody or fragment thereof may include the light and heavy chain variable regions of a murine antibody, attached to human antibody constant regions. The antibody or fragment thereof may include human constant regions of IgG1, IgG2a, IgG3, or IgG4.

In certain preferred embodiments, an anti-HLA-DR antibody complex may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interaction between a dimerization and docking domain (DDD) sequence derived from the regulatory subunit of human cAMP-dependent protein kinase (PKA) and an anchor domain (AD) sequence derived from any of a variety of AKAP proteins. The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences. Although the standard DNL™ complex comprises a trimer with two DDD-linked molecules attached to one AD-linked molecule, variations in complex structure allow the formation of dimers, trimers, tetramers, pentamers, hexamers and other multimers. In some embodiments, the DNL™ complex may comprise two or more antibodies, antibody fragments or fusion proteins which bind to the same antigenic determinant or to two or more different antigens. The DNL™ complex may also comprise one or more other effectors, such as a cytokine or PEG moiety.

Also disclosed is a method for treating and/or diagnosing a disease or disorder that includes administering to a patient a therapeutic and/or diagnostic composition that includes any of the aforementioned antibodies or fragments thereof. Typically, the composition is administered to the patient intravenously, intramuscularly or subcutaneously at a dose of 20-5000 mg. In preferred embodiments, the disease or disorder is an immune dysregulation or autoimmune disease, such as organ-graft rejection or graft-versus-host disease. In more preferred embodiments, the antibody or fragment thereof is effective to suppress or prevent allogeneic or xenogeneic immune responses.

Exemplary autoimmune diseases include acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, or fibrosing alveolitis.

In particularly preferred embodiments, administration of the anti-HLA-DR antibodies or fragments thereof can deplete all subsets of APCs, but not T cells, from human peripheral blood mononuclear cells (PBMCs), including myeloid DCs (mDCs), plasmacytoid DCs (pDCs), B cells, and monocytes. Most preferably, the antibodies or fragments suppress the proliferation of allo-reactive T cells in mixed leukocyte cultures while preserving CMV-specific, CD8⁺ memory T cells.

### BRIEF DESCRIPTION OF THE DRAWINGS

The following Figures are provided to illustrate exemplary, but non-limiting, preferred embodiments of the invention.

FIG. 1. Anti-HLA antibody IMMU-114 depletes all subsets of human PBMCs. Human PBMCs were incubated with 5 μg/ml IMMU-114, control antibodies (hMN-14 and rituximab), or medium only, for 3 days. The effect of each treatment on APC subsets was evaluated by co-staining the cells with PE-labeled anti-CD14 and anti-CD19, in combination with APC-labeled anti-BDCA-1 or anti-BDCA-2, for analysis of mDC1 and pDCs, respectively; or a mixture of FITC-labeled anti-BDCA-2 and APC-labeled anti-BDCA-3 for analysis of mDC2. 7-AAD was added before flow cytometric analyses. PBMCs were gated to exclude debris and dead cells on the basis of their forward and side scatter characteristics. The subpopulations of PBMCs were gated as follows: monocytes, CD14⁺SSC^{medium}; B cells, CD19⁺SSC^{low}; non-B lymphocytes (mostly T cells), CD19⁻CD14⁻SSC^{low}; mDC1, CD14⁻CD19⁻BDCA-1⁺. The live cell fraction of each cell population was determined by measuring 7-AAD^{neg} cells. Mean percentages of live mDC1, mDC2, B cells, monocytes, and non-B lymphocytes in PBMCs, relative to untreated control (Medium), are shown (n=6-7 donors). Error bars, SD; **, P<0.01 vs. hMN-14.

FIG. 2. IMMU-114 is cytotoxic to purified mDC1, mDC2, or pDCs. mDC1, mDC2, and pDCs were isolated from human PBMCs using magnetic beads, and treated for 2 days with IMMU-114 or control antibody hMN-14, followed by 7-AAD staining for flow cytometry analysis of cell viability of mDC1 (FIG. 2A), pDCs (FIG. 2B), and mDC2 (FIG. 2C). The numbers represent the percentages of live cells in the acquired total events. Data shown are representative of 2 donors.

FIG. 3. IMMU-114 reduces T-cell proliferation in allo-MLR cultures. CFSE-labeled PBMCs from two different donors were mixed and incubated with IMMU-114 or control antibody hMN-14 at 5 μg/ml for 11 days, and the cells were harvested and analyzed by flow cytometry. The proliferating cells were quantitated by measuring the CFSE^{low} cell frequencies. The statistical analysis of all combinations of stimulator/responder PBMCs is shown. Error bars, SD, n=10 stimulator/responder combinations from 5 donors. **P<0.01 vs. hMN-14.

FIG. 4 In vitro MLR. Responder cells were co-cultured with self (Self) or allogeneic (Allo) stimulator cells at a responder to stimulator ratio of 1:1, 1:2, 1:4, and 1:8 for 6 days. The cells were cultured in the presence of 10 nM control antibody or IMMU-114. Self+control antibody: clear squares, Self+IMMU-114: solid squares, Allo+control antibody: clear circles, Allo+IMMU-114; solid circles. Statistical analysis was performed by one-way ANOVA.

FIG. 5. CFSE-MLR. Responder cells were co-cultured for 6 days, with self stimulators (Self: A, D), allogeneic stimulators with control antibody (Allo+Control Ab: B, E), or allogeneic stimulators with IMMU-114 (Allo+IMMU-114: C, F). Proliferating CD4+ or CD8+ T cells were visualized by a low intensity of CFSE fluorescence (area surrounded by thick square).

FIG. 6. Phenotypic changes in PBMCs. Phenotypic changes in PBMCs of MLR treated with control antibody or IMMU-114, and resting PBMCs treated with control antibody or IMMU-114 (right column), were analyzed by flow cytometry after 6 days of culture. Representative results are shown. X/Y axes are: (A) CD3/HLA-DR, (B) CD14/HLA-DR, and (C) CD11c/HLA-DR. Frequencies of CD3+ class II-DR+ cells among control antibody- and IMMU-114-treated MLR and resting PBMCs were 52.2% and 4.0%, and 32.5% and 0.5%, respectively (FIG. 6A). Those of CD14+ class II-DR+ cells were 4.8% and 2.2%, and 1.6% and 0.5%, respectively (FIG. 6B), and those among CD11c+ class II-DR+ cells were 15.4% and 2.3%, and 5.5% and 0.4%, respectively (FIG. 6C).

FIG. 7. Concentration of Th1-type cytokines in MLR culture medium. Responder cells were co-cultured with self stimulators (Self: white), allogeneic stimulators with control antibody (Allo+Control Ab: dotted), or allogeneic stimulators with IMMU-114 (Allo+IMMU-114: black). Th1-type cytokines in the culture medium were measured by ELISA. Statistical analysis was performed by one-way ANOVA. SS: statistically significant.

FIG. 8. In vitro MLR. Actual numbers of cell-clusters of Self, Xeno, and IMMU-114 were 2.0±2.1, 36.2±5.0, and 103±13.8, respectively (P=0.0005; one-way ANOVA). SS: statistically significant.

FIG. 9. Thymidine incorporation assay. Responder cells were co-cultured with self (Self) or xenogeneic (Xeno) stimulator cells at a responder to stimulator ratio of 1:1, 1:2, 1:4, and 1:8. The cells were cultured in the presence of 10 nM control antibody or IMMU-114. Self+control antibody (Self: horizontal bar):, Self+IMMU-114 (vertical bar), Xeno+control antibody (white), Xeno+IMMU-114 (white). 1) P=0.036; one-way ANOVA, 2) P=0.092; one-way ANOVA. The figure shows representative results from 5 different experiments.

FIG. 10. CFSE-MLR. Representative results of CFSE-MLR for Self (A, B, C), Xeno (D, E, F), and IMMU-114 (G, H, I) are shown. Frequency of CFSE-low (M1-population) for Xeno (D) is significantly high than in that for Self (A) and IMMU-114 (G) (P=0.023; one-way ANOVA). The frequency of CFSE-low, activating CD4+ T cells for Xeno (B) is significantly higher than that for Self (E) and IMM-114 (H) (P=0.027; one-way ANOVA). The frequency of CFSE-low, CD4-positive, CD25-positive activating T cells for Xeno (C) is significantly higher than that for Self (F) and IMM-114 (I) (P=0.027; one-way ANOVA). The figure shows representative results from 5 different experiments.

FIG. 11. Cytokine concentrations in the culture medium. The concentrations of IL-2 (A), IFN-γ (B), TNF-α (C), IL-6 (D), IL-4 (E), and IL-17 (F) in the in vitro MLR culture medium were measured by ELISA. The concentrations of IL-2 (A) for Self, Xeno and IMMU-114 were 24.4±8.2, 60.2±0.5, and 34.2±2.3 pg/mL, respectively (P=0.007; one-way ANOVA). Those for IFN-γ (B) were 27.5±7.4, 387.9±89.1, and 45.4±18.7 pg/mL, respectively (P=0.005; one-way ANOVA), and those for TNF-α (C) were 27.5±7.4, 387.9±89.1, and 45.4±18.7 pg/mL, respectively (P=0.0008; one-way ANOVA). Those for IL-6 (D) were 17.6±11.1, 52.7±28.8, and 11.4±4.8 pg/mL, respectively (P=0.005; one-way ANOVA), those for IL-4 (E) were 6.0±1.4, 6.8±1.8, and 6.6±1.3 pg/mL, respectively (P=0.4208; one-way ANOVA), and those for IL-17 (F) were 69.8±43.5, 60.9±37.4, and 62.6±28.7 pg/mL, respectively (P=0.8342; one-way ANOVA). SS: statistically significant. The figure shows representative results from 5 different experiments.

FIG. 12. In vivo suppression of xenogeneic immune response by IMMU-114. The xenogeneic immune response was examined in a monkey kidney transplant model. Serum creatinine (Cr) and blood urea nitrogen (BUN) were examined following organ transplant. Addition of IMMU-114 reduced the immune response to organ transplant.

### DETAILED DESCRIPTION

### Definitions

As used herein, the terms “a”, “an” and “the” may refer to either the singular or plural, unless the context otherwise makes clear that only the singular is meant.

An “antibody” refers to a full-length (i.e., naturally occurring or formed by normal immunoglobulin gene fragment recombinatorial processes) immunoglobulin molecule (e.g., an IgG antibody).

An “antibody fragment” is a portion of an antibody such as F(ab′)₂, F(ab)₂, Fab′, Fab, Fv, scFv, single domain antibodies (DABs or VHHs) and the like, including half-molecules of IgG4 (van der Neut Kolfschoten et al. (Science 2007; 317(14 September):1554-1557). Regardless of structure, an antibody fragment binds with the same antigen that is recognized by the intact antibody. For example, an anti-HLA-DR antibody fragment binds with an epitope of HLA-DR. The term “antibody fragment” also includes isolated fragments consisting of the variable regions, such as the “Fv” fragments consisting of the variable regions of the heavy and light chains and recombinant single chain polypeptide molecules in which light and heavy chain variable regions are connected by a peptide linker (“scFv proteins”).

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains. Additional FR amino acid substitutions from the parent, e.g. murine, antibody may be made. The constant domains of the antibody molecule are derived from those of a human antibody.

A “human antibody” is, for example, an antibody obtained from transgenic mice that have been genetically engineered to produce human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

A “therapeutic agent” is an atom, molecule, or compound that is useful in the treatment of a disease. Examples of therapeutic agents include but are not limited to antibodies, antibody fragments, drugs, toxins, enzymes, nucleases, hormones, immunomodulators, antisense oligonucleotides, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

An “immunoconjugate” is a conjugate of an antibody, antibody fragment, antibody fusion protein, bispecific antibody or multispecific antibody with an atom, molecule, or a higher-ordered structure (e.g., with a carrier, a therapeutic agent, or a diagnostic agent). A “naked antibody” is an antibody that is not conjugated to any other agent.

As used herein, the term “antibody fusion protein” is a recombinantly produced antigen-binding molecule in which an antibody or antibody fragment is linked to another protein or peptide, such as the same or different antibody or antibody fragment or a DDD or AD peptide. The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity. For example, a diabody, where one binding site reacts with one antigen and the other with another antigen.

A “bispecific antibody” is an antibody that can bind simultaneously to two targets which are of different structure. Bispecific antibodies (bsAb) and bispecific antibody fragments (bsFab) may have at least one arm that specifically binds to, for example, an APC and/or DC antigen or epitope and at least one other arm that binds to a different antigen or epitope. The second arm may bind to a different APC or DC antigen or it may bind to a targetable conjugate that bears a therapeutic or diagnostic agent. A variety of bispecific antibodies can be produced using molecular engineering.

Xenotransplantation

Xenografts involve transplantation of organs, tissues or cells from one species to a different species. Given the chronic shortage of available organ or tissue transplant donors, xenotransplantation may offer promise to improve and/or prolong the lives of individuals undergoing organ or tissue failure at least temporarily and possibly long-term. Various xenograft techniques have been attempted, with limited success to date. Liver xenografts have been attempted for treatment of acute liver failure (e.g., Hara et al., 2008, Liver Transplant 14:425-34) and it has been suggested that liver from α1,3-galactosyltransferase gene-knockout pigs may survive long enough to function as a bridge to allotransplantation in humans (Id.). Transplant of a pig liver into a human subject has been attempted (Id.). Despite removal of over 90% of the recipient's natural xenoantibodies by plasmapheresis and ex vivo perfusion of the donor pig kidneys before transplantation, the xenoantibodies rapidly returned and resulted in complement-mediated rejection of the graft and death of the recipient within 34 hours.

Later studies of pig to primate xenotransplantation have been reported (e.g., Sachs et al., Transpl Immunol 2009, 21:101-05). The article supports the view that inducing tolerance across xenogeneic barriers is more important for xenotransplantation than use of non-specific immunosuppressive agents (Id.). As of 2009, neither immune tolerance nor successful clinical xenotransplantation had been achieved (Id.), with continued occurrence of hyperacute rejection (HAR), severe delayed xenograft rejection (DXR) and acute cellular rejection (ACR). Only minimal improvement was reported using GalT-KO (knockout) donor kidneys. The rejected GalT-KO kidneys showed severe cellular and humoral rejection (Id.). Greater success was achieved with xenotransplantation in humanized mice (Id.). However, the limited success with GalT-KO (α1,3-galactosyltransferase gene-knockout) donor organs emphasized the importance of nonGal antigens in xenogeneic immune response (Ekser & Cooper, Curr Opin Organ Transplant 2008 13:531-35). Despite progress, a need exists for more effective means of suppressing or preventing immune response in allogeneic and xenogeneic organ transplantation.

Anti-HLA-DR Antibodies

The human leukocyte antigen-DR (HLA-DR) is one of three polymorphic isotypes of the class II major histocompatibility complex (MHC) antigen. Because HLA-DR is expressed at high levels on a range of hematologic malignancies, there has been considerable interest in its development as a target for antibody-based lymphoma therapy. However, safety concerns have been raised regarding the clinical use of HLA-DR-directed antibodies, because the antigen is expressed on normal as well as tumor cells. (Dechant et al., 2003, Semin Oncol 30:465-75) HLA-DR is constitutively expressed on normal B cells, monocytes/macrophages, dendritic cells, and thymic epithelial cells. In addition, interferon-gamma may induce HLA class II expression on other cell types, including activated T and endothelial cells (Dechant et al., 2003).

The most widely recognized function of HLA molecules is the presentation of antigen in the form of short peptides to the antigen receptor of T lymphocytes. In addition, signals delivered via HLA-DR molecules contribute to the functioning of the immune system by up-regulating the activity of adhesion molecules, inducing T-cell antigen counterreceptors, and initiating the synthesis of cytokines. (Nagy and Mooney, 2003, J Mol Med 81:757-65; Scholl et al., 1994, Immunol Today 15:418-22)

As disclosed in the Examples below, humanized anti-HLA-DR antibody, IMMU-114 or hL243γ4P (Stein et al. Blood 108:2736-2744, 2006), can deplete all subsets of APCs, but not T cells, from human peripheral blood mononuclear cells (PBMCs), including myeloid DCs (mDCs), plasmacytoid DCs (pDCs), B cells, and monocytes. In the absence of other human cells or complement, purified mDCs or pDCs were still killed efficiently by IMMU-114, suggesting that IMMU-114 depletes these APCs in PBMCs independently of antibody-dependent cellular cytotoxicity (ADCC) or complement-dependent cytotoxicity (CDC). Furthermore, IMMU-114 suppressed the proliferation of allo-reactive T cells in mixed leukocyte cultures, yet preserved CMV-specific, CD8⁺ memory T cells. Together, these results support the use of IMMU-114 as a novel therapeutic agent for suppressing or preventing allogeneic or xenogeneic immune response, without altering preexisting anti-viral immunity.

Although the Examples below demonstrate the use of IMMU-114 as an exemplary anti-HLA-DR antibody, the skilled artisan will realize that other anti-HLA-DR antibodies known in the art may be utilized in the claimed methods and compositions.

Preparation of Antibodies

The immunoconjugates and compositions described herein may include monoclonal antibodies. Rodent monoclonal antibodies to specific antigens may be obtained by methods known to those skilled in the art. (See, e.g., Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991)).

General techniques for cloning murine immunoglobulin variable domains have been disclosed, for example, by the publication of Orlandi et al., Proc. Nat'l Acad. Sci. USA 86: 3833 (1989). Techniques for constructing chimeric antibodies are well known to those of skill in the art. As an example, Leung et al., Hybridoma 13:469 (1994), disclose how they produced an LL2 chimera by combining DNA sequences encoding the Vₖ and V_{H} domains of LL2 monoclonal antibody, an anti-CD22 antibody, with respective human and IgG1 constant region domains. This publication also provides the nucleotide sequences of the LL2 light and heavy chain variable regions, Vₖ and V_{H}, respectively. Techniques for producing humanized antibodies are disclosed, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993).

A chimeric antibody is a recombinant protein that contains the variable domains including the CDRs derived from one species of animal, such as a rodent antibody, while the remainder of the antibody molecule; i.e., the constant domains, is derived from a human antibody. Accordingly, a chimeric monoclonal antibody can also be humanized by replacing the sequences of the murine FR in the variable domains of the chimeric antibody with one or more different human FR. Specifically, mouse CDRs are transferred from heavy and light variable chains of the mouse immunoglobulin into the corresponding variable domains of a human antibody. As simply transferring mouse CDRs into human FRs often results in a reduction or even loss of antibody affinity, additional modification might be required in order to restore the original affinity of the murine antibody. This can be accomplished by the replacement of one or more some human residues in the FR regions with their murine counterparts to obtain an antibody that possesses good binding affinity to its epitope. (See, e.g., Tempest et al., Biotechnology 9:266 (1991) and Verhoeyen et al., Science 239: 1534 (1988)).

A fully human antibody can be obtained from a transgenic non-human animal. (See, e.g., Mendez et al., Nature Genetics, 15: 146-156, 1997; U.S. Pat. No. 5,633,425.) Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as an immune dysfunction disease (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.) Recombinant Fab were cloned from the μ, γ and κ chain antibody repertoires and inserted into a phage display library (Id.) RNAs were converted to cDNAs and used to make Fab cDNA libraries using specific primers against the heavy and light chain immunoglobulin sequences (Marks et al., 1991, J. Mol. Biol. 222:581-97). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XENOMOUSE® (e.g., Green et al., 1999, J. Immunol. Methods 231:11-23, incorporated herein by reference) from Abgenix (Fremont, Calif.). In the XENOMOUSE® and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Ig kappa loci, including the majority of the variable region sequences, along accessory genes and regulatory sequences. The human variable region repertoire may be used to generate antibody producing B cells, which may be processed into hybridomas by known techniques. A XENOMOUSE® immunized with a target antigen will produce human antibodies by the normal immune response, which may be harvested and/or produced by standard techniques discussed above. A variety of strains of XENOMOUSE® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XENOMOUSE® system but may utilize any transgenic animal that has been genetically engineered to produce human antibodies.

Known Antibodies

In various embodiments, the claimed methods and compositions may utilize any of a variety of antibodies known in the art. Antibodies of use may be commercially obtained from a number of known sources. For example, a variety of antibody secreting hybridoma lines are available from the American Type Culture Collection (ATCC, Manassas, Va.). A large number of antibodies against various disease targets have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040; 6,451,310; 6,444,206; 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art.

Exemplary known antibodies include, but are not limited to, hPAM4 (U.S. Pat. No. 7,282,567), hA20 (U.S. Pat. No. 7,251,164), hA19 (U.S. Pat. No. 7,109,304), hIMMU31 (U.S. Pat. No. 7,300,655), hLL1 (U.S. Pat. No. 7,312,318), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. Provisional Patent Application 61/145,896), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575). Other known antibodies are disclosed, for example, in U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20040202666 (now abandoned); 20050271671; and 20060193865. The text of each recited patent or application is incorporated herein by reference with respect to the Figures and Examples sections.

Antibody Fragments

Antibody fragments which recognize specific epitopes can be generated by known techniques. The antibody fragments are antigen binding portions of an antibody, such as F(ab)₂, Fab′, Fab, Fv, scFv and the like. Other antibody fragments include, but are not limited to, F(ab′)₂ fragments which can be produced by pepsin digestion of the antibody molecule and Fab′ fragments which can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are disclosed in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991).

An antibody fragment can be prepared by known methods, for example, as disclosed by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

A single complementarity-determining region (CDR) is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. (See, e.g., Larrick et al., Methods: A Companion to Methods in Enzymology 2: 106 (1991); Courtenay-Luck, “Genetic Manipulation of Monoclonal Antibodies,” in MONOCLONAL ANTIBODIES: PRODUCTION, ENGINEERING AND CLINICAL APPLICATION, Ritter et al. (eds.), pages 166-179 (Cambridge University Press 1995); and Ward et al., “Genetic Manipulation and Expression of Antibodies,” in MONOCLONAL ANTIBODIES: PRINCIPLES AND APPLICATIONS, Birch et al., (eds.), pages 137-185 (Wiley-Liss, Inc. 1995).

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 2006, 43:1912-19; Tanha et al., J. Biol. Chem. 2001, 276:24774-780).

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797).

Multispecific and Multivalent Antibodies

Various embodiments may concern use of multispecific and/or multivalent antibodies. For example, an anti-HLA-DR antibody or fragment thereof may be joined together with another protein, peptide, antibody, antibody fragment or other therapeutic or diagnostic agent by any means known in the art. For example, the anti-HLA-DR antibody could be combined with another antibody against a different epitope of the same antigen, or alternatively with an antibody against another antigen expressed by the APC or DC cell, such as CD209 (DC-SIGN), CD34, CD74, CD205, TLR 2 (toll-like receptor 2), TLR 4, TLR 7, TLR 9, BDCA-2, BDCA-3, BDCA-4 or HLA-DR.

Methods for producing bispecific antibodies include engineered recombinant antibodies which have additional cysteine residues so that they crosslink more strongly than the more common immunoglobulin isotypes. (See, e.g., FitzGerald et al, Protein Eng 10:1221-1225, 1997). Another approach is to engineer recombinant fusion proteins linking two or more different single-chain antibody or antibody fragment segments with the needed dual specificities. (See, e.g., Coloma et al., Nature Biotech. 15:159-163, 1997). A variety of bispecific antibodies can be produced using molecular engineering. In one form, the bispecific antibody may consist of, for example, a scFv with a single binding site for one antigen and a Fab fragment with a single binding site for a second antigen. In another form, the bispecific antibody may consist of, for example, an IgG with two binding sites for one antigen and two scFv with two binding sites for a second antigen.

Diabodies, Triabodies and Tetrabodies

The compositions disclosed herein may also include functional bispecific single-chain antibodies (bscAb), also called diabodies. (See, e.g., Mack et al., Proc. Natl. Acad. Sci., 92: 7021-7025, 1995). For example, bscAb are produced by joining two single-chain Fv fragments via a glycine-serine linker using recombinant methods. The V light-chain (V_{L}) and V heavy-chain (V_{H}) domains of two antibodies of interest are isolated using standard PCR methods. The V_{L} and V_{H} cDNAs obtained from each hybridoma are then joined to form a single-chain fragment in a two-step fusion PCR. The first PCR step introduces the linker, and the second step joins the V_{L} and V_{H} amplicons. Each single chain molecule is then cloned into a bacterial expression vector. Following amplification, one of the single-chain molecules is excised and sub-cloned into the other vector, containing the second single-chain molecule of interest. The resulting bscAb fragment is subcloned into a eukaryotic expression vector. Functional protein expression can be obtained by transfecting the vector into Chinese Hamster Ovary cells.

For example, a humanized, chimeric or human anti-HLA-DR monoclonal antibody can be used to produce antigen specific diabodies, triabodies, and tetrabodies. The monospecific diabodies, triabodies, and tetrabodies bind selectively to targeted antigens and as the number of binding sites on the molecule increases, the affinity for the target cell increases and a longer residence time is observed at the desired location. For diabodies, the two chains comprising the V_{H} polypeptide of the humanized HLA-DR antibody connected to the V_{K} polypeptide of the humanized HLA-DR antibody by a five amino acid residue linker may be utilized. Each chain forms one half of the diabody. In the case of triabodies, the three chains comprising V_{H} polypeptide of the humanized HLA-DR antibody connected to the V_{K} polypeptide of the humanized HLA-DR antibody by no linker may be utilized. Each chain forms one third of the triabody.

More recently, a tetravalent tandem diabody (termed tandab) with dual specificity has also been reported (Cochlovius et al., Cancer Research (2000) 60: 4336-4341). The bispecific tandab is a dimer of two identical polypeptides, each containing four variable domains of two different antibodies (V_{H1}, V_{L1}, V_{H2}, V_{L2}) linked in an orientation to facilitate the formation of two potential binding sites for each of the two different specificities upon self-association.

Dock-and-Lock™

In certain preferred embodiments, bispecific or multispecific antibodies may be produced as a DNL™ complex (see, e.g., U.S. Pat. Nos. 7,521,056; 7,550,143; 7,534,866; 7,527,787 and 7,666,400; the Examples section of each of which is incorporated herein by reference). The method exploits specific protein/protein interactions that occur between the regulatory (R) subunits of cAMP-dependent protein kinase (PKA) and the anchoring domain (AD) of A-kinase anchoring proteins (AKAPs) (Baillie et al., FEBS Letters. 2005; 579: 3264. Wong and Scott, Nat. Rev. Mol. Cell Biol. 2004; 5: 959). PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RID, and each type has α and β isoforms (Scott, Pharmacol. Ther. 1991; 50:123). Thus, there are four types of PKA regulatory subunits—RIα, RIβ, RIIα and RIIβ. The R subunits have been isolated only as stable dimers and the dimerization domain has been shown to consist of the first 44 amino-terminal residues (Newlon et al., Nat. Struct. Biol. 1999; 6:222). Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265; 21561).

Since the first AKAP, microtubule-associated protein-2, was characterized in 1984 (Lohmann et al., Proc. Natl. Acad. Sci USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIα are both located within the same N-terminal 44 amino acid sequence (Newlon et al., Nat. Struct. Biol. 1999; 6:222; Newlon et al., EMBO J. 2001; 20:1651), which is termed the DDD herein.

We have developed a platform technology to utilize the DDD of human PKA regulatory subunit and the AD of AKAP as an excellent pair of linker modules for docking any two entities, referred to hereafter as A and B, into a noncovalent complex, which could be further locked into a stably tethered structure through the introduction of cysteine residues into both the DDD and AD at strategic positions to facilitate the formation of disulfide bonds. The general methodology is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically. Using various combinations of linkers, adaptor modules and precursors, a wide variety of DNL™ constructs of different stoichiometry may be produced and used, including but not limited to dimeric, trimeric, tetrameric, pentameric and hexameric DNL™ constructs (see, e.g., U.S. Pat. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. Utilizing the fusion protein method of constructing AD and DDD conjugated effectors described in the Examples below, virtually any protein or peptide may be incorporated into a DNL™ construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

The skilled artisan will realize that the technique may be utilized to produce complexes comprising multiple copies of the same anti-HLA-DR antibody, or to attach an anti-HLA-DR antibody to an antibody that binds to a different antigen expressed by APCs and/or DCs. Alternatively, the technique may be used to attach antibodies to different effector moieties, such as toxins, cytokines, carrier proteins for siRNA and other known effectors.

Amino Acid Substitutions

In various embodiments, the disclosed methods and compositions may involve production and use of proteins or peptides with one or more substituted amino acid residues. For example, the DDD and/or AD sequences used to make DNL™ constructs may be modified as discussed below.

The skilled artisan will be aware that, in general, amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL website at rockefeller.edu) For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

Pre-Targeting

In certain alternative embodiments, therapeutic agents may be administered by a pretargeting method, utilizing bispecific or multispecific antibodies. In pretargeting, the bispecific or multispecific antibody comprises at least one binding arm that binds to an antigen exhibited by a targeted cell or tissue, such as HLA-DR, while at least one other binding arm binds to a hapten on a targetable construct. The targetable construct comprises one or more haptens and one or more therapeutic and/or diagnostic agents.

Pre-targeting is a multistep process originally developed to resolve the slow blood clearance of directly targeting antibodies, which contributes to undesirable toxicity to normal tissues such as bone marrow. With pre-targeting, a radionuclide or other diagnostic or therapeutic agent is attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

Immunoconjugates

In preferred embodiments, an antibody or antibody fragment may be directly attached to one or more therapeutic agents to form an immunoconjugate. Therapeutic agents may be attached, for example to reduced SH groups and/or to carbohydrate side chains. A therapeutic agent can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the therapeutic agent can be conjugated via a carbohydrate moiety in the Fc region of the antibody.

Methods for conjugating functional groups to antibodies via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, the Examples section of which is incorporated herein by reference. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); U.S. Pat. Nos. 5,443,953 and 6,254,868, the Examples section of which is incorporated herein by reference. The engineered carbohydrate moiety is used to attach the therapeutic or diagnostic agent.

An alternative method for attaching therapeutic agents to a targeting molecule involves use of click chemistry reactions. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. Although the copper catalyst is toxic to living cells, the copper-based click chemistry reaction may be used in vitro for immunoconjugate formation.

A copper-free click reaction has been proposed for covalent modification of biomolecules. (See, e.g., Agard et al., 2004, J Am Chem Soc 126:15046-47.) The copper-free reaction uses ring strain in place of the copper catalyst to promote a [3+2] azide-alkyne cycloaddition reaction (Id.) For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions (Id.)

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.) Examples of such substituted cyclooctynes include difluorinated cyclooctynes, 4-dibenzocyclooctynol and azacyclooctyne (Id.) An alternative copper-free reaction involved strain-promoted akyne-nitrone cycloaddition to give N-alkylated isoxazolines (Id.) The reaction was reported to have exceptionally fast reaction kinetics and was used in a one-pot three-step protocol for site-specific modification of peptides and proteins (Id.) Nitrones were prepared by the condensation of appropriate aldehydes with N-methylhydroxylamine and the cycloaddition reaction took place in a mixture of acetonitrile and water (Id.) These and other known click chemistry reactions may be used to attach therapeutic agents to antibodies in vitro.

The specificity of the click chemistry reaction may be used as a substitute for the antibody-hapten binding interaction used in pretargeting with bispecific antibodies. In this alternative embodiment, the specific reactivity of e.g., cyclooctyne moieties for azide moieties or alkyne moieties for nitrone moieties may be used in an in vivo cycloaddition reaction. An antibody or other targeting molecule is activated by incorporation of a substituted cyclooctyne, an azide or a nitrone moiety. A targetable construct is labeled with one or more diagnostic or therapeutic agents and a complementary reactive moiety. I.e., where the targeting molecule comprises a cyclooctyne, the targetable construct will comprise an azide; where the targeting molecule comprises a nitrone, the targetable construct will comprise an alkyne, etc. The activated targeting molecule is administered to a subject and allowed to localize to a targeted cell, tissue or pathogen, as disclosed for pretargeting protocols. The reactive labeled targetable construct is then administered. Because the cyclooctyne, nitrone or azide on the targetable construct is unreactive with endogenous biomolecules and highly reactive with the complementary moiety on the targeting molecule, the specificity of the binding interaction results in the highly specific binding of the targetable construct to the tissue-localized targeting molecule.

Therapeutic Agents

A wide variety of therapeutic reagents can be administered concurrently or sequentially with the anti-HLA-DR antibodies. For example, drugs, toxins, oligonucleotides, immunomodulators, hormones, hormone antagonists, enzymes, enzyme inhibitors, radionuclides, angiogenesis inhibitors, other antibodies or fragments thereof, etc. The therapeutic agents recited here are those agents that also are useful for administration separately with an antibody or fragment thereof as described above. Therapeutic agents include, for example, cytotoxic agents such as vinca alkaloids, anthracyclines, gemcitabine, epipodophyllotoxins, taxanes, antimetabolites, alkylating agents, antibiotics, SN-38, COX-2 inhibitors, antimitotics, anti-angiogenic and pro-apoptotic agents, particularly doxorubicin, methotrexate, taxol, CPT-11, camptothecans, proteosome inhibitors, mTOR inhibitors, HDAC inhibitors, tyrosine kinase inhibitors, and others.

Other useful cytotoxic agents include nitrogen mustards, alkyl sulfonates, nitrosoureas, triazenes, folic acid analogs, COX-2 inhibitors, antimetabolites, pyrimidine analogs, purine analogs, platinum coordination complexes, mTOR inhibitors, tyrosine kinase inhibitors, proteosome inhibitors, HDAC inhibitors, camptothecins, hormones, and the like. Suitable cytotoxic agents are described in REMINGTON'S PHARMACEUTICAL SCIENCES, 19th Ed. (Mack Publishing Co. 1995), and in GOODMAN AND GILMAN'S THE PHARMACOLOGICAL BASIS OF THERAPEUTICS, 7th Ed. (MacMillan Publishing Co. 1985), as well as revised editions of these publications.

In a preferred embodiment, conjugates of camptothecins and related compounds, such as SN-38, may be conjugated to an anti-HLA-DR antibody, for example as disclosed in U.S. Pat. No. 7,591,994, the Examples section of which is incorporated herein by reference.

A toxin can be of animal, plant or microbial origin. A toxin, such as Pseudomonas exotoxin, may also be complexed to or form the therapeutic agent portion of an immunoconjugate. Other toxins include ricin, abrin, ribonuclease (RNase), DNase I, Staphylococcal enterotoxin-A, pokeweed antiviral protein, onconase, gelonin, diphtheria toxin, Pseudomonas exotoxin, and Pseudomonas endotoxin. See, for example, Pastan et al., Cell 47:641 (1986), Goldenberg, C A—A Cancer Journal for Clinicians 44:43 (1994), Sharkey and Goldenberg, C A—A Cancer Journal for Clinicians 56:226 (2006). Additional toxins suitable for use are known to those of skill in the art and are disclosed in U.S. Pat. No. 6,077,499, the Examples section of which is incorporated herein by reference.

As used herein, the term “immunomodulator” includes cytokines, lymphokines, monokines, stem cell growth factors, lymphotoxins, hematopoietic factors, colony stimulating factors (CSF), interferons (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, transforming growth factor (TGF), TGF-α, TGF-β, insulin-like growth factor (IGF), erythropoietin, thrombopoietin, tumor necrosis factor (TNF), TNF-α, TNF-β, mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-α, interferon-β, interferon-γ, S1 factor, IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21, IL-25, LIF, kit-ligand, FLT-3, angiostatin, thrombospondin, endostatin, LT, and the like.

The antibody or fragment thereof may be administered as an immunoconjugate comprising one or more radioactive isotopes useful for treating diseased tissue. Particularly useful therapeutic radionuclides include, but are not limited to ¹¹¹In, ¹⁷¹Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. The therapeutic radionuclide preferably has a decay energy in the range of 20 to 6,000 keV, preferably in the ranges 60 to 200 keV for an Auger emitter, 100-2,500 keV for a beta emitter and 4,000-6,000 keV for an alpha emitter. Maximum decay energies of useful beta-particle-emitting nuclides are preferably 20-5,000 keV, more preferably 100-4,000 keV and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, I-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213 and Fm-255. Decay energies of useful alpha-particle-emitting radionuclides are preferably 2,000-10,000 keV, more preferably 3,000-8,000 keV, and most preferably 4,000-7,000 keV.

Additional potential therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

Interference RNA

In certain preferred embodiments the therapeutic agent may be a siRNA or interference RNA species. The siRNA, interference RNA or therapeutic gene may be attached to a carrier moiety that is conjugated to an antibody or fragment thereof. A variety of carrier moieties for siRNA have been reported and any such known carrier may be incorporated into a therapeutic antibody for use. Non-limiting examples of carriers include protamine (Rossi, 2005, Nat Biotech 23:682-84; Song et al., 2005, Nat Biotech 23:709-17); dendrimers such as PAMAM dendrimers (Pan et al., 2007, Cancer Res. 67:8156-8163); polyethylenimine (Schiffelers et al., 2004, Nucl Acids Res 32:e149); polypropyleneimine (Taratula et al., 2009, J Control Release 140:284-93); polylysine (Inoue et al., 2008, J Control Release 126:59-66); histidine-containing reducible polycations (Stevenson et al., 2008, J Control Release 130:46-56); histone H1 protein (Haberland et al., 2009, Mol Biol Rep 26:1083-93); cationic comb-type copolymers (Sato et al., 2007, J Control Release 122:209-16); polymeric micelles (U.S. Patent Application Publ. No. 20100121043); and chitosan-thiamine pyrophosphate (Rojanarata et al., 2008, Pharm Res 25:2807-14). The skilled artisan will realize that in general, polycationic proteins or polymers are of use as siRNA carriers. The skilled artisan will further realize that siRNA carriers can also be used to carry other oligonucleotide or nucleic acid species, such as anti-sense oligonucleotides or short DNA genes.

Known siRNA species of potential use include those specific for IKK-gamma (U.S. Pat. No. 7,022,828); VEGF, Flt-1 and Flk-1/KDR (U.S. Pat. No. 7,148,342); Bcl2 and EGFR (U.S. Pat. No. 7,541,453); CDC20 (U.S. Pat. No. 7,550,572); transducin (beta)-like 3 (U.S. Pat. No. 7,576,196); K-ras (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Mirus Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL™ complexes.

Exemplary siRNA species known in the art are listed in Table 1. Although siRNA is delivered as a double-stranded molecule, for simplicity only the sense strand sequences are shown in Table 1.

TABLE 1
Exemplary siRNA Sequences

| Target | Sequence | SEQ ID NO |
|---|---|---|
| VEGF R2 | AATGCGGCGGTGGTGACAGTA | SEQ ID NO: 13 |
| VEGF R2 | AAGCTCAGCACACAGAAAGAC | SEQ ID NO: 14 |
| CXCR4 | UAAAAUCUUCCUGCCCACCdTdT | SEQ ID NO: 15 |
| CXCR4 | GGAAGCUGUUGGCUGAAAAdTdT | SEQ ID NO: 16 |
| PPARC1 | AAGACCAGCCUCUUUGCCCAG | SEQ ID NO: 17 |
| Dynamin 2 | GGACCAGGCAGAAAACGAG | SEQ ID NO: 18 |
| Catenin | CUAUCAGGAUGACGCGG | SEQ ID NO: 19 |
| E1A binding | UGACACAGGCAGGCUUGACUU | SEQ ID NO: 20 |
| protein | | |
| Plasminogen | GGTGAAGAAGGGCGTCCAA | SEQ ID NO: 21 |
| activator | | |
| K-ras | GATCCGTTGGAGCTGTTGGCGTAGTT | SEQ ID NO: 22 |
| | CAAGAGACTCGCCAACAGCTCCAACT | |
| | TTTGGAAA | |
| Sortilin 1 | AGGTGGTGTTAACAGCAGAG | SEQ ID NO: 23 |
| Apolipoprotein E | AAGGTGGAGCAAGCGGTGGAG | SEQ ID NO: 24 |
| Apolipoprotein E | AAGGAGTTGAAGGCCGACAAA | SEQ ID NO: 25 |
| Bcl-X | UAUGGAGCUGCAGAGGAUGdTdT | SEQ ID NO: 26 |
| Raf-1 | TTTGAATATCTGTGCTGAGAACACA | SEQ ID NO: 27 |
| | GTTCTCAGCACAGATATTCTTTTT | |
| Heat shock | AATGAGAAAAGCAAAAGGTGCCCTGTCTC | SEQ ID NO: 28 |
| transcription | | |
| factor 2 | | |
| IGFBP3 | AAUCAUCAUCAAGAAAGGGCA | SEQ ID NO: 29 |
| Thioredoxin | AUGACUGUCAGGAUGUUGCdTdT | SEQ ID NO: 30 |
| CD44 | GAACGAAUCCUGAAGACAUCU | SEQ ID NO: 31 |
| MMP14 | AAGCCTGGCTACAGCAATATGCCTGTCTC | SEQ ID NO: 32 |
| MAPKAPK2 | UGACCAUCACCGAGUUUAUdTdT | SEQ ID NO: 33 |
| FGFR1 | AAGTCGGACGCAACAGAGAAA | SEQ ID NO: 34 |
| ERBB2 | CUACCUUUCUACGGACGUGdTdT | SEQ ID NO: 35 |
| BCL2L1 | CTGCCTAAGGCGGATTTGAAT | SEQ ID NO: 36 |
| ABL1 | TTAUUCCUUCUUCGGGAAGUC | SEQ ID NO: 37 |
| CEACAM1 | AACCTTCTGGAACCCGCCCAC | SEQ ID NO: 38 |
| CD9 | GAGCATCTTCGAGCAAGAA | SEQ ID NO: 39 |
| CD151 | CATGTGGCACCGTTTGCCT | SEQ ID NO: 40 |
| Caspase 8 | AACTACCAGAAAGGTATACCT | SEQ ID NO: 41 |
| BRCA1 | UCACAGUGUCCUUUAUGUAdTdT | SEQ ID NO: 42 |
| p53 | GCAUGAACCGGAGGCCCAUTT | SEQ ID NO: 43 |
| CEACAM6 | CCGGACAGTTCCATGTATA | SEQ ID NO: 44 |

The skilled artisan will realize that Table 1 represents a very small sampling of the total number of siRNA species known in the art, and that any such known siRNA may be utilized in the claimed methods and compositions.

Methods of Therapeutic Treatment

The claimed methods and compositions are of use for treating disease states, such as the allogeneic or xenogeneic immune response from organ transplant. The methods may comprise administering a therapeutically effective amount of a therapeutic antibody or fragment thereof or an immunoconjugate, either alone or in conjunction with one or more other therapeutic agents, administered either concurrently or sequentially.

Multimodal therapies may include therapy with other antibodies, such as anti-CD209 (DC-SIGN), anti-CD34, anti-CD74, anti-CD205, anti-TLR-2, anti-TLR-4, anti-TLR-7, anti-TLR-9, anti-BDCA-2, anti-BDCA-3, anti-BDCA-4 or anti-HLA-DR (including the invariant chain) antibodies in the form of naked antibodies, fusion proteins, or as immunoconjugates. Various antibodies of use are known to those of skill in the art. See, for example, Ghetie et al., Cancer Res. 48:2610 (1988); Hekman et al., Cancer Immunol. Immunother. 32:364 (1991); Longo, Curr. Opin. Oncol. 8:353 (1996), U.S. Pat. Nos. 5,798,554; 6,187,287; 6,306,393; 6,676,924; 7,109,304; 7,151,164; 7,230,084; 7,230,085; 7,238,785; 7,238,786; 7,282,567; 7,300,655; 7,312,318; 7,612,180; 7,501,498; the Examples section of each of which is incorporated herein by reference.

In another form of multimodal therapy, subjects receive therapeutic antibodies in conjunction with standard chemotherapy. For example, cyclophosphamide, etoposide, carmustine, vincristine, procarbazine, prednisone, doxorubicin, methotrexate, bleomycin, dexamethasone or leucovorin, alone or in combination. Additional useful drugs include phenyl butyrate, bendamustine, and bryostatin-1. In a preferred multimodal therapy, both cytotoxic drugs and cytokines are co-administered with a therapeutic antibody. The cytokines, cytotoxic drugs and therapeutic antibody can be administered in any order, or together.

Therapeutic antibodies or fragments thereof can be formulated according to known methods to prepare pharmaceutically useful compositions, whereby the therapeutic antibody is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The therapeutic antibody can be formulated for intravenous administration via, for example, bolus injection or continuous infusion. Preferably, the therapeutic antibody is infused over a period of less than about 4 hours, and more preferably, over a period of less than about 3 hours. For example, the first 25-50 mg could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The therapeutic antibody may also be administered to a mammal subcutaneously or even by other parenteral routes. Moreover, the administration may be by continuous infusion or by single or multiple boluses. Preferably, the therapeutic antibody is infused over a period of less than about 4 hours, and more preferably, over a period of less than about 3 hours.

More generally, the dosage of an administered therapeutic antibody for humans will vary depending upon such factors as the patient's age, weight, height, sex, general medical condition and previous medical history. It may be desirable to provide the recipient with a dosage of therapeutic antibody that is in the range of from about 1 mg/kg to 25 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy.

Alternatively, a therapeutic antibody may be administered as one dosage every 2 or 3 weeks, repeated for a total of at least 3 dosages. Or, the therapeutic antibody may be administered twice per week for 4-6 weeks. If the dosage is lowered to approximately 200-300 mg/m² (340 mg per dosage for a 1.7-m patient, or 4.9 mg/kg for a 70 kg patient), it may be administered once or even twice weekly for 4 to 10 weeks. Alternatively, the dosage schedule may be decreased, namely every 2 or 3 weeks for 2-3 months. It has been determined, however, that even higher doses, such as 20 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

Additional pharmaceutical methods may be employed to control the duration of action of the therapeutic immunoconjugate or naked antibody. Control release preparations can be prepared through the use of polymers to complex or adsorb the immunoconjugate or naked antibody. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). The rate of release of an immunoconjugate or antibody from such a matrix depends upon the molecular weight of the immunoconjugate or antibody, the amount of immunoconjugate or antibody within the matrix, and the size of dispersed particles. Saltzman et al., Biophys. J. 55: 163 (1989); Sherwood et al., supra. Other solid dosage forms are described in Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

Therapy of Autoimmune Disease

Anti-HLA-DR antibodies or immunoconjugates can be used to treat immune dysregulation disease and related autoimmune diseases. Immune diseases may include acute idiopathic thrombocytopenic purpura, Addison's disease, adult respiratory distress syndrome (ARDS), agranulocytosis, allergic conditions, allergic encephalomyelitis, allergic neuritis, amyotrophic lateral sclerosis (ALS), ankylosing spondylitis, antigen-antibody complex mediated diseases, anti-glomerular basement membrane disease, anti-phospholipid antibody syndrome, aplastic anemia, arthritis, asthma, atherosclerosis, autoimmune disease of the testis and ovary, autoimmune endocrine diseases, autoimmune myocarditis, autoimmune neutropenia, autoimmune polyendocrinopathies, autoimmune polyglandular syndromes (or polyglandular endocrinopathy syndromes), autoimmune thrombocytopenia, Bechet disease, Berger's disease (IgA nephropathy), bronchiolitis obliterans (non-transplant), bullous pemphigoid, Castleman's syndrome, Celiac sprue (gluten enteropathy), central nervous system (CNS) inflammatory disorders, chronic active hepatitis, chronic idiopathic thrombocytopenic purpura dermatomyositis, colitis, conditions involving infiltration of T cells and chronic inflammatory responses, coronary artery disease, Crohn's disease, cryoglobulinemia, dermatitis, dermatomyositis, diabetes mellitus, diseases involving leukocyte diapedesis, eczema, encephalitis, erythema multiforme, erythema nodosum, Factor VIII deficiency, fibrosing alveolitis, giant cell arteritis, glomerulonephritis, Goodpasture's syndrome, graft versus host disease (GVHD), granulomatosis, Grave's disease, Guillain-Barre Syndrome, Hashimoto's thyroiditis, hemophilia A, Henoch-Schonlein purpura, idiopathic hypothyroidism, idiopathic thrombocytopenic purpura (ITP), IgA nephropathy, IgA nephropathy, IgM mediated neuropathy, immune complex nephritis, immune hemolytic anemia including autoimmune hemolytic anemia (AIHA), immune responses associated with acute and delayed hypersensitivity mediated by cytokines and T-lymphocytes, immune-mediated thrombocytopenias, juvenile onset diabetes, juvenile rheumatoid arthritis, Lambert-Eaton Myasthenic Syndrome, large vessel vasculitis, leukocyte adhesion deficiency, leukopenia, lupus nephritis, lymphoid interstitial pneumonitis (HIV), medium vessel vasculitis, membranous nephropathy, meningitis, multiple organ injury syndrome, multiple sclerosis, myasthenia gravis, osteoarthritis, pancytopenia, pemphigoid bullous, pemphigus vulgaris, pernicious anemia, polyarteritis nodosa, polychondritis, polyglandular syndromes, polymyalgia, polymyositis, post-streptococcal nephritis, primary biliary cirrhosis, primary hypothyroidism, psoriasis, psoriatic arthritis, pure red cell aplasia (PRCA), rapidly progressive glomerulonephritis, Reiter's disease, respiratory distress syndrome, responses associated with inflammatory bowel disease, Reynaud's syndrome, rheumatic fever, rheumatoid arthritis, sarcoidosis, scleroderma, Sjogren's syndrome, solid organ transplant rejection, Stevens-Johnson syndrome, stiff-man syndrome, subacute thyroiditis, Sydenham's chorea, systemic lupus erythematosus (SLE), systemic scleroderma and sclerosis, tabes dorsalis, Takayasu's arteritis, thromboangitis obliterans, thrombotic thrombocytopenic purpura (TTP), thyrotoxicosis, toxic epidermal necrolysis, tuberculosis, Type I diabetes, ulcerative colitis, uveitis, vasculitis (including ANCA) and Wegener's granulomatosis. In a particularly preferred embodiment, the disease to be treated is organ transplant rejection.

### EXAMPLES

Various embodiments of the present invention are illustrated by the following examples, without limiting the scope thereof.

### Example 1

### Depletion of all Antigen-Presenting Cells by Humanized Anti-HLA-Dr Antibody

IMMU-114 is a humanized IgG4 anti-HLA-DR antibody derived from the murine anti-human HLA-DR antibody, L243. It recognizes a conformational epitope in the α-chain of HLA-DR (Stein et al., 2006, Blood 108:2736-2744). The engineered IgG4 isotype (hL243γ4P) of this humanized antibody abrogates its ADCC and CDC effector functions, but retains its antigen-binding properties and direct cytotoxicity against a variety of tumors (Stein et al., 2006, Blood 108:2736-2744), which is mediated through hyper-activation of ERK and JNK MAP kinase signaling pathways (Stein et al., 2010, Blood 115:5180-90).

The results below show that the anti-HLA-DR antibody IMMU-114 or hL243γ4P can deplete all subsets of APCs, but not T cells, from human peripheral blood mononuclear cells (PBMCs), including myeloid DCs (mDCs), plasmacytoid DCs (pDCs), B cells and monocytes. In the absence of other human cells or complement, purified mDCs or pDCs were still killed efficiently by IMMU-114, suggesting that IMMU-114 depletes these APCs independently of antibody-dependent cellular cytotoxicity (ADCC) or complement-dependent cytotoxicity (CDC). Furthermore, IMMU-114 suppressed the proliferation of allo-reactive T cells in mixed leukocyte cultures, yet preserved CMV-specific, CD8⁺ memory T cells. These results demonstrate the potential of IMMU-114 as a novel agent for suppressing or preventing allogeneic or xenogeneic immune response, without alteration of preexisting anti-viral immunity.

Methods

Antibodies—

IMMU-114 (hL243γ4p, U.S. Pat. No. 7,612,180) and labetuzumab (hMN-14, U.S. Pat. No. 6,676,924) were prepared as described. Rituximab was purchased from IDEC Pharmaceuticals Corp. (San Diego, Calif.). Commercially available antibodies were obtained from Miltenyi Biotec (Auburn, Calif.):FITC-conjugated antibody to BDCA-2 (AC144), PE-conjugated antibodies to CD19 (LT19) and CD14 (TÜK4), and allophycocyanin (APC)-conjugated antibodies to BDCA-1 (AD5-8E7), BDCA-2 (AC144), and BDCA-3 (AD5-14H12).

Purification of Myeloid and Plasmacytoid DCs from PBMCs—

PBMCs were isolated from the buffy coats of healthy donors by standard density-gradient centrifugation over FICOLL-PAQUE™ (Lonza, Walkersville, Md.). MACS® kits (Miltenyi Biotec) were used to purify DC subsets from PBMCs. mDC1 cells were purified from PBMCs by depleting CD19⁺ B cells, followed by positive enrichment of BDCA-1⁺ cells. pDCs were purified by depleting all the cells that do not express BDCA-4 antigen. mDC2 cells were purified by enriching BDCA-3⁺ cells.

Flow Cytometric Analysis of APC Subsets in Human PBMCs—

PBMCs from normal donors were treated with IMMU-114 or other antibodies at 37° C., 5% CO₂, for 3 days. Following incubation, the cells were stained with PE-labeled anti-CD14 and anti-CD19, in combination with APC-labeled anti-BDCA-1. After washing, 7-amino-actinomycin D (7-AAD, BD Pharmingen) was added, and the cells were analyzed by flow cytometry using the gating strategy described below. The live PBMCs were gated based on the forward scatter (FSC) and side scatter (SSC) signals. Within the live PBMCs, mDC1 cells were identified as CD14⁻19⁻BDCA-1⁺ cell populations (Dzionek et al., 2000, J Immunol 165:6037-6046). Within the same live PBMCs, the lymphocyte population was analyzed for B cells (CD19⁺SSC^{low}), non-B lymphocytes (primarily T cells) (CD19⁻14⁻SSC^{low}), and monocytes (CD14⁺SSC^{medium}). The live cell fraction of each cell population was quantitated as the percentage of 7-AAD⁻ cells. To measure the frequencies of pDCs and mDC2, PBMCs were stained with PE-labeled anti-CD14 and anti-CD19, in combination with FITC-labeled anti-BDCA-2 and APC-labeled anti-BDCA-3. Within the live PBMCs, mDC2 cells were identified as the CD14⁻19⁻BDCA-3⁺⁺ cell population, whereas pDCs were identified as the CD14⁻19⁻BDCA-2⁺ cell population. Flow cytometry was performed using a FACSCALIBUR® (BD Bioscience) and analyzed with FlowJo software (Tree Star, Inc., Ashland, Oreg.).

T-Cell Proliferation in Allogeneic Mixed Leukocyte Reaction—

PBMCs from different donors were labeled with 1 μM carboxyfluorescein succinimidyl ester (CFSE) following the manufacturer's instructions (Invitrogen, CA). After extensive washings, the cells from two different donors were mixed and incubated for 11 days. The cells were then harvested and analyzed by flow cytometry. The proliferating cells were quantitated by measuring the CFSE^{low} cell frequencies.

Quantitation of CMV-Specific T Cells in Allo-MLR by HLA-A*0201 Pentamer—

PBMCs from a donor with a CMV-specific IFN-γ response were mixed with PBMCs from another donor, irrespective of his/her CMV status, in the presence of IMMU-114 or control antibody hMN-14 at 5 μg/ml. The mixed cells were cultured for 4 days in RPMI 1640 medium with 10% fetal bovine serum (1-13S), followed by addition of 50 U/ml IL-2 and were further cultured for 2 more days. The cells were then harvested and stained with PE-labeled HLA-A*0201 CMV pentamer (ProImmune, Bradenton, Fla.) (Wills et al., 1996, J Virol 70:7569-7579; Pita-Lopez et al., 2009, Immun. Ageing 6:11), followed by washing and staining with PerCp-CD8 (BD Pharmingen). The percentages of CMV pentamer⁺ cells in CD8⁺ T cells were assessed by flow cytometry.

Statistical Analysis—

Paired t-test was used to determine the P values comparing the effects between IMMU-114 and control antibody treatment.

Results

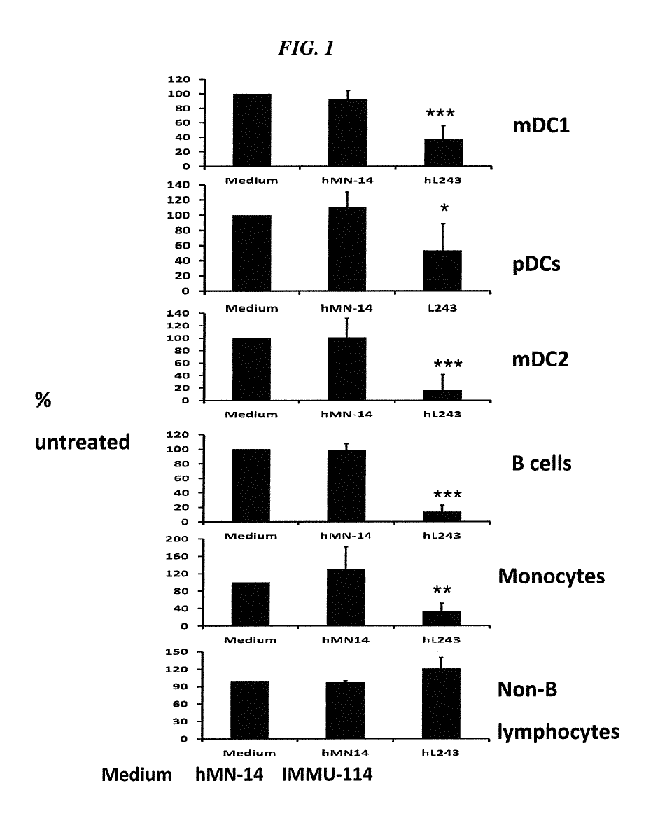

We have demonstrated previously that IMMU-114 efficiently depletes B cells and monocytes, but not T cells or NK cells from human whole blood in vitro (Stein et al., 2010, Blood 115:5180-90). Since both mDCs and pDCs express HLA-DR, IMMU-114 may also deplete these two major subsets of blood DCs. To investigate this, we treated human PBMCs with IMMU-114 or a control antibody (hMN-14 or labetuzumab, humanized anti-CEACAM5 antibody) (Sharkey et al., 1995, Cancer Res. 55(suppl):5935s-5945s) for 3 days, followed by quantitation of various APC subsets in PBMCs by flow cytometry. IMMU-114, but not hMN-14, depleted B cells and monocytes, but not non-B lymphocytes (the majority are T cells) (data not shown), which is consistent with our previous findings in whole blood samples (Stein et al., 2010, Blood 115:5180-90). All blood DC subsets in human PBMCs, including mDC type 1 (mDC1, the major subset of blood mDCs, Dzionek et al., 2000, J Immunol 165:6037-6046), pDCs, and mDC type 2 (mDC2, the minor subset of mDCs, Dzionek et al., 2000, J Immunol 165:6037-6046), were greatly reduced (not shown). As shown in FIG. 1, mDC1 were reduced by 59.2% (P=0.0022, n=6 donors), mDC2 by ˜85% (P<0.01, n=7 donors), B cells by 86.2% (P<0.001, n=6 donors), and monocytes by 74.7% (P=0.01139, n=6 donors), whereas non-B lymphocytes were not reduced. These results demonstrate that IMMU-114 can deplete all APC subsets in human PBMCs.

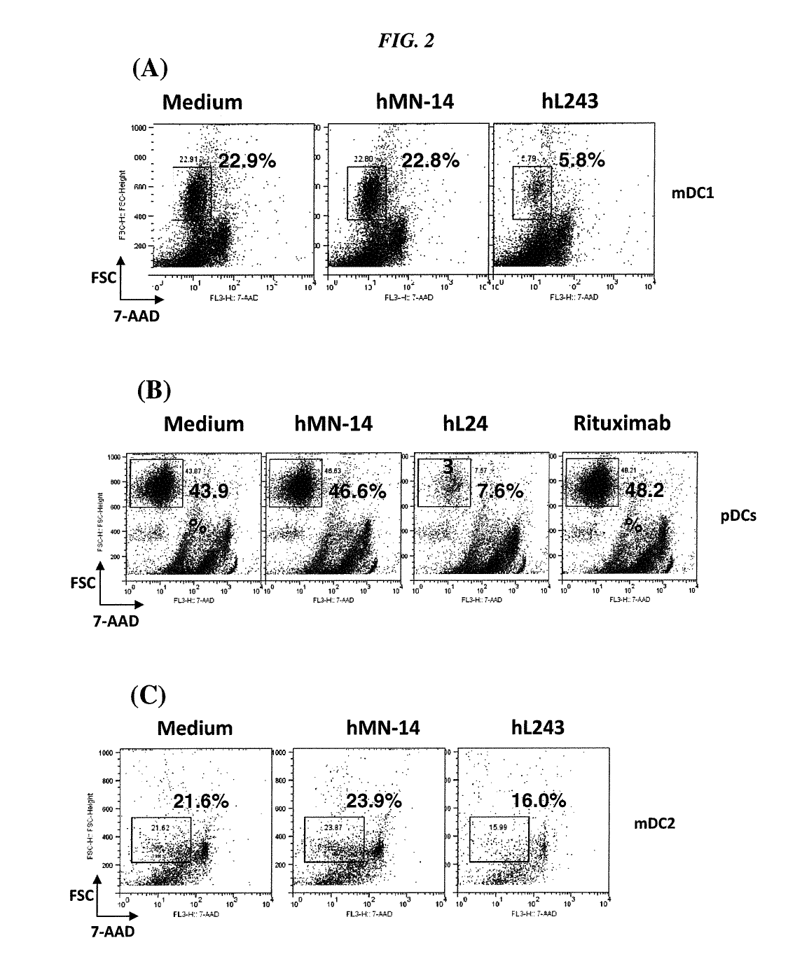

We next determined whether the depletion of APC subsets by IMMU-114 is direct. We isolated mDC1, mDC2, and pDCs from human PBMCs by MACS® selection and treated these purified cells for 2 days with IMMU-114 or control antibody, in the absence of any other cell types or human complement. Cytotoxicity was evaluated by 7-AAD staining and flow cytometry (Klangsinsirikul et al., 2002, Blood 99:2586-2591). In the absence of PBMCs or any other cells, IMMU-114 could still efficiently kill purified mDC1 (FIG. 2A), pDCs (FIG. 2B), or mDC2 (FIG. 2C). These results suggest that IMMU-114 exerts its cytotoxicity on APC subsets through direct action, independent of ADCC or CDC mechanisms.

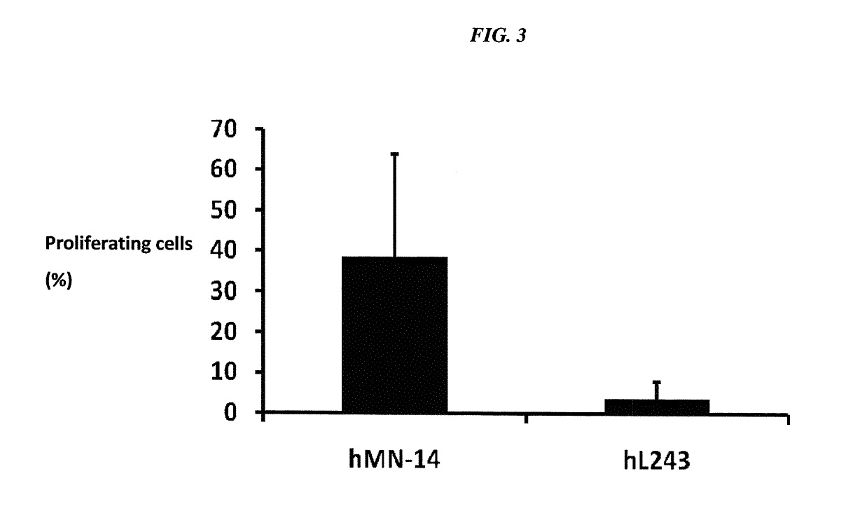

We investigated if the depletion of all APC subsets in PBMCs by IMMU-114 could be translated into reduced allo-proliferation of T cells. We mixed CFSE-labeled PBMCs from two different donors and maintained the cells in culture for 11 days in the presence of IMMU-114 or control antibody, hMN-14. The proliferating allo-reactive T cells were identified based on the CFSE dilution. The allo-MLR treated with the isotype control antibody, hMN-14 (anti-CEACAM5), underwent robust T-cell proliferation characterized by ˜50% of T cells with CFSE dilution. In contrast, T-cell proliferation was only detected in ˜5% of cells in the allo-MLR treated with IMMU-114 (not shown). Statistical analysis of a total of 10 stimulator/responder combinations showed a significant reduction (P<0.01) in T-cell proliferation in IMMU-114-treated allo-MLR (FIG. 3). These data demonstrate a strong inhibitory effect of IMMU-114 on allogeneic T-cell proliferation.

IMMU-114 does not affect T cells, while depleting all subsets of APCs (FIG. 1). This unique property suggests that IMMU-114 does not affect CMV-specific memory T cells. To verify this, we performed a 6-day allo-MLR culture, in which the responder PBMCs were from a CMV-positive, HLA-A*0201 donor, and the stimulator PBMCs were from another donor, irrespective of CMV status. CMV-specific CD8⁺ T cells were determined by staining the cells with HLA*A0201 CMV pentamer. As expected, CMV-specific CD8⁺ T cells were not altered by IMMU-114 treatment (not shown). This result shows that pathogen-specific memory T-cell immunity, such as CMV-specific memory T cells, is not compromised by IMMU-114 treatment.

The results above obtained with samples from four donors showed that hL243 reduced pDCs by about 50%, but the decrease was not statistically significant (P=0.1927). PBMCs from six additional donors were further tested for the effect of hL243 or other antibodies on the survival of pDCs and the HLA-DR⁺ pDC subset. hL243, but not hLL1, depleted plasmacytoid DCs in human PBMCs (data not shown). Human PBMCs were incubated with different mAbs or control at 5 μg/ml, in the absence or presence of GM-CSF (280 U/ml) and IL-3 (5 ng/ml). 3 days later, the cells were stained with APC-labeled BDCA-2 antibody and PerCp-labeled HLA-DR antibody. pDCs were defined as BDCA-2+ cells. hL243 (P=0.0114) but not hLL1 (P=0.5789) or other control antibodies produced a statistically significant decrease in pDCs (BDCA-2⁺) in the absence of GM-CSF and IL-3 (not shown). hL243 (P=0.0066) but not hLL1 (P=0.4799) or other control antibodies produced a statistically significant decrease in HLA-DR⁺ pDCs in the absence of GM-CSF and IL-3 (not shown). Neither hL243 (P=0.1250) nor hLL1 (P=0.2506) or other control antibodies produced a statistically significant decrease in pDCs in the presence of GM-CSF and IL-3 (not shown). hL243 (P=0.0695) but not hLL1 (P=0.2018) or other control antibodies produced a statistically significant decrease in HLA-DR⁺ pDCs in the presence of GM-CSF and IL-3 (not shown). These results show that hL243, but not hLL1, depletes total pDCs and HLA-DR positive pDCs in human PBMCs. The depletion effects were antagonized by the presence of DC survival cytokines GM-CSF and IL-3.

Conclusions

We have shown that IMMU-114, a humanized anti-HLA-DR IgG4 antibody, can deplete all subsets of APCs efficiently, including mDC1, pDC, mDC2, B cells, and monocytes, leading to potent suppression of allo-reactive T cell proliferation, yet preserves CMV-specific, CD8⁺ memory T cells. These findings show that IMMU-114 could be a novel therapeutic agent for suppressing or preventing allogeneic or xenogeneic immune response, by depletion of all subsets of APCs. In comparison with other immunosuppressive antibodies in current use, such as alemtuzumab (anti-CD52), IMMU-114 exhibits a number of surprising advantages. It depletes all APC subsets, providing maximal depletion of host APCs, whereas alemtuzumab depletes only peripheral blood DCs (Buggins et al., 2002, Blood 100:1715-1720). IMMU-114 does not affect T cells, leading to the preservation of pathogen-specific memory T cells, whereas alemtuzumab depletes T cells, leading to reactivation of CMV in allo-HSCT patients. IMMU-114 depletes APC subsets through direct action without the requirement of intact host immunity, whereas alemtuzumab depletes DCs through CDC- and ADCC-mediated mechanisms, which require intact host immune effector functions. Pharmacokinetic data in dogs indicate that IMMU-114 is rapidly cleared from the blood within several hours, followed by the clearance of remaining antibody with a half-life of ˜2 days (not shown), from which the half-life of IMMU-114 in humans is predicted to be 2-3 days according to the allometric scaling of an immunoglobulin fusion protein described by Richter et al. (Drug Metab Dispos 27:21-25, 1999). In contrast, alemtuzumab clears with a half-life of 15-21 days, and the blood concentration at a lympholytic level persists for up to 60 days in patients, resulting in the depletion of donor T cells after transplantation (Morris et al., 2003, Blood 102:404-406; Rebello et al., 2001, Cytotherapy 3:261-267). Thus, donor T cells are expected to be less influenced by IMMU-114 than by alemtuzumab, allowing the donor T cell-mediated third-party immunity to be maximally preserved.

### Example 2

### Anti-HLA-DR Antibody Blocks Allogeneic Immune Response

The effect of an exemplary humanized anti-HLA-DR monoclonal antibody, IMMU-114, on the allogeneic immune response was investigated in vitro. Responder peripheral blood mononuclear cells (PBMCs) were co-cultured with inactivated self (Self) or allogeneic (Allo) stimulator PBMCs in the presence of control antibody or IMMU-114. Thymidine incorporation rates were then measured. Phenotypic changes in PBMCs and the intracellular Th1-type cytokines, IL-2, IFN-γ, and TNF-α were analyzed by flow cytometry. The concentrations of IL-2, IFN-γ, and TNF-α in the MLR culture medium were measured. Thymidine incorporation rates at a 1:1 responder/stimulator ratio of Allo, Allo+IMMU-114, Self, and Self+IMMU-114 were 22080.7±602.4, 2254.5±118.1, 1284.0±227.8, and 494.5±27.5 cpm, respectively (P=0.038). IMMU-114 decreased the frequencies of HLA-DR-expressing CD16⁺56⁺ NK cells, CD19⁺ B cells, and CD3⁺25⁺ activated T cells. Intracellular cytokine assay and measurement of Th1-type cytokines in the MLR culture medium revealed that IMMU-114 significantly decreased the titers of IL-2, IFN-γ, and TNF-α. IMMU-114 significantly suppresses the allogeneic immune response in vitro, partly through inhibition of the Th1 response.

Materials and Methods

The anti-HLA-DR (MHC class II) humanized monoclonal antibody, IMMU-114, was prepared as disclosed in U.S. Pat. No. 7,612,180, the Examples section of which is incorporated herein by reference. The anti-human IgG4 control antibody (HCA050A) was from AbD Serotec (Oxford, UK). Both were used at a concentration of 10 nM in each experiment.

Human Peripheral Blood Mononuclear Cells (PBMCs)

Human peripheral blood mononuclear cells (PBMCs) were collected from heparinized whole blood of healthy volunteers by Ficoll-Hypaque separation. Stimulator PBMCs were inactivated by 30 Gy of irradiation.

Culture of PBMCs

For flow cytometry and enzyme-linked immunoassay (ELISA), 1×10⁶ responder PBMCs were co-cultured with the same number of stimulator PBMCs in 1 ml of AIM V® Medium (LifeTechnologies, Carlsbad, Calif.) in a 24-well plate at 37° C. under 5% CO₂ for 6 days in the presence of control antibody or IMMU-114. The culture medium was collected and analyzed for cytokine concentration by ELISA. Responder PBMCs were collected and analyzed for phenotypic changes by flow cytometry.

In Vitro Mixed Lymphocyte Reaction (MLR)

For the thymidine incorporation assay, 2×10⁵ responder PBMCs were co-cultured with stimulator PBMCs at a responder to stimulator ratio of 1:1, 1:2, 1:4, and 1:8 in 100 μl of AIM V® medium in a 96-well plate at 37° C. After 5 days of culture, the cells were pulsed with 10 μCi/well [³H]thymidine and further cultured overnight. Then, a thymidine incorporation assay was performed. Responder PBMCs were co-cultured with allogeneic stimulator PBMCs (Allo) or self stimulator-PBMCs (Self) in the presence of 10 nM control antibody or IMMU-114. The experiments were repeated 10 times.

CFSE-MLR

Carboxyfluorescein diacetate succinimidyl ester (CFSE; Molecular Probes Inc., Eugene, Oreg.) MLR was performed as described previously (Tanaka et al., Immunol Invest 2004; 33:309-324). Briefly, 2×10⁶ responder cells were incubated with 5 μM CFSE at 37° C. for 15 min. The reaction was then terminated by adding phosphate-buffered saline (PBS) containing 2% fetal calf serum. After two washes with PBS, the responder cells were co-cultured with the same number of stimulator cells in 2 mL of AIM V® medium in a 24-well plate at 37° C. After 6 days of culture, the cells were collected and stained with anti-CD4-PE (RPA-T4, BD, Franklin Lakes, N.J.) or anti-CD8-PE (RPA-T8, BD) antibodies, and then analyzed using a FACSCALIBUR® (BD). Proliferative CD4+ or CD8+ T cells were visualized at a low intensity of CFSE fluorescence. Responder cells were co-cultured with self stimulators, allogeneic stimulators with control antibody, or allogeneic stimulators with IMMU-114. The experiments were repeated 5 times.

Flow Cytometry

Antibodies against the following antigens were used in this study: CD3 FITC, CD16+56 PE, CD4 FITC, CD8 PE, CD19 PE, CD25 PE, HLA-DR APC, CD14 FITC, and CD11c FITC. All the antibodies were purchased from BD (Becton, Dickinson and Co., Franklin Lakes, N.J.). For phenotypic analyses, cells were collected from 24-well plates, and washed twice with PBS. The cells were stained with various antibodies for 30 min at room temperature, washed twice with PBS, and analyzed using a FACSCALIBUR®. Experiments were repeated 6 times. To investigate the effect of IMMU-114 on resting PBMCs, freshly isolated PBMCs were cultured in the presence of control antibody or IMMU-114 in 24 well-plates for 6 days. The experiments were repeated 5 times.

Intracellular cytokine assay was performed using an Intracellular Cytokine Staining Starter Kit-Human (BD). Briefly, 5×10⁶ human PBMCs were cultured in 6-well plates in the presence of Control antibody or IMMU-114. Then 10 μL of Leukocyte Activation Cocktail was added, and the cells were cultured at 37° C. under 5% CO₂ for 4 hours. Finally the cells were washed twice with ice-cold PBS, and analyzed by flow cytometry. The experiments were repeated 4 times.

Enzyme-Linked Immunosorbent Assay (ELISA)

Quantitative analyses for interleukin (IL)-2, interferon (IFN)-γ, and tumor necrosis factor (TNF)-α were performed by using QUANTIKINE® (R&D Systems, Minneapolis, Minn.). Briefly, 200 μL of culture medium from the mixed lymphocyte reaction was transferred to a microplate and treated with 50 μL of RD1F buffer. After 2 hours of incubation at room temperature, the microplate was washed three times, 200 μL of antibody-conjugate was added, and incubation was continued for one hour. The microplate was then washed 4 times and 200 μL of Substrate Solution was added, followed by incubation for 20 min. Then 50 μL of Stop Solution was added, and the concentration was determined using a microplate reader at 450 nm. The experiments were repeated 6 times.

Statistics

Data were expressed as means±standard deviation. Data from the two groups were compared using two-sided t-test. Comparison of more than three groups was performed by one-way analysis of variance (ANOVA). All statistical analyses were performed with GraphPad Prism ver 5.0 (La Jolla, Calif.). Differences at P<0.05 were considered significant.

Results

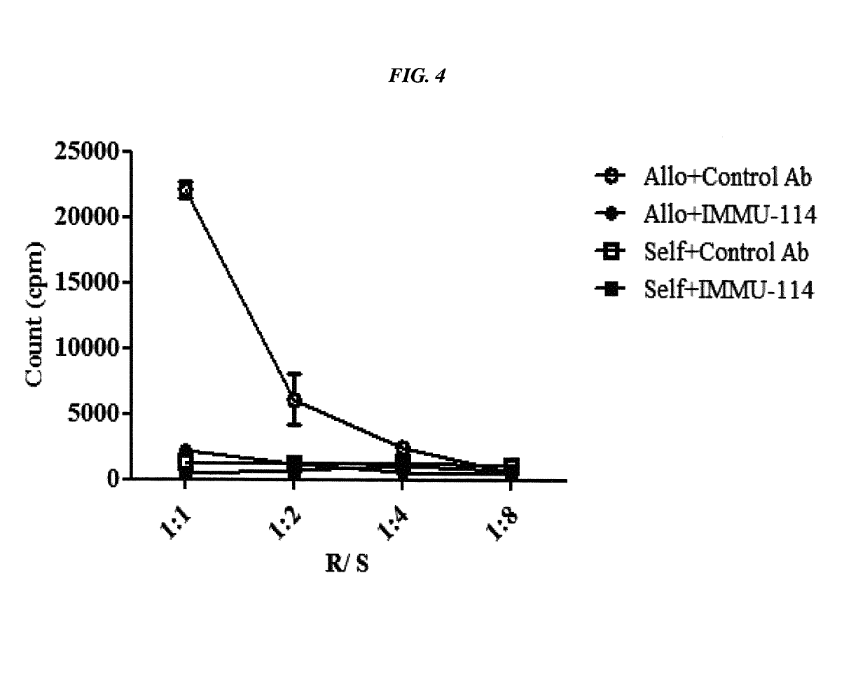

Thymidine incorporation rates after 6 days culture of allogeneic MLR are shown in FIG. 4. Thymidine incorporation rates at a 1:1 responder to stimulator ratio for Allo+control antibody, Allo+IMMU-114, Self+control antibody, and Self+IMMU-114 were 22080.7±602.4, 2254.5±118.1, 1284.0±227.8, and 494.5±27.5 cpm, respectively (P=0.038; one-way ANOVA). Those at a 1:2 ratio were 6106.3±1955.2, 1174.0±203.6, 1270.7±243.0, and 762.7±115.1 cpm, respectively (P=0.046; one-way ANOVA). Those at a 1:4 ratio were 2430.0±48.1, 604.7±71.8, 1319.0±155.6, and 1105.0±123.0 cpm, respectively (P=0.024; one-way ANOVA) and those at a 1:8 ratio were 571.0±216.2, 478.7±80.4, 1075.5±149.2, and 655.0±67.9 cpm, respectively (P=0.1125). Thus, thymidine incorporation rates for Allo+control antibody were significantly higher than those for other three groups at responder to stimulator ratios of 1:1, 1:2, and 1:4. IMMU-114 efficiently inhibited the allogeneic MLR.

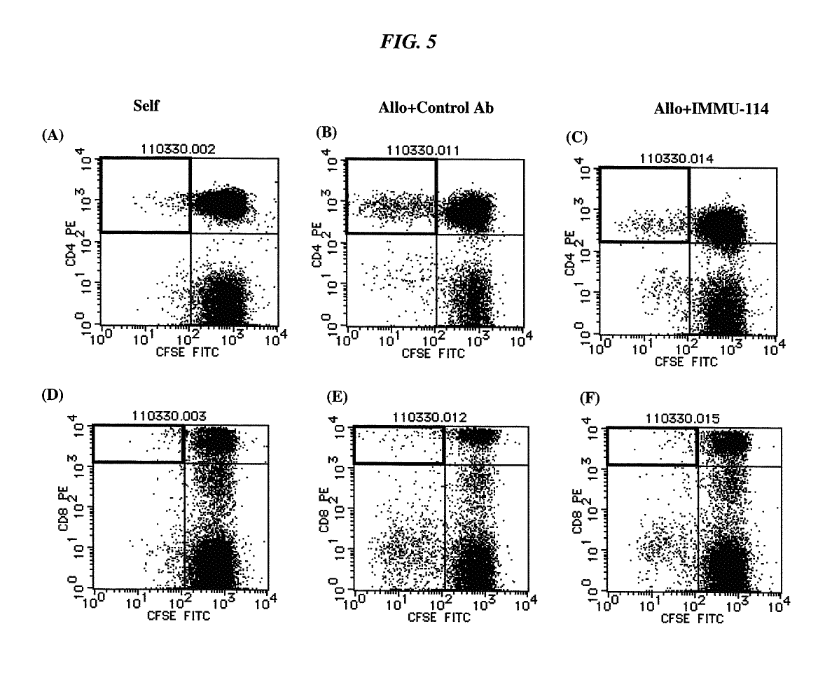

FIG. 5 shows representative results of allogeneic CFSE-MLR. The frequencies of antigen-specific proliferating CD4⁺ T cells among control antibody and IMMU-114 treated cells were 6.4% and 1.4%, respectively, and those of CD8⁺ T cells were 0.9% and 0.4%, respectively. A summary of results with CFSE-MLR is shown in Table 2. Statistical analysis was performed by one-way ANOVA.

TABLE 2
Summary of CFSE-MLR Results

| | Anti-self | Control Ab (%) | IMMU-114 | P-value |
|---|---|---|---|---|
| CD4+ T cells (%) | 1.5 ± 0.8 | 4.9 ± 1.8 | 2.1 ± 1.1 | 0.050 |
| CD8+ T cells (%) | 1.1 ± 0.3 | 1.1 ± 0.5 | 0.8 ± 0.4 | 0.695 |

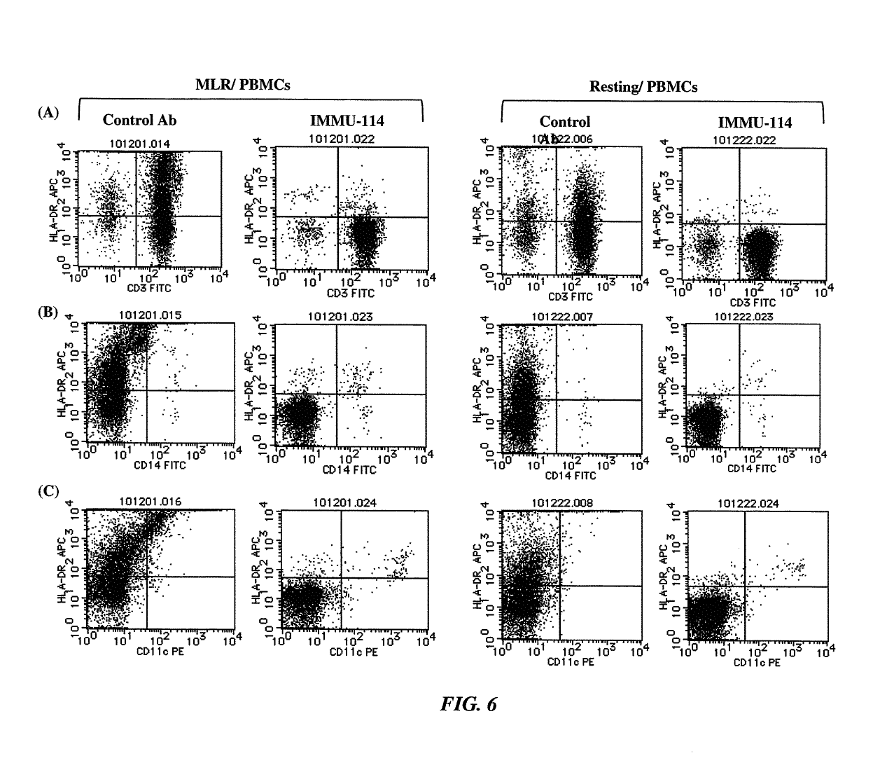

The phenotypic changes in PBMCs of MLR and resting PBMCs treated with control antibody and IMMU-114 were examined (data not shown). Frequencies of CD3⁺ T cells among control antibody and IMMU-114 treated MLR cells, and control antibody and IMMU-114 treated resting PBMCs were 86.9% and 93.3%, and 80.1% and 87.4%, respectively. Those of CD16⁺CD56⁺ NK cells were 8.5% and 3.8%, and 19.0% and 7.3%, respectively. Those of CD3⁺CD16⁺CD56⁺ NKT cells were 0.7% and 0.3%, and 1.3% and 0.7%, respectively. Those of CD4⁺ T cells were 52.6% and 61.6%, and 52.3% and 56.4%, respectively, and those of CD8⁺ T cells were 34.1% and 31.0%, and 34.6% and 33.3%, respectively. Those of CD19⁺ B cells were 3.1% and 0.6%, and 3.6% and 1.0%, respectively, and those of CD3⁺CD25⁺ activated T cells were 5.6% and 4.0%, and 5.5% and 1.4%, respectively. Thus, IMMU-114 did not significantly change the frequency of CD3⁺ T cells, CD4⁺ T cells, and CD8⁺ T cells (not shown). CD16⁺CD56⁺ NK cells, CD3⁺CD16⁺CD56⁺ NKT cells, CD19⁺ B cells, and CD3⁺CD25⁺ activated T cells of MLR and resting PBMCs were significantly depleted by IMMU-114 (not shown). IMMU-114 effectively eliminated HLA class II-DR+ cells (FIG. 6). A summary of the phenotypic changes in PBMCs is shown in the attached Table 3. Statistical analysis was by t test.

To investigate whether IMMU-114 inhibited Th1 cells, intracellular cytokine analysis was performed. PBMCs were cultured in the presence of control antibody or IMMU-114 with 10 of leukocyte activation cocktail at 37° C. under 5% CO₂ for 4 hours. Intracellular IL-2, IFN-γ, and TNF-α were analyzed by flow cytometry (data not shown). The frequencies of IL-2-producing cells among control antibody- and IMMU-114-treated cells were 16.4% and 4.1%, respectively. Those of IFN-γ-producing cells were 10.8% and 5.7%, respectively, and those of TNF-α-producing cells were 16.3% and 2.6%, respectively. A summary of the intracellular cytokine analysis is shown in Table 4. Statistical analysis was performed using two-sided t-test.

TABLE 4
Summary of Intracellular Cytokine Analyses

| | Control Ab (%) | IMMU-114 | P-value |
|---|---|---|---|
| IL-2 (%) | 16.8 ± 1.1 | 4.3 ± 0.8 | <0.001 |
| IFN-γ (%) | 16.4 ± 1.4 | 8.9 ± 0.7 | 0.001 |
| TNF-α | 19.9 ± 0.9 | 7.4 ± 0.5 | <0.001 |

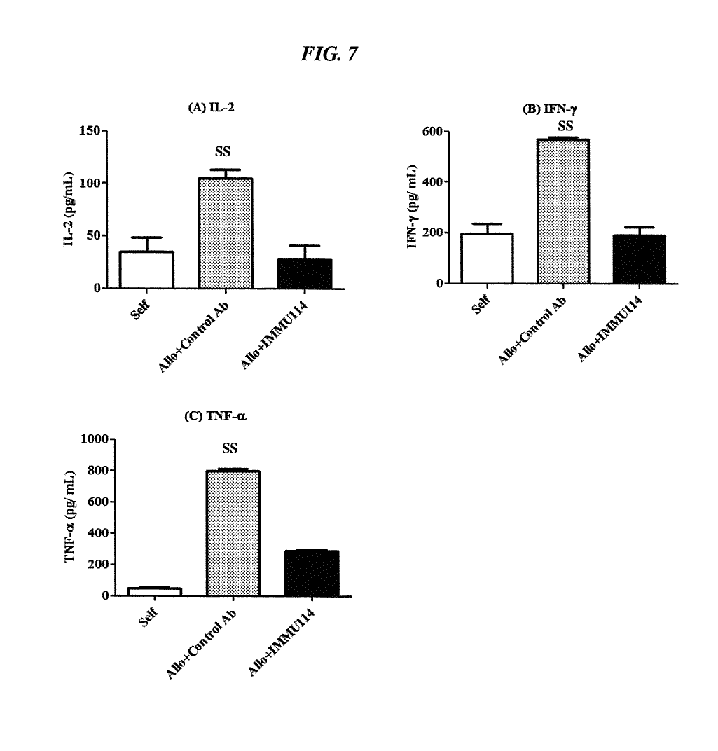

We then evaluated the concentrations of IL-2, IFN-γ, and TNF-α in the in vitro MLR culture medium (FIG. 7). The mean concentrations of IL-2 for Self, Allo+control antibody, and Allo+IMMU-114 were 34.8±14.1, 104.9±8.3, and 28.3±12.5 pg/mL, respectively (P=0.0004; one-way ANOVA). Those for IFN-γ were 196.7±40.6, 570.2±8.6, and 192.2±31.1 pg/mL, respectively (P=0.0001; one-way ANOVA), and those for TNF-α were 46.8±4.7, 796.7±14.9, and 288.3±8.1 pg/mL, respectively (P<0.0001; one-way ANOVA). The suppression of Th1 cytokine production by IMMU-114 was significant.

Discussion

In this study, we showed that the exemplary anti-HLA-DR antibody, IMMU-114, efficiently inhibited the allogeneic immune response in vitro. IMMU-114 is a humanized anti-HLA-DR monoclonal antibody that was initially designed for B-cell malignancies that are refractory to the anti-CD20 monoclonal antibody, rituximab (Stein et al., Blood 2006; 108:2736-2744; Stein et al., Blood 2010; 115:5180-5190). IMMU-114 is a humanized IgG4 form of the murine anti-HLA-DR monoclonal antibody, L243, and recognizes a conformational epitope in the α chain of HLA-DR. Because it is a humanized IgG4 antibody, IMMU-114 has fewer effector-related side effects, and related thereto, its cytotoxic effects are not due to complement-dependent cytotoxicity (CDC) and antibody-dependent cellular cytotoxicity (ADCC), which are the main cytotoxic mechanisms of other therapeutic IgG1 monoclonal antibodies.

To exert its cytotoxicity, IMMU-114 has a dual requirement for HLA-DR expression and activation of MAP kinases by targeted cells (Stein et al., Blood 2010; 115:5180-5190). IMMU-114 induces apoptosis in targeted cells upon binding to HLA-DR, with activation of JNK1/2 and ERK1/2 MAP kinases (Id.). It has been suggested that IMMU-114 kills activated T cells but not resting T cells (Id.). Indeed, the phenotypic changes in PBMCs of MLR and resting PBMCs after 6 days of culture with IMMU-114 differed significantly among HLA-DR-expressing CD16⁺CD56⁺ NK cells, CD3⁺CD16⁺CD56⁺ NKT cells, CD19⁺ B cells, CD3⁺CD25⁺ activated T cells, CD14⁺ macrophages, and CD11c⁺ dendritic cells (Table 3). Exclusive killing of activated cells expressing HLA-DR may be beneficial in the setting of transplantation, because host effector cells are donor antigen-specific and their proliferation is pivotal for eliciting rejection (Ford et al., J Exp Med 2007; 204: 299-309).

In both direct and indirect recognition, host CD4⁺ T cells recognize allogeneic donor antigens and HLA-class II complex, and are activated to assist the subsequent immune response. IMMU-114 eliminated HLA-class II-DR-expressing cells and inhibited alloantigen-specific lymphocyte proliferation (FIG. 4). The inhibitory effect of IMMU-114 was sufficiently strong to decrease the thymidine incorporation rate to the level of the control group. At the same time, as shown in FIG. 5, IMMU-114 inhibited the proliferation of alloantigen-specific CD4+ T cells, and tended to suppress the proliferation of CD8+ T cells, but without statistical significance. Because CD8+ T cells are MHC class I-restricted, it appears that IMMU-114 killed HLA-class II-DR-expressing cells and inhibited the activation of CD4+ T cells, and that the inactivated T cells were unable to produce Th1 cytokines, including IL-2, IFN-γ, and TNF-α.

It has been thought that Th1 cells are involved in graft rejection, whereas Th2 cells play a role in graft protection (D'Elios et al., Kidney Int 1997; 51:1876-1884). However, this simple paradigm has been challenged by many studies (e.g., Tay et al., Curr Opin Organ Transplant 2009; 14:16-22), Our present intracellular cytokine assay revealed that IMMU-114 suppressed the development of IL-2-, IFN-γ-, and TNF-α-producing Th1 cells, and suppression of the development of Th1 cells was confirmed by measurement of Th1-type cytokines in the MLR culture supernatant by ELISA (FIG. 7). Although we did not evaluate Th2- or Th17-type cytokines, the inhibitory effect of IMMU-114 on allo-specific lymphocyte proliferation is exerted through suppression of the Th1-mediated immune response.

One challenging area in organ transplantation is the pre-sensitization and cross-matching of recipients. Pre-sensitized recipients already possess anti-donor antigens, including anti-class II-DR antigen. Although many de-sensitizing protocols for these recipients have been attempted, only limited success has been achieved (Ford et al., J Exp Med 2007; 204:299-309). The results above indicate that the use of anti-HLA-DR antibodies, such as IMMU-114, is beneficial when performing organ transplantation in pre-sensitized, cross-match-positive recipients.

In conclusion, IMMU-114 suppresses the allogeneic immune response in vitro, in part by inhibiting the generation of Th1-deviated CD4+ T cells and the production of Th1-type cytokines. These results show that IMMU-114 is a useful agent to prevent rejection in organ transplantation or to overcome GVHD in patients undergoing allogeneic stem-cell transplantation. The person of ordinary skill will realize that these results are not limited to IMMU-114, but may be produced by other anti-HLA-DR antibodies.

### Example 3

### Anti-HLA-DR Antibody Suppresses Xenogeneic Immune Response In Vitro

The efficacy of anti-HLA-DR antibodies, such as IMMU-114, in suppressing immune response was also observed with the xenogeneic human to bovine cellular response. Human peripheral mononuclear cells (PBMCs) were co-cultured with inactivated self-PBMCs (Self), bovine PBMCs with control antibody (Xeno), or bovine PBMCs with IMMU-114 (IMMU-114). Cellular responses were investigated by thymidine incorporation assay, CFSE-mixed lymphocyte reaction (MLR), and cytokine production in culture medium. As discussed below, thymidine incorporation rates at a 1:1 responder to stimulator ratio for Xeno+control antibody, Xeno+IMMU-114, Self+control antibody, and Self+IMMU-114 were 14201.3±1968.4, 513.0±49.5, 952.7±128.7, and 423.3±138.8 cpm, respectively (P=0.032). Those at a 1:2 ratio were 6518.0±690.1, 896.6±92.9, 1051.0±123.6, and 736.0±35.6 cpm, respectively (P=0.036). CFSE-MLR demonstrated that the frequencies of CFSE-low, CD4-positive, and CD25-positive activating T cells in Self, Xeno, and IMM-114, were 0.27±0.04%, 3.65±0.53%, and 1.23±0.15%, respectively (P=0.027). Cytokine production in culture medium indicated that IMMU-114 decreased Th-1 type cytokines, including interleukin-2 (IL-2), interferon-γ, and tumor necrosis factor-α. The results demonstrate that IMMU-114 effectively suppresses human to bovine cellular responses and is of use to suppress immune response to xenogeneic organ transplant. The mechanism involves direct inhibition of the interaction between class II HLA-DR-positive cells and CD4+ T cells, and indirect suppression of Th-1 cytokine production.

Materials and Methods

The anti-HLA-DR (MHC class II) humanized monoclonal antibody, IMMU-114, was prepared as disclosed in U.S. Pat. No. 7,612,180, the Examples section of which is incorporated herein by reference. An anti-human IgG4 control antibody (HCA050A) was obtained from AbD Serotec (Oxford, UK). Both were used at a concentration of 10 nM in each experiment.

Human Peripheral Blood Mononuclear Cells (PBMCs)

Human peripheral blood mononuclear cells (PBMCs) were collected from heparinized whole blood of five healthy volunteers by Ficoll-Hypaque separation. Bovine PBMCs were obtained from two Japanese black cattle (ZENNOH, Hokkaido, Japan) and isolated in the same way as human PBMCs. Stimulator PBMCs were inactivated by 30 Gy of irradiation.

Culture of PBMCs

One million responder (human) PBMCs were co-cultured with the same number of stimulator PBMCs in 1 ml of AIM V® Medium (Life Technologies, Carlsbad, Calif.) in a 24-well plate at 37° C. under 5% CO₂ for 6 days in the presence of control antibody (Xeno) or IMMU-114 (IMMU-114). MLR performed between human PBMC-responder and self-PBMC-stimulator was designated as Self. The culture medium was collected and analyzed for cytokine concentration using ELISA. Responder PBMCs were collected and analyzed for phenotypic changes by flow cytometry.

In Vitro Mixed Lymphocyte Reaction (MLR)

For the thymidine incorporation assay, 2×10⁵ responder human PBMCs were co-cultured with stimulator PBMCs at a responder to stimulator ratio of 1:1, 1:2, 1:4, or 1:8 in 100 μl of AIM V® medium in a 96-well plate at 37° C. After 5 days of culture, the cells were pulsed with 10 μCi/well [³H]thymidine and further cultured overnight. Then, a thymidine incorporation assay was performed.

CFSE-MLR

Carboxyfluorescein diacetate succinimidyl ester (CFSE; Molecular Probes Inc., Eugene, Oreg.) MLR was performed as described previously (Tanaka et al., Immunol Invest 2004; 33:309). Briefly, 2×10⁶ responder cells were incubated with 5 μM CFSE at 37° C. for 15 min. The reaction was then terminated by adding phosphate-buffered saline (PBS) containing 2% fetal calf serum. After two washes with PBS, the responder cells were co-cultured with the same number of stimulator cells in 2 ml of AIM V® medium in a 24-well plate at 37° C. After 6 days of culture, the cells were collected and stained with anti-CD4-PE (RPA-T4, BD, Franklin Lakes, N.J.) antibodies, and then analyzed using a FACSCALIBUR® (BD). Proliferative CD4+ cells were visualized at a low intensity of CFSE fluorescence.

Enzyme-Linked Immunosorbent Assay (ELISA)

Quantitative analyses for interleukin-2 (IL-2), interferon-γ (IFN-γ), tumor necrosis factor-α (TNF-α), IL-6, IL-4, and IL-17 were performed using QUANTIKINE® (R&D Systems, Minneapolis, Minn.). Briefly, 200 μl of culture medium from the MLR was transferred to a microplate and treated with 50 μl of RD1F buffer. After 2 h of incubation at room temperature, the microplate was washed three times, 200 μl of antibody-conjugate was added, and incubation was continued for 1 h. The microplate was then washed 4 times, and 200 μl of substrate solution was added, followed by incubation for 20 min. Then 50 μl of stop solution was added, and the concentration was determined using a microplate reader at 450 nm. The experiments were repeated 6 times.

Statistics

Data were expressed as means±standard deviation. Comparison of more than three groups was performed by one-way analysis of variance (ANOVA). All statistical analyses were performed with GraphPad Prism 5.0 (La Jolla, Calif.). Differences at P<0.05 were considered significant.

Results

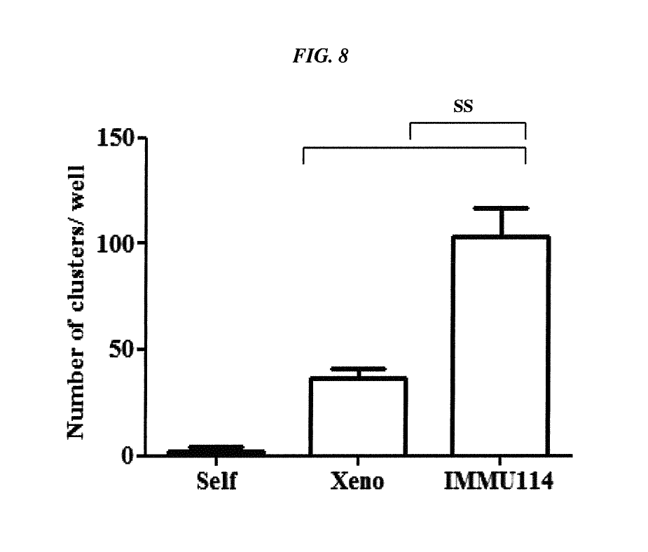

The microscopic appearance of in vitro MLR was examined at 6 days of culture (data not shown). Xenogeneic MLR in the presence of the exemplary anti-HLA-DR antibody IMMU-114 revealed many characteristic cell clusters (not shown). The numbers of cell clusters (FIG. 8) of Self, Xeno+control antibody, and Xeno+IMMU-114 were 2.0±2.1, 36.2±5.0, and 103±13.8, respectively (P=0.0005; one-way ANOVA).

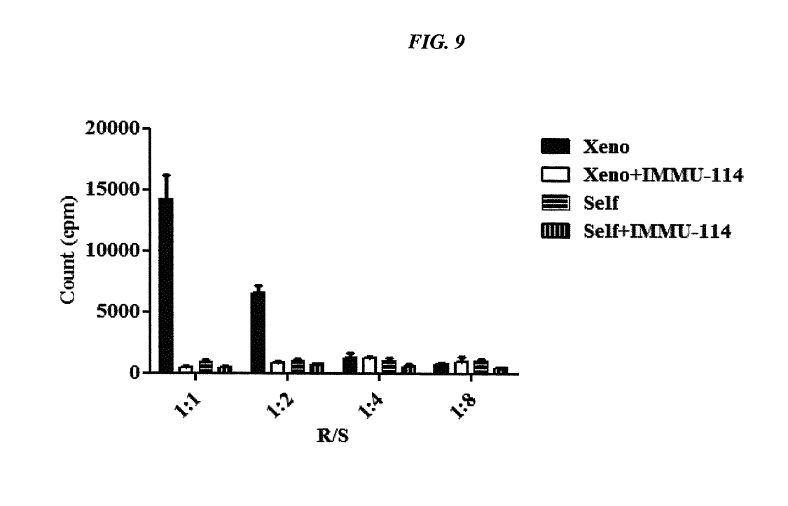

In order to determine whether the exemplary anti-HLA-DR antibody IMMU-114 diminished human to bovine proliferative xenogeneic responses, bovine and human PBMC were co-cultured with IMMU-114 or an irrelevant antibody. As shown in FIG. 9, IMMU-114 significantly suppressed the human to bovine xenogeneic proliferative response, at 1:1 and 1:2 responder to stimulator ratios.

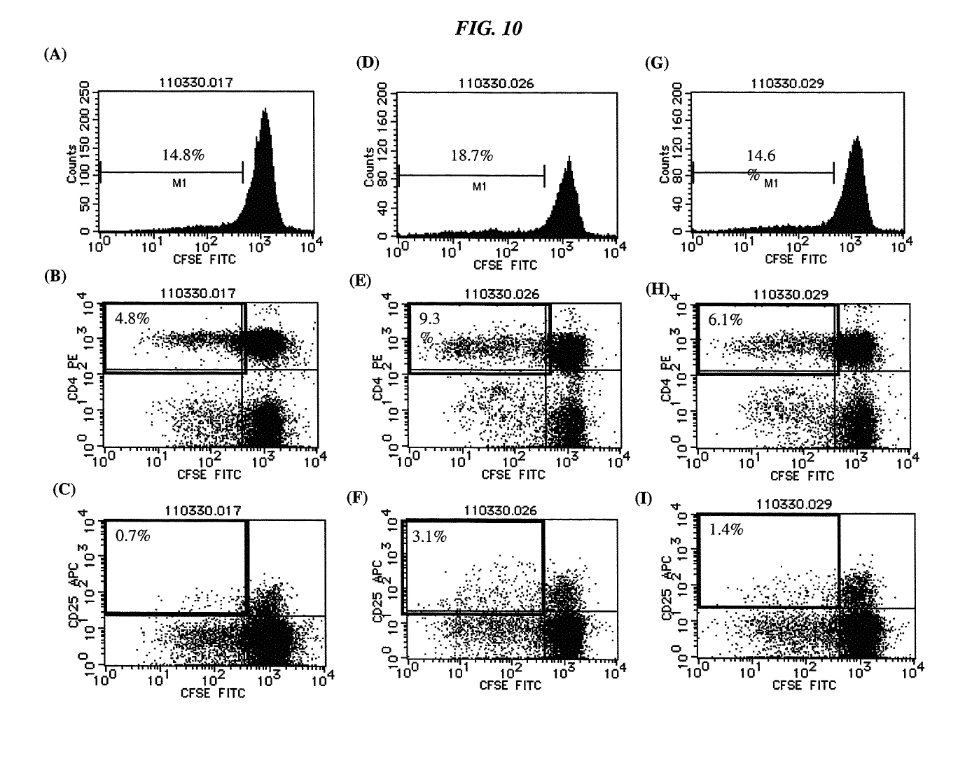

FIG. 10 shows representative results of xenogeneic CFSE-MLR. The frequencies of CFSE-low proliferating cells in Self (FIG. 10A) and IMMU-114 (FIG. 10G) were significantly lower than that in Xeno (FIG. 10D). The frequencies of CFSE-low, activating CDC T cells in Self (FIG. 10B) and IMMU-114 (FIG. 10H) were significantly lower than that in Xeno (FIG. 10E). Also, the frequencies of CFSE-low, CD4-positive, and CD25-positive activating T cells in Self (FIG. 10C) IMMU-114 (FIG. 10I) were significantly lower than that in Xeno (FIG. 10F).

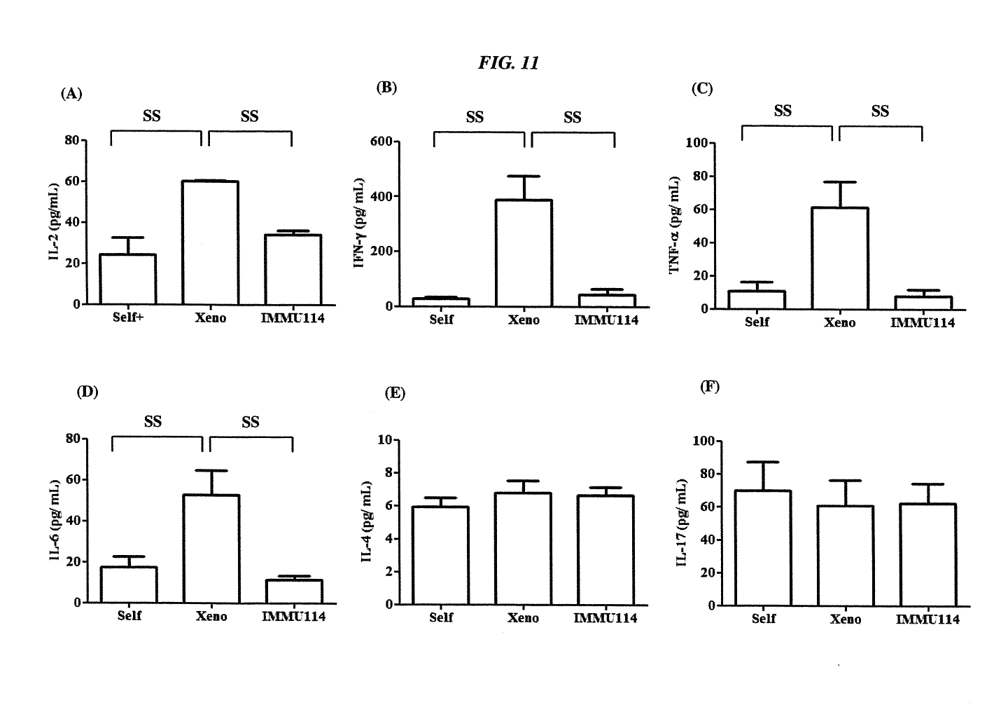

In order to determine whether the exemplary anti-HLA-DR antibody IMMU-114 suppresses cytokine production during human to bovine proliferative xenogeneic responses, the concentrations of IL-2, IFN-γ, TNF-α, IL-6, IL-4, and IL-17 in the in vitro MLR culture medium were measured by ELISA (FIG. 11). The concentrations of IL-2 (FIG. 11A), IFN-γ (FIG. 11B), TNF-α (FIG. 11C), and IL-6 (FIG. 11D) for Self, IMMU-114 were significantly lower than those for Xeno. And there was no significant differences between Self and IMMU-114. On the other hand, there were no significant differences in IL-6 (FIG. 11E) and IL-17 (FIG. 11F) among the three groups.

Discussion

In the present study, we demonstrated that the exemplary anti-HLA-DR antibody IMMU-114 effectively suppressed the human anti-bovine xenogeneic cellular response. Xenogeneic MLR in the presence of IMMU-114 was about 10-fold weaker than that in its absence at a responder to stimulator ratio of 1:1 (FIG. 9). Furthermore, the proportion of xenoantigen-specific T cells was decreased significantly by treatment with IMMU-114 (FIG. 10).

CFSE is transmitted to daughter cells in the process of cell division, and thus its concentration decreases according to the number of cell divisions. The proportion of CFSE-low (M1-population) cells among IMMU-114-treated cells was significantly lower than that of control antibody-treated cells, and was the same as that for the Self-MLR response. Furthermore, CFSE-low, CD4⁺, and CD25⁺ T cells were xenoantigen-specific, activated T cells whose proportion among IMMU-114-treated cells was significantly lower than that among cells treated with control antibody (FIG. 9). However, CD25 is also expressed on regulatory T cells, which can potentially suppress T cell activation. Thus the effect of IMMU-114 on regulatory T cells remains unclear.

Human T-cell responses against xenogeneic antigens are stronger than responses against alloantigens, especially via the indirect pathway (Dorling et al., Eur J Immunol 1996; 26:1378). In the indirect pathway, xenogeneic antigens are processed in human dendritic cells, and xenogeneic antigens are presented with human MHC class II to CD4+ T cells. T-cell suppression by blocking class II MHC and CD4+ T-cell interaction is an attractive way to prolong xenograft survival. Experimentally, it has been shown that suppression of T-cell function prolongs the survival of porcine xenografts (Yamada et al., Nature Med 2005; 11:32).

Tissue injury due to xenograft rejection can be caused by cytokines originating from T cells, which are activated by interaction with xenoantigens (Layton et al., Xenotransplantation 2008; 15:257). IMMU-114 significantly suppressed the production of Th1-cytokines, including IL-2, IFN-γ, and TNF-α, and also the inflammatory cytokine, IL-6. On the other hand, the Th2-cytokine, IL-4, and the Th17-cytokine, IL-17, were not significantly suppressed (FIG. 11).

IMMU-114 deletes MHC-class II HLA-DR-positive cells, including macrophages, some B cells, and NK cells (Stein et al., Leuk Lymphoma 2011; 52:273). The simple 6-day xenogeneic MLR used in the present study effectively generated Th1-type stimulation, which was suppressed by IMMU-114. The effect of IMMU-114 against Th2- and Th17-type T-cell proliferation needs to be clarified.

Chen et al. reported that IMMU-114 might be beneficial for treating graft-versus-host disease (GVHD) (Chen et al., Bone Marrow Transplant 2012, 47:967-80), as IMMU-114 depleted MHC-class II HLA-DR-positive dendritic cells, B cells, and monocytes. Furthermore, IMMU-114 depleted MHC-class II HLA-DR-positive, antigen-specific, alloreactive T cells.

In this study, we have shown that IMMU-114 is a powerful tool for suppressing the T-cell response against xenogeneic antigens, through both direct inhibition of the interaction between MHC class II HLA-DR-positive cells and CD4+ T cells, and indirect suppression of proinflammatory cytokine production.

### Example 4

### Anti-HLA-DR Antibody Suppresses Allogeneic Immune Response In Vivo

IMMU-114 is an anti-HLA class II-DR humanized monoclonal antibody that depletes HLA-DR positive cells by inducing apoptosis, not by ADCC nor CDCC. The effect of IMMU-114 on transplantation was investigated in monkey kidney transplantation (KT) model.

Male crab-eating monkeys, weighing 3-4 kg, were divided into two groups: Control-G (n=2) and IMMU-G (n=2). In control-G, KT was performed without immunosuppressions. In IMMU-G, 3 mg/kg IMMU-114 was given intravenously to monkeys on day −7 and 0. In KT, donor-kidney was transplanted intraabdominal cavity and recipient's ureters were ligated bilaterally. Serum Creatinine (Cr) and Blood urea nitrogen (BUN) were measured preoperatively (Pre) and day 7.

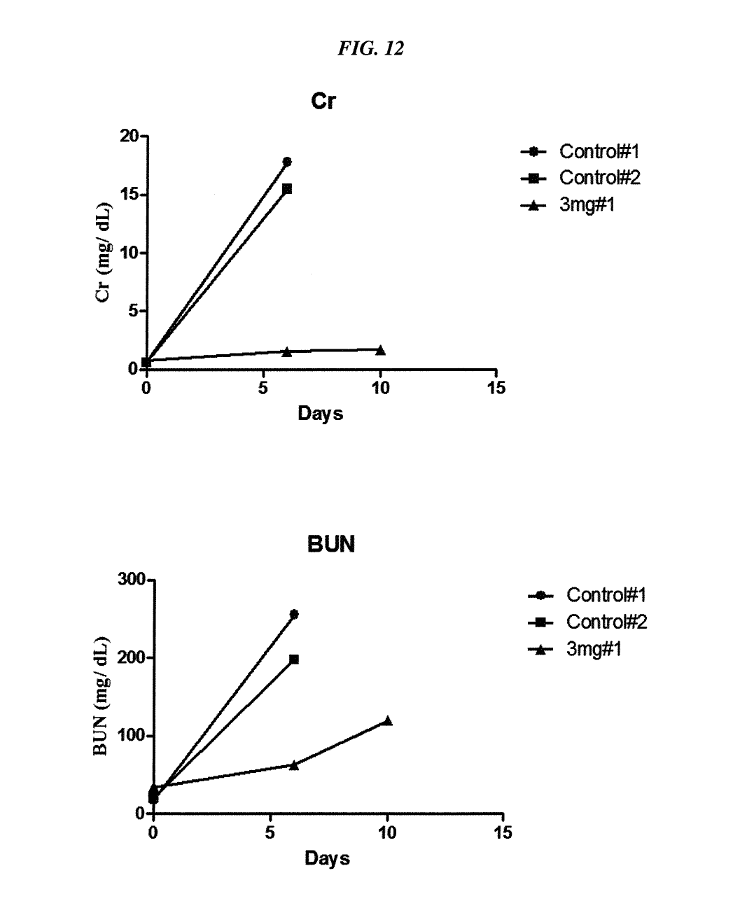

No side effects associated with IMMU-114 infusion were observed. Monkeys in Control-G died on day 6 and 7. And the monkeys in IMMU-G died on day 10. Mean Cr-values at Pre and day 7 of Control-G were 0.6 mg/dl and 16.6 mg/dl and those of IMMU-G were 0.7 mg/dl and 1.6 mg/dl (FIG. 12). Mean BUN-values at Pre and day 7 of Control-G were 19.3 mg/dl and 226.7 mg/dl and those of IMMU-G were 34.7 mg/dl and 63.0 mg/dl (FIG. 12). Mean Cr- and BUN-values at day 10 of IMMU-G were 1.7 mg/dl and 119.8 mg/dl (FIG. 12). Biopsy at day 6 of Control-G showed the severe acute rejection. Biopsy of IMMU-G showed mild (day 6) and severe (day 10) acute rejection.

In conclusion IMMU-114 at a dose of 3 mg/kg had a suppressive effect on allogeneic immune response and a positive effect on graft-survival in the in vivo monkey KT model.

### Example 5

### Preparation of DNL™ Constructs

DDD and AD Fusion Proteins

The technique can be used to make dimers, trimers, tetramers, hexamers, etc. comprising virtually any antibody, antibody fragment, cytokine or other effector moiety. For certain preferred embodiments, antibodies, cytokines, toxins or other protein or peptide effectors may be produced as fusion proteins comprising either a dimerization and docking domain (DDD) or anchoring domain (AD) sequence. Although in preferred embodiments the DDD and AD moieties may be joined to antibodies, antibody fragments, cytokines or other effectors as fusion proteins, the skilled artisan will realize that other methods of conjugation exist, such as chemical cross-linking, click chemistry reaction, etc.

The technique is not limiting and any protein or peptide of use may be produced as an AD or DDD fusion protein for incorporation into a DNL™ construct. Where chemical cross-linking is utilized, the AD and DDD conjugates may comprise any molecule that may be cross-linked to an AD or DDD sequence using any cross-linking technique known in the art. In certain exemplary embodiments, a dendrimer or other polymeric moiety such as polyethyleneimine or polyethylene glycol (PEG), may be incorporated into a DNL™ construct, as described in further detail below.

For different types of DNL™ constructs, different AD or DDD sequences may be utilized. Exemplary DDD and AD sequences are provided below.

DDD1:
(SEQ ID NO: 45)
SHIQIPPGLTELLQGYTVEVLRQQPPDLVEFAVEYFTRLREARA
DDD2:
(SEQ ID NO: 46)
CGHIQIPPGLTELLQGYTVEVLRQQPPDLVEFAVEYFTRLREARA
AD1:
(SEQ ID NO: 47)
QIEYLAKQIVDNAIQQA
AD2:
(SEQ ID NO: 48)
CGQIEYLAKQIVDNAIQQAGC

The skilled artisan will realize that DDD1 and DDD2 comprise the DDD sequence of the human RIIα form of protein kinase A. However, in alternative embodiments, the DDD and AD moieties may be based on the DDD sequence of the human RIα form of protein kinase A and a corresponding AKAP sequence, as exemplified in DDD3, DDD3C and AD3 below.

DDD3
(SEQ ID NO: 49)
SLRECELYVQKHNIQALLKDSIVQLCTARPERPMAFLREYFERLEKEEA
K
DDD3C
(SEQ ID NO: 50)
MSCGGSLRECELYVQKHNIQALLKDSIVQLCTARPERPMAFLREYFERL
EKEEAK
AD3
(SEQ ID NO: 51)
CGFEELAWKIAKMIWSDVFQQGC

Expression Vectors

The plasmid vector pdHL2 has been used to produce a number of antibodies and antibody-based constructs. See Gillies et al., J Immunol Methods (1989), 125:191-202; Losman et al., Cancer (Phila) (1997), 80:2660-6. The di-cistronic mammalian expression vector directs the synthesis of the heavy and light chains of IgG. The vector sequences are mostly identical for many different IgG-pdHL2 constructs, with the only differences existing in the variable domain (VH and VL) sequences. Using molecular biology tools known to those skilled in the art, these IgG expression vectors can be converted into Fab-DDD or Fab-AD expression vectors. To generate Fab-DDD expression vectors, the coding sequences for the hinge, CH2 and CH3 domains of the heavy chain are replaced with a sequence encoding the first 4 residues of the hinge, a 14 residue Gly-Ser linker and the first 44 residues of human RIIα (referred to as DDD1). To generate Fab-AD expression vectors, the sequences for the hinge, CH2 and CH3 domains of IgG are replaced with a sequence encoding the first 4 residues of the hinge, a 15 residue Gly-Ser linker and a 17 residue synthetic AD called AKAP-IS (referred to as AD1), which was generated using bioinformatics and peptide array technology and shown to bind RIIα dimers with a very high affinity (0.4 nM). See Alto, et al. Proc. Natl. Acad. Sci., U.S.A (2003), 100:4445 50.

Two shuttle vectors were designed to facilitate the conversion of IgG-pdHL2 vectors to either Fab-DDD1 or Fab-AD1 expression vectors, as described below.

Preparation of CH1

The CH1 domain was amplified by PCR using the pdHL2 plasmid vector as a template. The left PCR primer consisted of the upstream (5′) end of the CH1 domain and a SacII restriction endonuclease site, which is 5′ of the CH1 coding sequence. The right primer consisted of the sequence coding for the first 4 residues of the hinge (PKSC, SEQ ID NO:98) followed by four glycines and a serine, with the final two codons (GS) comprising a Bam HI restriction site. The 410 bp PCR amplimer was cloned into the PGEMT® PCR cloning vector (PROMEGA®, Inc.) and clones were screened for inserts in the T7 (5′) orientation.

A duplex oligonucleotide was synthesized to code for the amino acid sequence of DDD1 preceded by 11 residues of the linker peptide, with the first two codons comprising a BamHI restriction site. A stop codon and an EagI restriction site are appended to the 3′ end. The encoded polypeptide sequence is shown below.

(SEQ ID NO: 52)
GSGGGGSGGGGSHIQIPPGLTELLQGYTVEVLRQQPPDLVEFAVEYFTR
LREARA

Two oligonucleotides, designated RIIA1-44 top and RIIA1-44 bottom, which overlap by 30 base pairs on their 3′ ends, were synthesized and combined to comprise the central 154 base pairs of the 174 bp DDD1 sequence. The oligonucleotides were annealed and subjected to a primer extension reaction with Taq polymerase. Following primer extension, the duplex was amplified by PCR. The amplimer was cloned into PGEMT® and screened for inserts in the T7 (5′) orientation.

A duplex oligonucleotide was synthesized to code for the amino acid sequence of AD1 preceded by 11 residues of the linker peptide with the first two codons comprising a BamHI restriction site. A stop codon and an EagI restriction site are appended to the 3′ end. The encoded polypeptide sequence is shown below.

(SEQ ID NO: 53)
GSGGGGSGGGGSQIEYLAKQIVDNAIQQA

Two complimentary overlapping oligonucleotides encoding the above peptide sequence, designated AKAP-IS Top and AKAP-IS Bottom, were synthesized and annealed. The duplex was amplified by PCR. The amplimer was cloned into the PGEMT® vector and screened for inserts in the T7 (5′) orientation.

Ligating DDD1 with CH1

A 190 bp fragment encoding the DDD1 sequence was excised from PGEMT® with BamHI and NotI restriction enzymes and then ligated into the same sites in CH1-PGEMT® to generate the shuttle vector CH1-DDD1-PGEMT®.

Ligating AD1 with CH1

A 110 bp fragment containing the AD1 sequence was excised from PGEMT® with BamHI and NotI and then ligated into the same sites in CH1-PGEMT® to generate the shuttle vector CH1-AD1-PGEMT®.

Cloning CH1-DDD1 or CH1-AD1 into pdHL2-Based Vectors

With this modular design either CH1-DDD1 or CH1-AD1 can be incorporated into any IgG construct in the pdHL2 vector. The entire heavy chain constant domain is replaced with one of the above constructs by removing the SacII/EagI restriction fragment (CH1-CH3) from pdHL2 and replacing it with the SacII/EagI fragment of CH1-DDD1 or CH1-AD1, which is excised from the respective pGemT shuttle vector.

Construction of h679-Fd-AD1-pdHL2

h679-Fd-AD1-pdHL2 is an expression vector for production of h679 Fab with AD1 coupled to the carboxyl terminal end of the CH1 domain of the Fd via a flexible Gly/Ser peptide spacer composed of 14 amino acid residues. A pdHL2-based vector containing the variable domains of h679 was converted to h679-Fd-AD1-pdHL2 by replacement of the SacII/EagI fragment with the CH1-AD1 fragment, which was excised from the CH1-AD1-SV3 shuttle vector with SacII and EagI.

Construction of C-DDD1-Fd-hMN-14-pdHL2

C-DDD1-Fd-hMN-14-pdHL2 is an expression vector for production of a stable dimer that comprises two copies of a fusion protein C-DDD1-Fab-hMN-14, in which DDD1 is linked to hMN-14 Fab at the carboxyl terminus of CH1 via a flexible peptide spacer. The plasmid vector hMN-14(I)-pdHL2, which has been used to produce hMN-14 IgG, was converted to C-DDD1-Fd-hMN-14-pdHL2 by digestion with SacII and EagI restriction endonucleases to remove the CH1-CH3 domains and insertion of the CH1-DDD1 fragment, which was excised from the CH1-DDD1-SV3 shuttle vector with SacII and EagI.

The same technique has been utilized to produce plasmids for Fab expression of a wide variety of known antibodies, such as hLL1, hLL2, hPAM4, hR1, hRS7, hMN-14, hMN-15, hA19, hA20 and many others. Generally, the antibody variable region coding sequences were present in a pdHL2 expression vector and the expression vector was converted for production of an AD- or DDD-fusion protein as described above. The AD- and DDD-fusion proteins comprising a Fab fragment of any of such antibodies may be combined, in an approximate ratio of two DDD-fusion proteins per one AD-fusion protein, to generate a trimeric DNL™ construct comprising two Fab fragments of a first antibody and one Fab fragment of a second antibody.

Construction of N-DDD1-Fd-hMN-14-pdHL2

N-DDD1-Fd-hMN-14-pdHL2 is an expression vector for production of a stable dimer that comprises two copies of a fusion protein N-DDD1-Fab-hMN-14, in which DDD1 is linked to hMN-14 Fab at the amino terminus of VH via a flexible peptide spacer. The expression vector was engineered as follows. The DDD1 domain was amplified by PCR.

As a result of the PCR, an NcoI restriction site and the coding sequence for part of the linker containing a BamHI restriction were appended to the 5′ and 3′ ends, respectively. The 170 bp PCR amplimer was cloned into the pGemT vector and clones were screened for inserts in the T7 (5′) orientation. The 194 bp insert was excised from the pGemT vector with NcoI and SalI restriction enzymes and cloned into the SV3 shuttle vector, which was prepared by digestion with those same enzymes, to generate the intermediate vector DDD1-SV3.

The hMN-14 Fd sequence was amplified by PCR. As a result of the PCR, a BamHI restriction site and the coding sequence for part of the linker were appended to the 5′ end of the amplimer. A stop codon and EagI restriction site was appended to the 3′ end. The 1043 bp amplimer was cloned into pGemT. The hMN-14-Fd insert was excised from pGemT with BamHI and EagI restriction enzymes and then ligated with DDD1-SV3 vector, which was prepared by digestion with those same enzymes, to generate the construct N-DDD1-hMN-14Fd-SV3.

The N-DDD1-hMN-14 Fd sequence was excised with XhoI and EagI restriction enzymes and the 1.28 kb insert fragment was ligated with a vector fragment that was prepared by digestion of C-hMN-14-pdHL2 with those same enzymes. The final expression vector was N-DDD1-Fd-hMN-14-pDHL2. The N-linked Fab fragment exhibited similar DNL™ complex formation and antigen binding characteristics as the C-linked Fab fragment (not shown).

C-DDD2-Fd-hMN-14-pdHL2

C-DDD2-Fd-hMN-14-pdHL2 is an expression vector for production of C-DDD2-Fab-hMN-14, which possesses a dimerization and docking domain sequence of DDD2 appended to the carboxyl terminus of the Fd of hMN-14 via a 14 amino acid residue Gly/Ser peptide linker. The fusion protein secreted is composed of two identical copies of hMN-14 Fab held together by non-covalent interaction of the DDD2 domains.

The expression vector was engineered as follows. Two overlapping, complimentary oligonucleotides, which comprise the coding sequence for part of the linker peptide and residues 1-13 of DDD2, were made synthetically. The oligonucleotides were annealed and phosphorylated with T4 PNK, resulting in overhangs on the 5′ and 3′ ends that are compatible for ligation with DNA digested with the restriction endonucleases BamHI and PstI, respectively.

The duplex DNA was ligated with the shuttle vector CH1-DDD1-PGEMT®, which was prepared by digestion with BamHI and PstI, to generate the shuttle vector CH1-DDD2-PGEMT®. A 507 bp fragment was excised from CH1-DDD2-PGEMT® with SacII and EagI and ligated with the IgG expression vector hMN-14(I)-pdHL2, which was prepared by digestion with SacII and EagI. The final expression construct was designated C-DDD2-Fd-hMN-14-pdHL2. Similar techniques have been utilized to generated DDD2-fusion proteins of the Fab fragments of a number of different humanized antibodies.

h679-Fd-AD2-pdHL2

h679-Fab-AD2, was designed to pair as B to C-DDD2-Fab-hMN-14 as A. h679-Fd-AD2-pdHL2 is an expression vector for the production of h679-Fab-AD2, which possesses an anchoring domain sequence of AD2 appended to the carboxyl terminal end of the CH1 domain via a 14 amino acid residue Gly/Ser peptide linker. AD2 has one cysteine residue preceding and another one following the anchor domain sequence of AD1.

The expression vector was engineered as follows. Two overlapping, complimentary oligonucleotides (AD2 Top and AD2 Bottom), which comprise the coding sequence for AD2 and part of the linker sequence, were made synthetically. The oligonucleotides were annealed and phosphorylated with T4 PNK, resulting in overhangs on the 5′ and 3′ ends that are compatible for ligation with DNA digested with the restriction endonucleases BamHI and SpeI, respectively.

The duplex DNA was ligated into the shuttle vector CH1-AD1-PGEMT®, which was prepared by digestion with BamHI and SpeI, to generate the shuttle vector CH1-AD2-PGEMT®. A 429 base pair fragment containing CH1 and AD2 coding sequences was excised from the shuttle vector with SacII and EagI restriction enzymes and ligated into h679-pdHL2 vector that prepared by digestion with those same enzymes. The final expression vector is h679-Fd-AD2-pdHL2.

### Example 6

### Generation of TF1 DNL™ Construct

A large scale preparation of a DNL™ construct, referred to as TF1, was carried out as follows. N-DDD2-Fab-hMN-14 (Protein L-purified) and h679-Fab-AD2 (IMP-291-purified) were first mixed in roughly stoichiometric concentrations in 1 mM EDTA, PBS, pH 7.4. Before the addition of TCEP, SE-HPLC did not show any evidence of a₂b formation (not shown). Instead there were peaks representing a₄ (7.97 min; 200 kDa), a₂ (8.91 min; 100 kDa) and B (10.01 min; 50 kDa). Addition of 5 mM TCEP rapidly resulted in the formation of the a₂b complex as demonstrated by a new peak at 8.43 min, consistent with a 150 kDa protein (not shown). Apparently there was excess B in this experiment as a peak attributed to h679-Fab-AD2 (9.72 min) was still evident yet no apparent peak corresponding to either a₂ or a₄ was observed. After reduction for one hour, the TCEP was removed by overnight dialysis against several changes of PBS. The resulting solution was brought to 10% DMSO and held overnight at room temperature.

When analyzed by SE-HPLC, the peak representing a₂b appeared to be sharper with a slight reduction of the retention time by 0.1 min to 8.31 min (not shown), which, based on our previous findings, indicates an increase in binding affinity. The complex was further purified by IMP-291 affinity chromatography to remove the kappa chain contaminants. As expected, the excess h679-AD2 was co-purified and later removed by preparative SE-HPLC (not shown).

TF1 is a highly stable complex. When TF1 was tested for binding to an HSG (IMP-239) sensorchip, there was no apparent decrease of the observed response at the end of sample injection. In contrast, when a solution containing an equimolar mixture of both C-DDD1-Fab-hMN-14 and h679-Fab-AD1 was tested under similar conditions, the observed increase in response units was accompanied by a detectable drop during and immediately after sample injection, indicating that the initially formed a₂b structure was unstable. Moreover, whereas subsequent injection of WI2 gave a substantial increase in response units for TF1, no increase was evident for the C-DDD1/AD1 mixture.

The additional increase of response units resulting from the binding of WI2 to TF1 immobilized on the sensorchip corresponds to two fully functional binding sites, each contributed by one subunit of N-DDD2-Fab-hMN-14. This was confirmed by the ability of TF1 to bind two Fab fragments of WI2 (not shown). When a mixture containing h679-AD2 and N-DDD1-hMN14, which had been reduced and oxidized exactly as TF1, was analyzed by BIAcore, there was little additional binding of WI2 (not shown), indicating that a disulfide-stabilized a₂b complex such as TF1 could only form through the interaction of DDD2 and AD2.

Two improvements to the process were implemented to reduce the time and efficiency of the process. First, a slight molar excess of N-DDD2-Fab-hMN-14 present as a mixture of a₄/a₂ structures was used to react with h679-Fab-AD2 so that no free h679-Fab-AD2 remained and any a₄/a₂ structures not tethered to h679-Fab-AD2, as well as light chains, would be removed by IMP-291 affinity chromatography. Second, hydrophobic interaction chromatography (HIC) has replaced dialysis or diafiltration as a means to remove TCEP following reduction, which would not only shorten the process time but also add a potential viral removing step. N-DDD2-Fab-hMN-14 and 679-Fab-AD2 were mixed and reduced with 5 mM TCEP for 1 hour at room temperature. The solution was brought to 0.75 M ammonium sulfate and then loaded onto a Butyl PP HIC column. The column was washed with 0.75 M ammonium sulfate, 5 mM EDTA, PBS to remove TCEP. The reduced proteins were eluted from the HIC column with PBS and brought to 10% DMSO. Following incubation at room temperature overnight, highly purified TF1 was isolated by IMP-291 affinity chromatography (not shown). No additional purification steps, such as gel filtration, were required.

### Example 7

### Generation of TF2 DNL™ Construct

A trimeric DNL™ construct designated TF2 was obtained by reacting C-DDD2-Fab-hMN-14 with h679-Fab-AD2. A pilot batch of TF2 was generated with >90% yield as follows. Protein L-purified C-DDD2-Fab-hMN-14 (200 mg) was mixed with h679-Fab-AD2 (60 mg) at a 1.4:1 molar ratio. The total protein concentration was 1.5 mg/ml in PBS containing 1 mM EDTA. Subsequent steps involved TCEP reduction, HIC chromatography, DMSO oxidation, and IMP 291 affinity chromatography. Before the addition of TCEP, SE-HPLC did not show any evidence of a₂b formation. Addition of 5 mM TCEP rapidly resulted in the formation of a₂b complex consistent with a 157 kDa protein expected for the binary structure. TF2 was purified to near homogeneity by IMP 291 affinity chromatography (not shown). IMP 291 is a synthetic peptide containing the HSG hapten to which the 679 Fab binds (Rossi et al., 2005, Clin Cancer Res 11:7122s-29s). SE-HPLC analysis of the IMP 291 unbound fraction demonstrated the removal of a₄, a₂ and free kappa chains from the product (not shown).

The functionality of TF2 was determined by BIACORE® assay. TF2, C-DDD1-hMN-14+h679-AD1 (used as a control sample of noncovalent a₂b complex), or C-DDD2-hMN-14+h679-AD2 (used as a control sample of unreduced a₂ and b components) were diluted to 1 μg/ml (total protein) and passed over a sensorchip immobilized with HSG. The response for TF2 was approximately two-fold that of the two control samples, indicating that only the h679-Fab-AD component in the control samples would bind to and remain on the sensorchip. Subsequent injections of WI2 IgG, an anti-idiotype antibody for hMN-14, demonstrated that only TF2 had a DDD-Fab-hMN-14 component that was tightly associated with h679-Fab-AD as indicated by an additional signal response. The additional increase of response units resulting from the binding of WI2 to TF2 immobilized on the sensorchip corresponded to two fully functional binding sites, each contributed by one subunit of C-DDD2-Fab-hMN-14. This was confirmed by the ability of TF2 to bind two Fab fragments of WI2 (not shown).

### Example 8

### Production of AD- and DDD-Linked Fab and IgG Fusion Proteins From Multiple Antibodies

Using the techniques described in the preceding Examples, the IgG and Fab fusion proteins shown in Table 5 were constructed and incorporated into DNL™ constructs. The fusion proteins retained the antigen-binding characteristics of the parent antibodies and the DNL™ constructs exhibited the antigen-binding activities of the incorporated antibodies or antibody fragments.

TABLE 5
Fusion proteins comprising IgG or Fab

| Fusion Protein | Binding Specificity |
|---|---|
| C-AD1-Fab-h679 | HSG |
| C-AD2-Fab-h679 | HSG |
| C-(AD)₂-Fab-h679 | HSG |
| C-AD2-Fab-h734 | Indium-DTPA |
| C-AD2-Fab-hA20 | CD20 |
| C-AD2-Fab-hA20L | CD20 |
| C-AD2-Fab-hL243 | HLA-DR |
| C-AD2-Fab-hLL2 | CD22 |
| N-AD2-Fab-hLL2 | CD22 |
| C-AD2-IgG-hMN-14 | CEACAM5 |
| C-AD2-IgG-hR1 | IGF-1R |
| C-AD2-IgG-hRS7 | EGP-1 |
| C-AD2-IgG-hPAM4 | MUC |
| C-AD2-IgG-hLL1 | CD74 |
| C-DDD1-Fab-hMN-14 | CEACAM5 |
| C-DDD2-Fab-hMN-14 | CEACAM5 |
| C-DDD2-Fab-h679 | HSG |
| C-DDD2-Fab-hA19 | CD19 |
| C-DDD2-Fab-hA20 | CD20 |
| C-DDD2-Fab-hAFP | AFP |
| C-DDD2-Fab-hL243 | HLA-DR |
| C-DDD2-Fab-hLL1 | CD74 |
| C-DDD2-Fab-hLL2 | CD22 |
| C-DDD2-Fab-hMN-3 | CEACAM6 |
| C-DDD2-Fab-hMN-15 | CEACAM6 |
| C-DDD2-Fab-hPAM4 | MUC |
| C-DDD2-Fab-hR1 | IGF-1R |
| C-DDD2-Fab-hRS7 | EGP-1 |
| N-DDD2-Fab-hMN-14 | CEACAM5 |

### Example 9

### Sequence Variants for DNL™

In addition to the sequences of DDD1, DDD2, DDD3, DDD3C, AD1, AD2 and AD3 described above, other sequence variants of AD and/or DDD moieties may be utilized in construction of the DNL™ complexes. For example, there are only four variants of human PKA DDD sequences, corresponding to the DDD moieties of PKA RIα, RIIα, RIβ and RIIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 54)
SLRECELYVQKHNIQALLKDVSIVQLCTARPERPMAFLREYFEKLEKEE
AK
PKA RIβ
(SEQ ID NO: 55)
SLKGCELYVQLHGIQQVLKDCIVHLCISKPERPMKFLREHFEKLEKEEN
RQILA
PKA RIIα
(SEQ ID NO: 56)
SHIQIPPGLTELLQGYTVEVGQQPPDLVDFAVEYFTRLREARRQ
PKA RIIβ
(SEQ ID NO: 57)
SIEIPAGLTELLQGFTVEVLRHQPADLLEFALQHFTRLQQENER

The structure-function relationships of the AD and DDD domains have been the subject of investigation. (See, e.g., Burns-Hamuro et al., 2005, Protein Sci 14:2982-92; Carr et al., 2001, J Biol Chem 276:17332-38; Alto et al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24:397-408, the entire text of each of which is incorporated herein by reference.)

For example, Kinderman et al. (2006, Mol Cell 24:397-408) examined the crystal structure of the AD-DDD binding interaction and concluded that the human DDD sequence contained a number of conserved amino acid residues that were important in either dimer formation or AKAP binding, underlined in SEQ ID NO:45 below. (See FIG. 1 of Kinderman et al., 2006, incorporated herein by reference.) The skilled artisan will realize that in designing sequence variants of the DDD sequence, one would desirably avoid changing any of the underlined residues, while conservative amino acid substitutions might be made for residues that are less critical for dimerization and AKAP binding.

(SEQ ID NO: 45)
SHIQIPPGLTELLQGYTVEVLRQQPPDLVEFAVEYFTRLREARA

As discussed in more detail below, conservative amino acid substitutions have been characterized for each of the twenty common L-amino acids. Thus, based on the data of Kinderman (2006) and conservative amino acid substitutions, potential alternative DDD sequences based on SEQ ID NO:45 are shown in Table 6. In devising Table 6, only highly conservative amino acid substitutions were considered. For example, charged residues were only substituted for residues of the same charge, residues with small side chains were substituted with residues of similar size, hydroxyl side chains were only substituted with other hydroxyls, etc. Because of the unique effect of proline on amino acid secondary structure, no other residues were substituted for proline. The skilled artisan will realize that a very large number of alternative species within the genus of DDD moieties can be constructed by standard techniques, for example using a commercial peptide synthesizer or well known site-directed mutagenesis techniques. The effect of the amino acid substitutions on AD moiety binding may also be readily determined by standard binding assays, for example as disclosed in Alto et al. (2003, Proc Natl Acad Sci USA 100:4445-50).

TABLE 6
Conservative Amino Acid Substitutions in DDD1 (SEQ ID
NO: 45). Consensus sequence disclosed as SEQ ID NO: 58.

| S | H | I | Q | I | P | P | G | L | T | E | L | L | Q | G | Y | T | V | E | V | L | R |
|---|---|---|---|---|---|---|---|---|---|---|---|---|---|---|---|---|---|---|---|---|---|
| T | K | | N | | | | A | | S | D | | | N | A | | S | | D | | | K |
| | R | | | | | | | | | | | | | | | | | | | | |
| Q | Q | P | P | D | L | V | E | F | A | V | E | Y | F | T | R | L | R | E | A | R | A |
| N | N | | | E | | | D | | L | | D | | | S | K | | K | D | L | K | L |
| | | | | | | | | | I | | | | | | | | | | I | | I |
| | | | | | | | | | V | | | | | | | | | | V | | V |

Alto et al. (2003, Proc Natl Acad Sci USA 100:4445-50) performed a bioinformatic analysis of the AD sequence of various AKAP proteins to design an RII selective AD sequence called AKAP-IS (SEQ ID NO:47), with a binding constant for DDD of 0.4 nM. The AKAP-IS sequence was designed as a peptide antagonist of AKAP binding to PKA. Residues in the AKAP-IS sequence where substitutions tended to decrease binding to DDD are underlined in SEQ ID NO:47 below. The skilled artisan will realize that in designing sequence variants of the AD sequence, one would desirably avoid changing any of the underlined residues, while conservative amino acid substitutions might be made for residues that are less critical for DDD binding. Table 7 shows potential conservative amino acid substitutions in the sequence of AKAP-IS (AD1, SEQ ID NO:47), similar to that shown for DDD1 (SEQ ID NO:45) in Table 6 above.

A large number of AD moiety sequences could be made, tested and used by the skilled artisan, based on the data of Alto et al. (2003). It is noted that FIG. 2 of Alto (2003) shows an even large number of potential amino acid substitutions that may be made, while retaining binding activity to DDD moieties, based on actual binding experiments.

AKAP-IS
(SEQ ID NO: 47)
QIEYLAKQIVDNAIQQA

TABLE 7
Conservative Amino Acid Substitutions in AD1 (SEQ ID NO:
47). Consensus sequence disclosed as SEQ ID NO: 59.

| | | | | | | | | | | | | | | | | |
|---|---|---|---|---|---|---|---|---|---|---|---|---|---|---|---|---|
| Q | I | E | Y | L | A | K | Q | I | V | D | N | A | I | Q | Q | A |
| N | L | D | F | I | | R | N | | | E | Q | | | N | N | L |
| | V | | T | V | | | | | | | | | | | | I |
| | | | S | | | | | | | | | | | | | V |

Gold et al. (2006, Mol Cell 24:383-95) utilized crystallography and peptide screening to develop a SuperAKAP-IS sequence (SEQ ID NO:60), exhibiting a five order of magnitude higher selectivity for the RII isoform of PKA compared with the RI isoform. Underlined residues indicate the positions of amino acid substitutions, relative to the AKAP-IS sequence, which increased binding to the DDD moiety of RIIα. In this sequence, the N-terminal Q residue is numbered as residue number 4 and the C-terminal A residue is residue number 20. Residues where substitutions could be made to affect the affinity for RIM were residues 8, 11, 15, 16, 18, 19 and 20 (Gold et al., 2006). It is contemplated that in certain alternative embodiments, the SuperAKAP-IS sequence may be substituted for the AKAP-IS AD moiety sequence to prepare DNL™ constructs. Other alternative sequences that might be substituted for the AKAP-IS AD sequence are shown in SEQ ID NO:61-63. Substitutions relative to the AKAP-IS sequence are underlined. It is anticipated that, as with the AD2 sequence shown in SEQ ID NO:48, the AD moiety may also include the additional N-terminal residues cysteine and glycine and C-terminal residues glycine and cysteine.

SuperAKAP-IS
(SEQ ID NO: 60)
QIEYVAKQIVDYAIHQA
Alternative AKAP sequences
(SEQ ID NO: 61)
QIEYKAKQIVDHAIHQA
(SEQ ID NO: 62)
QIEYHAKQIVDHAIHQA
(SEQ ID NO: 63)
QIEYVAKQIVDHAIHQA

FIG. 2 of Gold et al. disclosed additional DDD-binding sequences from a variety of AKAP proteins, shown below.

RII-Specific AKAPs
AKAP-KL
(SEQ ID NO: 64)
PLEYQAGLLVQNAIQQAI
AKAP79
(SEQ ID NO: 65)
LLIETASSLVKNAIQLSI
AKAP-Lbc
(SEQ ID NO: 66)
LIEEAASRIVDAVIEQVK
RI-Specific AKAPs
AKAPce
(SEQ ID NO: 67)
ALYQFADRFSELVISEAL
RIAD
(SEQ ID NO: 68)
LEQVANQLADQIIKEAT
PV38
(SEQ ID NO: 69)
FEELAWKIAKMIWSDVF
Dual-Specificity AKAPs
AKAP7
(SEQ ID NO: 70)
ELVRLSKRLVENAVLKAV
MAP2D
(SEQ ID NO: 71)
TAEEVSARIVQVVTAEAV
DAKAP1
(SEQ ID NO: 72)
QIKQAAFQLISQVILEAT
DAKAP2
(SEQ ID NO: 73)
LAWKIAKMIVSDVMQQ

Stokka et al. (2006, Biochem J 400:493-99) also developed peptide competitors of AKAP binding to PKA, shown in SEQ ID NO:74-76. The peptide antagonists were designated as Ht31 (SEQ ID NO:74), RIAD (SEQ ID NO:75) and PV-38 (SEQ ID NO:76). The Ht-31 peptide exhibited a greater affinity for the RII isoform of PKA, while the RIAD and PV-38 showed higher affinity for RI.

Ht31
(SEQ ID NO: 74)
DLIEEAASRIVDAVIEQVKAAGAY
RIAD
(SEQ ID NO: 75)
LEQYANQLADQIIKEATE
PV-38
(SEQ ID NO: 76)
FEELAWKIAKMIWSDVFQQC

Hundsrucker et al. (2006, Biochem J 396:297-306) developed still other peptide competitors for AKAP binding to PKA, with a binding constant as low as 0.4 nM to the DDD of the RII form of PKA. The sequences of various AKAP antagonistic peptides are provided in Table 1 of Hundsrucker et al., reproduced in Table 8 below. AKAPIS represents a synthetic RII subunit-binding peptide. All other peptides are derived from the RII-binding domains of the indicated AKAPs.

TABLE 8
AKAP Peptide sequences

| | Peptide Sequence |
|---|---|
| AKAPIS | QIEYLAKQIVDNAIQQA (SEQ ID NO: 47) |
| AKAPIS-P | QIEYLAKQIPDNAIQQA (SEQ ID NO: 77) |
| Ht31 | KGADLIEEAASRIVDAVIEQVKAAG |
| | (SEQ ID NO: 78) |
| Ht31-P | KGADLIEEAASRIPDAPIEQVKAAG |
| | (SEQ ID NO: 79) |
| AKAP7δ-wt-pep | PEDAELVRLSKRLVENAVLKAVQQY |
| | (SEQ ID NO: 80) |
| AKAP7δ-L304T-pep | PEDAELVRTSKRLVENAVLKAVQQY |
| | (SEQ ID NO: 81) |
| AKAP7δ-L308D-pep | PEDAELVRLSKRDVENAVLKAVQQY |
| | (SEQ ID NO: 82) |
| AKAP7δ-P-pep | PEDAELVRLSKRLPENAVLKAVQQY |
| | (SEQ ID NO: 83) |
| AKAP7δ-PP-pep | PEDAELVRLSKRLPENAPLKAVQQY |
| | (SEQ ID NO: 84) |
| AKAP7δ-L314E-pep | PEDAELVRLSKRLVENAVEKAVQQY |
| | (SEQ ID NO: 85) |
| AKAP1-pep | EEGLDRNEEIKRAAFQIISQVISEA |
| | (SEQ ID NO: 86) |
| AKAP2-pep | LVDDPLEYQAGLLVQNAIQQAIAEQ |
| | (SEQ ID NO: 87) |
| AKAP5-pep | QYETLLIETASSLVKNAIQLSIEQL |
| | (SEQ ID NO: 88) |
| AKAP9-pep | LEKQYQEQLEEEVAKVIVSMSIAFA |
| | (SEQ ID NO: 89) |
| AKAP10-pep | NTDEAQEELAWKIAKMIVSDIMQQA |
| | (SEQ ID NO: 90) |
| AKAP11-pep | VNLDKKAVLAEKIVAEAIEKAEREL |
| | (SEQ ID NO: 91) |
| AKAP12-pep | NGILELETKSSKLVQNIIQTAVDQF |
| | (SEQ ID NO: 92) |
| AKAP14-pep | TQDKNYEDELTQVALALVEDVINYA |
| | (SEQ ID NO: 93) |
| Rab32-pep | ETSAKDNINIEEAARFLVEKILVNH |
| | (SEQ ID NO: 94) |

Residues that were highly conserved among the AD domains of different AKAP proteins are indicated below by underlining with reference to the AKAP IS sequence (SEQ ID NO:47). The residues are the same as observed by Alto et al. (2003), with the addition of the C-terminal alanine residue. (See FIG. 1 of Hundsrucker et al. (2006), incorporated herein by reference.) The sequences of peptide antagonists with particularly high affinities for the RII DDD sequence were those of AKAP-IS, AKAP7δ-wt-pep, AKAP7δ-L304T-pep and AKAP7δ-L308D-pep.

AKAP-IS
(SEQ ID NO: 47)
QIEYLAKQIVDNAIQQA

Carr et al. (2001, J Biol Chem 276:17332-38) examined the degree of sequence homology between different AKAP-binding DDD sequences from human and non-human proteins and identified residues in the DDD sequences that appeared to be the most highly conserved among different DDD moieties. These are indicated below by underlining with reference to the human PKA RIIα DDD sequence of SEQ ID NO:45. Residues that were particularly conserved are further indicated by italics. The residues overlap with, but are not identical to those suggested by Kinderman et al. (2006) to be important for binding to AKAP proteins. The skilled artisan will realize that in designing sequence variants of DDD, it would be most preferred to avoid changing the most conserved residues (italicized), and it would be preferred to also avoid changing the conserved residues (underlined), while conservative amino acid substitutions may be considered for residues that are neither underlined nor italicized.

(SEQ ID NO: 45)
SHIQIPPGLTELLQGYTVEVLRQQPPDLVEFAVEYFTRLREARA

A modified set of conservative amino acid substitutions for the DDD1 (SEQ ID NO:45) sequence, based on the data of Carr et al. (2001) is shown in Table 9. The skilled artisan could readily derive alternative DDD amino acid sequences as disclosed above for Table 6 and Table 7.

TABLE 9
Conservative Amino Acid Substitutions in DDD1 (SEQ ID
NO: 45). Consensus sequence disclosed as SEQ ID NO: 95.

| | | | | | | | | | | | | | | | | | | | | | |
|---|---|---|---|---|---|---|---|---|---|---|---|---|---|---|---|---|---|---|---|---|---|
| S | H | I | Q | [symbol] | [symbol] | P | [symbol] | [symbol] | T | E | [symbol] | [symbol] | Q | [symbol] | [symbol] | [symbol] | V | [symbol] | [symbol] | [symbol] | [symbol] |
| T | | | N | | | | | | S | | | | | | | | I | | | | |
| | | | | | | | | | | | | | | | | | L | | | | |
| | | | | | | | | | | | | | | | | | A | | | | |

| | | | | | | | | | | | | | | | | | | | | | |
|---|---|---|---|---|---|---|---|---|---|---|---|---|---|---|---|---|---|---|---|---|---|
| Q | [symbol] | [symbol] | P | [symbol] | [symbol] | V | E | [symbol] | [symbol] | V | E | [symbol] | [symbol] | T | R | [symbol] | R | E | A | [symbol] | A |
| N | | | | | | | | | | I | D | | | S | K | | K | | L | | L |
| | | | | | | | | | | L | | | | | | | | | I | | I |
| | | | | | | | | | | A | | | | | | | | | V | | V |

The skilled artisan will realize that these and other amino acid substitutions in the DDD or AD amino acid sequences may be utilized to produce alternative species within the genus of AD or DDD moieties, using techniques that are standard in the field and only routine experimentation.

### Example 10

### Antibody-Dendrimer DNL™ Complex for siRNA

Cationic polymers, such as polylysine, polyethylenimine, or polyamidoamine (PAMAM)-based dendrimers, form complexes with nucleic acids. However, their potential applications as non-viral vectors for delivering therapeutic genes or siRNAs remain a challenge. One approach to improve selectivity and potency of a dendrimeric nanoparticle may be achieved by conjugation with an antibody that internalizes upon binding to target cells.

We synthesized and characterized a novel immunoconjugate, designated E1-G5/2, which was made to comprise half of a generation 5 (G5) PAMAM dendrimer (G5/2) site-specifically linked to a stabilized dimer of Fab derived from hRS7, a humanized antibody that is rapidly internalized upon binding to the Trop-2 antigen expressed on various solid cancers.

Methods

E1-G5/2 was prepared by combining two self-assembling modules, AD2-G5/2 and hRS7-Fab-DDD2, under mild redox conditions, followed by purification on a Protein L column. To make AD2-G5/2, we derivatized the AD2 peptide with a maleimide group to react with the single thiol generated from reducing a G5 PAMAM with a cystamine core and used reversed-phase HPLC to isolate AD2-G5/2. We produced hRS7-Fab-DDD2 as a fusion protein in myeloma cells, as described in the Examples above.

The molecular size, purity and composition of E1-G5/2 were analyzed by size-exclusion HPLC, SDS-PAGE, and Western blotting. The biological functions of E1-G5/2 were assessed by binding to an anti-idiotype antibody against hRS7, a gel retardation assay, and a DNase protection assay.

Results

E1-G5/2 was shown by size-exclusion HPLC to consist of a major peak (>90%) flanked by several minor peaks. The three constituents of E1-G5/2 (Fd-DDD2, the light chain, and AD2-G5/2) were detected by reducing SDS-PAGE and confirmed by Western blotting. Anti-idiotype binding analysis revealed E1-G5/2 contained a population of antibody-dendrimer conjugates of different size, all of which were capable of recognizing the anti-idiotype antibody, thus suggesting structural variability in the size of the purchased G5 dendrimer. Gel retardation assays showed E1-G5/2 was able to maximally condense plasmid DNA at a charge ratio of 6:1 (+/−), with the resulting dendriplexes completely protecting the complexed DNA from degradation by DNase I.

Conclusion

The technique can be used to build dendrimer-based nanoparticles that are targetable with antibodies. Such agents have improved properties as carriers of drugs, plasmids or siRNAs for applications in vitro and in vivo. In preferred embodiments, anti-APC and/or anti-DC antibodies, such as anti-HLA-DR, may be utilized to deliver cytotoxic or cytostatic siRNA species to targeted DCs and/or APCs for therapy of organ transplant rejection and other immune dysfunctions.

### Example 11

### Maleimide AD2 Conjugate for Dendrimers

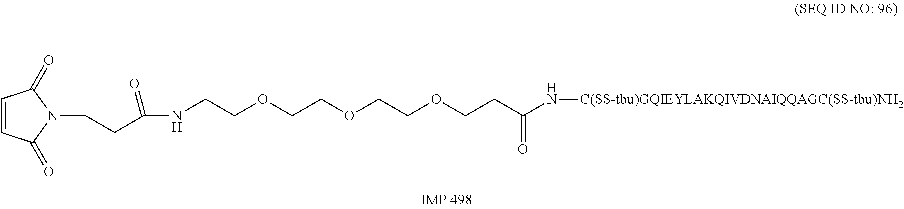

The peptide IMP 498 up to and including the PEG moiety was synthesized on a Protein Technologies PS3 peptide synthesizer by the Fmoc method on Sieber Amide resin (0.1 mmol scale). The maleimide was added manually by mixing the β-maleimidopropionic acid NHS ester with diisopropylethylamine and DMF with the resin for 4 hr. The peptide was cleaved from the resin with 15 mL TFA, 0.5 mL H₂O, 0.5 mL triisopropylsilane, and 0.5 mL thioanisole for 3 hr at room temperature. The peptide was purified by reverse phase HPLC using H₂O/CH₃CN TFA buffers to obtain about 90 mg of purified product after lyophilization.

Synthesis of Reduced G5 Dendrimer (G5/2)

The G-5 dendrimer (10% in MeOH, Dendritic Nanotechnologies), 2.03 g, 7.03×10⁻⁶ mol was reduced with 0.1426 TCEP.HCl 1:1 MeOH/H₂O (˜4 mL) and stirred overnight at room temperature. The reaction mixture was purified by reverse phase HPLC on a C-18 column eluted with 0.1% TFA H₂O/CH₃CN buffers to obtain 0.0633 g of the desired product after lyophilization.

Synthesis of G5/2 Dendrimer-AD2 Conjugate

The G5/2 Dendrimer, 0.0469 g (3.35×10⁻⁶ mol) was mixed with 0.0124 g of IMP 498 (4.4×10⁻⁶ mol) and dissolved in 1:1 MeOH/1M NaHCO₃ and mixed for 19 hr at room temperature followed by treatment with 0.0751 g dithiothreitol and 0.0441 g TCEP.HCl. The solution was mixed overnight at room temperature and purified on a C4 reverse phase HPLC column using 0.1% TFA H₂O/CH₃CN buffers to obtain 0.0033 g of material containing the conjugated AD2 and dendrimer as judged by gel electrophoresis and Western blot.

### Example 12

### Targeted Delivery of siRNA Using Protamine Linked Antibodies

Summary

RNA interference (RNAi) has been shown to down-regulate the expression of various proteins such as HER2, VEGF, Raf-1, bcl-2, EGFR and numerous others in preclinical studies. Despite the potential of RNAi to silence specific genes, the full therapeutic potential of RNAi remains to be realized due to the lack of an effective delivery system to target cells in vivo.

To address this critical need, we developed novel DNL™ constructs having multiple copies of human protamine tethered to a tumor-targeting, internalizing hRS7 (anti-Trop-2) antibody for targeted delivery of siRNAs in vivo. A DDD2-L-thP1 module comprising truncated human protamine (thP1, residues 8 to 29 of human protamine 1) was produced, in which the sequences of DDD2 and thP1 were fused respectively to the N- and C-terminal ends of a humanized antibody light chain (not shown). The sequence of the truncated hP1 (thP1) is shown below. Reaction of DDD2-L-thP1 with the antibody hRS7-IgG-AD2 under mild redox conditions, as described in the Examples above, resulted in the formation of an E1-L-thP1 complex (not shown), comprising four copies of thP1 attached to the carboxyl termini of the hRS7 heavy chains.

tHP1
(SEQ ID NO: 97)
RSQSRSRYYRQRQRSRRRRRRS

The purity and molecular integrity of E1-L-thP1 following Protein A purification were determined by size-exclusion HPLC and SDS-PAGE (not shown). In addition, the ability of E1-L-thP1 to bind plasmid DNA or siRNA was demonstrated by the gel shift assay (not shown). E1-L-thP1 was effective at binding short double-stranded oligonucleotides (not shown) and in protecting bound DNA from digestion by nucleases added to the sample or present in serum (not shown).

The ability of the E1-L-thP1 construct to internalize siRNAs into Trop-2-expressing cancer cells was confirmed by fluorescence microscopy using FITC-conjugated siRNA and the human Calu-3 lung cancer cell line (not shown).

Methods

The hRS7 IgG-AD module, constructed as described in the Examples above, was expressed in myeloma cells and purified from the culture supernatant using Protein A affinity chromatography. The DDD2-L-thP1 module was expressed as a fusion protein in myeloma cells and was purified by Protein L affinity chromatography. Since the CH3-AD2-IgG module possesses two AD2 peptides and each can bind to a DDD2 dimer, with each DDD2 monomer attached to a protamine moiety, the resulting E1-L-thP1 conjugate comprises four protamine groups. E1-L-thp1 was formed in nearly quantitative yield from the constituent modules and was purified to near homogeneity (not shown) with Protein A.

DDD2-L-thP1 was purified using Protein L affinity chromatography and assessed by size exclusion HPLC analysis and SDS-PAGE under reducing and nonreducing conditions (data not shown). A major peak was observed at 9.6 min (not shown). SDS-PAGE showed a major band between 30 and 40 kDa in reducing gel and a major band about 60 kDa (indicating a dimeric form of DDD2-L-thP1) in nonreducing gel (not shown). The results of Western blotting confirmed the presence of monomeric DDD2-L-tP1 and dimeric DDD2-L-tP1 on probing with anti-DDD antibodies (not shown).

To prepare the E1-L-thP1, hRS7-IgG-AD2 and DDD2-L-thP1 were combined in approximately equal amounts and reduced glutathione (final concentration 1 mM) was added. Following an overnight incubation at room temperature, oxidized glutathione was added (final concentration 2 mM) and the incubation continued for another 24 h. E1-L-thP1 was purified from the reaction mixture by Protein A column chromatography and eluted with 0.1 M sodium citrate buffer (pH 3.5). The product peak was neutralized, concentrated, dialyzed with PBS, filtered, and stored in PBS containing 5% glycerol at 2 to 8° C. The composition of E1-L-thP1 was confirmed by reducing SDS-PAGE (not shown), which showed the presence of all three constituents (AD2-appended heavy chain, DDD2-L-htP1, and light chain).

The ability of DDD2-L-thP1 (not shown) and E1-L-thP1 (not shown) to bind DNA was evaluated by gel shift assay. DDD2-L-thP1 retarded the mobility of 500 ng of a linear form of 3-kb DNA fragment in 1% agarose at a molar ratio of 6 or higher (not shown). E1-L-thP1 retarded the mobility of 250 ng of a linear 200-bp DNA duplex in 2% agarose at a molar ratio of 4 or higher (not shown), whereas no such effect was observed for hRS7-IgG-AD2 alone (not shown). The ability of E1-L-thP1 to protect bound DNA from degradation by exogenous DNase and serum nucleases was also demonstrated (not shown).

The ability of E1-L-thP1 to promote internalization of bound siRNA was examined in the Trop-2 expressing ME-180 cervical cell line (not shown). Internalization of the E1-L-thP1 complex was monitored using FITC conjugated goat anti-human antibodies. The cells alone showed no fluorescence (not shown). Addition of FITC-labeled siRNA alone resulted in minimal internalization of the siRNA (not shown). Internalization of E1-L-thP1 alone was observed in 60 minutes at 37° C. (not shown). E1-L-thP1 was able to effectively promote internalization of bound FITC-conjugated siRNA (not shown). E1-L-thP1 (10 μg) was mixed with FITC-siRNA (300 nM) and allowed to form E1-L-thP1-siRNA complexes which were then added to Trop-2-expressing Calu-3 cells. After incubation for 4 h at 37° C. the cells were checked for internalization of siRNA by fluorescence microscopy (not shown).

The ability of E1-L-thP1 to induce apoptosis by internalization of siRNA was examined. E1-L-thP1 (10 μg) was mixed with varying amounts of siRNA (AllStars Cell Death siRNA, Qiagen, Valencia, Calif.). The E1-L-thP1-siRNA complex was added to ME-180 cells. After 72 h of incubation, cells were trypsinized and annexin V staining was performed to evaluate apoptosis. The Cell Death siRNA alone or E1-L-thP1 alone had no effect on apoptosis (not shown). Addition of increasing amounts of E1-L-thP1-siRNA produced a dose-dependent increase in apoptosis (not shown). These results show that E1-L-thP1 could effectively deliver siRNA molecules into the cells and induce apoptosis of target cells.

Conclusions

The technology used to make DNL™ complexes provides a modular approach to efficiently tether multiple protamine molecules to the anti-Trop-2 hRS7 antibody resulting in the novel molecule E1-L-thP1. SDS-PAGE demonstrated the homogeneity and purity of E1-L-thP1. DNase protection and gel shift assays showed the DNA binding activity of E1-L-thP1. E1-L-thP1 internalized in the cells like the parental hRS7 antibody and was able to effectively internalize siRNA molecules into Trop-2-expressing cells, such as ME-180 and Calu-3.

The skilled artisan will realize that the technique is not limited to any specific antibody or siRNA species. Rather, the same methods and compositions demonstrated herein can be used to make targeted delivery complexes comprising any antibody, any siRNA carrier and any siRNA species. The use of a bivalent IgG in targeted delivery complexes would result in prolonged circulating half-life and higher binding avidity to target cells, resulting in increased uptake and improved efficacy.

### Example 13

### Hexavalent DNL™ Constructs

The technology described above for formation of trivalent DNL™ complexes was applied to generate hexavalent IgG-based DNL™ structures (HIDS). Because of the increased number of binding sites for target antigens, hexavalent constructs might be expected to show greater affinity and/or efficacy against target cells. Two types of modules, which were produced as recombinant fusion proteins, were combined to generate a variety of HIDS. Fab-DDD2 modules were as described for use in generating trivalent Fab structures (Rossi et al. Proc Natl Acad Sci USA. 2006; 103(18): 6841-6). The Fab-DDD2 modules form stable homodimers that bind to AD2-containing modules. To generate HIDS, two types of IgG-AD2 modules were created to pair with the Fab-DDD2 modules: C-H-AD2-IgG and N-L-AD2-IgG.

C-H-AD2-IgG modules have an AD2 peptide fused to the carboxyl terminus (C) of the heavy (H) chain of IgG via a 9 amino acid residue peptide linker. The DNA coding sequences for the linker peptide followed by the AD2 peptide are coupled to the 3′ end of the CH3 (heavy chain constant domain 3) coding sequence by standard recombinant DNA methodologies, resulting in a contiguous open reading frame. When the heavy chain-AD2 polypeptide is co-expressed with a light chain polypeptide, an IgG molecule is formed possessing two AD2 peptides, which can therefore bind two Fab-DDD2 dimers. The C-H-AD2-IgG module can be combined with any Fab-DDD2 module to generate a wide variety of hexavalent structures composed of an Fc fragment and six Fab fragments. If the C-H-AD2-IgG module and the Fab-DDD2 module are derived from the same parental monoclonal antibody (MAb) the resulting HIDS is monospecific with 6 binding arms to the same antigen. If the modules are instead derived from two different MAbs then the resulting HIDS are bispecific, with two binding arms for the specificity of the C-H-AD2-IgG module and 4 binding arms for the specificity of the Fab-DDD2 module.

N-L-AD2-IgG is an alternative type of IgG-AD2 module in which an AD2 peptide is fused to the amino terminus (N) of the light (L) chain of IgG via a peptide linker. The L chain can be either Kappa (K) or Lambda (λ) and will also be represented as K. The DNA coding sequences for the AD2 peptide followed by the linker peptide are coupled to the 5′ end of the coding sequence for the variable domain of the L chain (V_{L}), resulting in a contiguous open reading frame. When the AD2-kappa chain polypeptide is co-expressed with a heavy chain polypeptide, an IgG molecule is formed possessing two AD2 peptides, which can therefore bind two Fab-DDD2 dimers. The N-L-AD2-IgG module can be combined with any Fab-DDD2 module to generate a wide variety of hexavalent structures composed of an Fc fragment and six Fab fragments.

The same technique has been utilized to produce DNL™ complexes comprising an IgG moiety attached to four effector moieties, such as cytokines. In an exemplary embodiment, an IgG moiety was attached to four copies of interferon-α2b. The antibody-cytokine DNL™ construct exhibited superior pharmacokinetic properties and/or efficacy compared to PEGylated forms of interferon-α2b.

### Example 14

### Generation of Hexavalent DNL™ Constructs

Generation of Hex-hA20

The method used to make DNL™ complexes was employed to create Hex-hA20, a monospecific anti-CD20 HIDS, by combining C-H-AD2-hA20 IgG with hA20-Fab-DDD2. The Hex-hA20 structure contains six anti-CD20 Fab fragments and an Fc fragment, arranged as four Fab fragments and one IgG antibody. Hex-hA20 was made in four steps.

Step 1, Combination:

A 210% molar equivalent of (hA20-Fab-DDD2)₂ was mixed with C-H-AD2-hA20 IgG. This molar ratio was used because two Fab-DDD2 dimers are coupled to each C-H-AD2-hA20 IgG molecule and an additional 10% excess of the former ensures that the coupling reaction is complete. The molecular weights of C-H-AD2-hA20 IgG and (hA20-Fab-DDD2)₂ are 168 kDa and 107 kDa, respectively. As an example, 134 mg of hA20-Fab-DDD2 would be mixed with 100 mg of C-H-AD2-hA20 IgG to achieve a 210% molar equivalent of the former. The mixture is typically made in phosphate buffered saline, pH 7.4 (PBS) with 1 mM EDTA.

Step 2, Mild Reduction:

Reduced glutathione (GSH) was added to a final concentration of 1 mM and the solution is held at room temperature (16-25° C.) for 1-24 hours.

Step 3, Mild Oxidation:

Following reduction, oxidized glutathione (GSSH) was added directly to the reaction mixture to a final concentration of 2 mM and the solution was held at room temperature for 1-24 hours.

Step 4, Isolation of the DNL™ Product:

Following oxidation, the reaction mixture was loaded directly onto a Protein-A affinity chromatography column. The column was washed with PBS and the Hex-hA20 was eluted with 0.1 M glycine, pH 2.5. Since excess hA20-Fab-DDD2 was used in the reaction, there was no unconjugated C-H-AD2-hA20 IgG, or incomplete DNL™ structures containing only one (hA20-Fab-DDD2)₂ moiety. The unconjugated excess hA20-Fab-DDD2 does not bind to the affinity resin. Therefore, the Protein A-purified material contains only the desired product.

The calculated molecular weight from the deduced amino acid sequences of the constituent polypeptides is 386 kDa. Size exclusion HPLC analysis showed a single protein peak with a retention time consistent with a protein structure of 375-400 kDa (not shown). SDS-PAGE analysis under non-reducing conditions showed a cluster of high molecular weight bands indicating a large covalent structure (not shown). SDS-PAGE under reducing conditions showed the presence of only the three expected polypeptide chains: the AD2-fused heavy chain (HC-AD2), the DDD2-fused Fd chain (Fd-DDD2), and the kappa chains (not shown).

Generation of Hex-hLL2

The same method was used to create a monospecific anti-CD22 HIDS (Hex-hLL2) by combining C-H-AD2-hLL2 IgG with hLL2-Fab-DDD2. The reaction was accomplished as described above for Hex-hA20. The calculated molecular weight from the deduced amino acid sequences of the constituent polypeptides is 386 kDa. Size exclusion HPLC analysis showed a single protein peak with a retention time consistent with a protein structure of 375-400 kDa (not shown). SDS-PAGE analysis under non-reducing conditions showed a cluster of high molecular weight bands, which were eliminated under reducing conditions to leave only the three expected polypeptide chains: HC-AD2, Fd-DDD2, and the kappa chain (not shown).

The following hexavalent DNL™ complexes have been produced using the same methods discussed above. In all cases, the hexavalent DNL™ complexes retained the binding activities of the parent antibodies or antibody fragments. The hexavalent DNL™ complexes were stable in serum under physiological conditions.

TABLE 10
Hexavalent DNL ™ Constructs

| Designation | AD-module | Antigen | Valency | DDD-module | Antigen | Valency |
|---|---|---|---|---|---|---|
| C2-C2-C2 | c-AD2-IgG-h243 | HLA-DR | 2 | c-DDD2-Fab-hL243 | HLA-DR | 4 |
| 20-20-20 | c-AD2-IgG-hA20 | CD20 | 2 | c-DDD2-Fab-hA20 | CD20 | 4 |
| | c-AD2-IgG-hA20(L) | | | | | |
| 22-22-22 | c-AD2-IgG-hLL2 | CD22 | 2 | c-DDD2-Fab-hLL2 | CD22 | 4 |
| | c-AD2-IgG-hLL1 | CD74 | 2 | | | |
| 1R-1R-1R | c-AD2-IgG-hR1 | IGF-1R | 2 | c-DDD2-Fab-hR1 | IGF-IR | 4 |
| E1-E1-E1 | c-AD2-IgG-hRS7 | TROP-2 | 2 | c-DDD2-Fab-hRS7 | TROP-2 | 4 |
| M1-M1-M1 | c-AD2-IgG-hPAM4 | MUC1 | 2 | c-DDD2-Fab-hPAM4 | MUC1 | 4 |
| 14-14-14 | c-AD2-IgG-hMN-14 | CEACAM5 | 2 | c-DDD2-Fab-hMN-14 | CEACAM5 | 4 |
| | c-AD2-IgG-h734 | In-DTPA | | | | |
| | c-AD2-IgG-h734(L) | | | | | |
| 20-C2-C2 | c-AD2-IgG-hA20 | CD20 | 2 | c-DDD2-Fab-hL243 | HLA-DR | 4 |
| 20-19-19 | c-AD2-IgG-hA20 | CD20 | 2 | c-DDD2-Fab-hA19 | CD19 | 4 |
| C2-19-19 | c-AD2-IgG-h243 | HLA-DR | 2 | c-DDD2-Fab-hA19 | CD19 | 4 |
| C2-14-14 | c-AD2-IgG-h243 | HLA-DR | 2 | c-DDD2-Fab-hMN-14 | CEACAM5 | 4 |
| C2-74-74 | c-AD2-IgG-h243 | HLA-DR | 2 | c-DDD2-Fab-hLLl | CD74 | 4 |
| C2-22-22 | c-AD2-IgG-h243 | HLA-DR | 2 | c-DDD2-Fab-hLL2 | CD22 | 4 |
| C2-20-20 | c-AD2-IgG-h243 | HLA-DR | 2 | c-DDD2-Fab-hA20 | CD22 | 4 |
| 20-74-74 | c-AD2-IgG-hA20 | CD20 | 2 | c-DDD2-Fab-hLLL1 | CD74 | 4 |
| 20-22-22 | c-AD2-IgG-hA20 | CD20 | 2 | c-DDD2-Fab-hLLL2 | CD22 | 4 |
| 20-14-14 | c-AD2-IgG-hA20 | CD20 | 2 | c-DDD2-Fab-hMN-14 | CEACAM5 | 4 |
| 22-20-20 | c-AD2-IgG-hLL2 | CD22 | 2 | c-DDD2-Fab-hA20 | CD20 | 4 |
| 22-14-14 | c-AD2-IgG-hLL2 | CD22 | 2 | c-DDD2-Fab-hMN-14 | CEACAM5 | 4 |
| 74-20-20 | c-AD2-IgG-hLL1 | CD74 | 2 | c-DDD2-Fab-hA20 | CD20 | 4 |
| 14-(679)4 | c-AD2-IgG-hMN-14 | CEACAM5 | 2 | c-DDD2-Fab-h679 | HSG | 4 |

### Example 15

### Ribonuclease Based DNL™ Immunotoxins Comprising Quadruple Ranpirnase (Rap) Conjugated to B-Cell Targeting Antibodies

We applied the DNL™ method to generate a novel class of immunotoxins, each of which comprises four copies of Rap site-specifically linked to a bivalent IgG. We combined a recombinant Rap-DDD module, produced in E. coli, with recombinant, humanized IgG-AD modules, which were produced in myeloma cells and targeted B-cell lymphomas and leukemias via binding to CD20 (hA20, veltuzumab), CD22 (hLL2, epratuzumab) or HLA-DR (hL243, IMMU-114), to generate 20-Rap, 22-Rap and C2-Rap, respectively. For each construct, a dimer of Rap was covalently tethered to the C-terminus of each heavy chain of the respective IgG. A control construct, 14-Rap, was made similarly, using labetuzumab (hMN-14), that binds to an antigen (CEACAM5) not expressed on B-cell lymphomas/leukemias.

Rap-DDD2
(SEQ ID NO: 99)
pQDWLTFQKKHITNTRDVDCDNIMSTNLFHCKDKNTFIYSRPEPVKAICKGIIASKNVLT
TSEFYLSDCNVTSRPCKYKLKKSTNKFCVTCENQAPVHFVGVGSCGGGGSLECGHIQIP
PGLTELLQGYTVEVLRQQPPDLVEFAVEYFTRLREARAVEHHHHHH

The deduced amino acid sequence of secreted Rap-DDD2 is shown above (SEQ ID NO:99). Rap, underlined; linker, italics; DDD2, bold; pQ, amino-terminal glutamine converted to pyroglutamate. Rap-DDD2 was produced in E. coli as inclusion bodies, which were purified by IMAC under denaturing conditions, refolded and then dialyzed into PBS before purification by Q-Sepharose anion exchange chromatography. SDS-PAGE under reducing conditions resolved a protein band with a Mr appropriate for Rap-DDD2 (18.6 kDa) (not shown). The final yield of purified Rap-DDD2 was 10 mg/L of culture.

The DNL™ method was employed to rapidly generate a panel of IgG-Rap conjugates. The IgG-AD modules were expressed in myeloma cells and purified from the culture supernatant using Protein A affinity chromatography. The Rap-DDD2 module was produced and mixed with IgG-AD2 to form a DNL™ complex. Since the CH3-AD2-IgG modules possess two AD2 peptides and each can tether a Rap dimer, the resulting IgG-Rap DNL™ construct comprises four Rap groups and one IgG. IgG-Rap is formed nearly quantitatively from the constituent modules and purified to near homogeneity with Protein A.

Prior to the DNL™ reaction, the CH3-AD2-IgG exists as both a monomer, and a disulfide-linked dimer (not shown). Under non-reducing conditions, the IgG-Rap resolves as a cluster of high molecular weight bands of the expected size between those for monomeric and dimeric CH3-AD2-IgG (not shown). Reducing conditions, which reduces the conjugates to their constituent polypeptides, shows the purity of the IgG-Rap and the consistency of the DNL™ method, as only bands representing heavy-chain-AD2 (HC-AD2), kappa light chain and Rap-DDD2 were visualized (not shown).

Reversed phase HPLC analysis of 22-Rap (not shown) resolved a single protein peak at 9.10 min eluting between the two peaks of CH3-AD2-IgG-hLL2, representing the monomeric (7.55 min) and the dimeric (8.00 min) forms. The Rap-DDD2 module was isolated as a mixture of dimer and tetramer (reduced to dimer during DNL™), which were eluted at 9.30 and 9.55 min, respectively (not shown).

LC/MS analysis of 22-Rap was accomplished by coupling reversed phase HPLC using a C8 column with ESI-TOF mass spectrometry (not shown). The spectrum of unmodified 22-Rap identifies two major species, having either two GOF (GOF/GOF) or one GOF plus one G1F (GOF/G1F) N-linked glycans, in addition to some minor glycoforms (not shown). Enzymatic deglycosylation resulted in a single deconvoluted mass consistent with the calculated mass of 22-Rap (not shown). The resulting spectrum following reduction with TCEP identified the heavy chain-AD2 polypeptide modified with an N-linked glycan of the GOF or G1F structure as well as additional minor forms (not shown). Each of the three subunit polypeptides comprising 22-Rap were identified in the deconvoluted spectrum of the reduced and deglycosylated sample (not shown). The results confirm that both the Rap-DDD2 and HC-AD2 polypeptides have an amino terminal glutamine that is converted to pyroglutamate (pQ); therefore, 22-Rap has 6 of its 8 constituent polypeptides modified by pQ.

In vitro cytotoxicity was evaluated in three NHL cell lines. Each cell line expresses CD20 at a considerably higher surface density compared to CD22; however, the internalization rate for hLL2 (anti-CD22) is much faster than hA20 (anti-CD20). 14-Rap shares the same structure as 22-Rap and 20-Rap, but its antigen (CEACAM5) is not expressed by the NHL cells. Cells were treated continuously with IgG-Rap as single agents or with combinations of the parental MAbs plus rRap. Both 20-Rap and 22-Rap killed each cell line at concentrations above 1 nM, indicating that their action is cytotoxic as opposed to merely cytostatic (not shown). 20-Rap was the most potent IgG-Rap, suggesting that antigen density may be more important than internalization rate. Similar results were obtained for Daudi and Ramos, where 20-Rap (EC50˜0.1 nM) was 3-6-fold more potent than 22-Rap (not shown). The rituximab-resistant mantle cell lymphoma line, Jeko-1, exhibits increased CD20 but decreased CD22, compared to Daudi and Ramos. Importantly, 20-Rap exhibited very potent cytotoxicity (EC₅₀˜20 pM) in Jeko-1, which was 25-fold more potent than 22-Rap (not shown).

The DNL™ method provides a modular approach to efficiently tether multiple cytotoxins onto a targeting antibody, resulting in novel immunotoxins that are expected to show higher in vivo potency due to improved pharmacokinetics and targeting specificity. LC/MS, RP-HPLC and SDS-PAGE demonstrated the homogeneity and purity of IgG-Rap. Targeting Rap with a MAb to a cell surface antigen enhanced its tumor-specific cytotoxicity. Antigen density and internalization rate are both critical factors for the observed in vitro potency of IgG-Rap. In vitro results show that CD20-, CD22-, or HLA-DR-targeted IgG-Rap have potent biologic activity for therapy of B-cell lymphomas and leukemias.

It will be readily apparent to one skilled in the art that varying substitutions and modifications may be made to the invention disclosed herein without departing from the scope and spirit of the invention. Thus, such additional embodiments are within the scope of the present invention.

TABLE 3
Summary of phenotypic changes of PBMCs

| | MLR/ | MLR/ | | Resting PBMCs/ | Resting PBMCs/ | |
|---|---|---|---|---|---|---|
| | Control Ab | IMMU-114 | P-value¹⁾ | Control Ab (%) | IMMU-114 (%) | P-value²⁾ |
| CD3+ | 86.3 ± 0.8 | 90.7 ± 2.2 | 0.059 | 82.7 ± 2.5 | 87.8 ± 1.1 | 0.055 |
| CD16+CD56+ | 8.3 ± 0.3 | 3.7 ± 0.2 | 0.004 | 18.5 ± 0.7 | 7.7 ± 0.5 | 0.004 |
| CD3+CD16+CD56 | 0.6 ± 0.1 | 0.3 ± 0.1 | 0.205 | 1.2 ± 0.1 | 0.8 ± 0.1 | 0.003 |
| CD4+ | 53.0 ± 0.5 | 60.8 ± 1.1 | 0.035 | 53.7 ± 1.9 | 58.2 ± 2.5 | 0.144 |
| CD8+ | 32.7 ± 1.5 | 31.0 ± 2.0 | 0.319 | 35.2 ± 1.6 | 33.1 ± 2.1 | 0.223 |
| CD19+ | 3.1 ± 0.1 | 0.6 ± 0.1 | <0.001 | 3.4 ± 0.2 | 1.0 ± 0.2 | <0.001 |
| CD3+CD25 high+ | 5.6 ± 0.6 | 3.9 ± 0.3 | 0.023 | 5.2 ± 0.2 | 1.5 ± 0.3 | <0.001 |
| CD3+class II DR+ | 52.4 ± 2.5 | 5.2 ± 1.5 | <0.001 | 34.3 ± 1.6 | 0.6 ± 0.2 | <0.001 |
| CD14+class II DR+ | 4.8 ± 0.4 | 2.5 ± 0.6 | 0.008 | 1.8 ± 0.2 | 0.6 ± 0.2 | 0.002 |
| CD11c+class II DR | 15.5 ± 1.4 | 2.5 ± 0.6 | 0.001 | 5.5 ± 0.5 | 0.5 ± 0.2 | <0.001 |

What is claimed is:

1. A method of suppressing allogeneic immune response to organ transplantation comprising administering an anti-HLA-DR antibody or antigen-binding fragment thereof to an organ transplant recipient.

2. The method of claim 1, wherein the anti-HLA-DR antibody is a naked antibody.

3. The method of claim 2, further comprising administering at least one therapeutic agent to the organ transplant recipient.

4. The method of claim 4, wherein the therapeutic agent is selected from the group consisting of a steroid, an alkylating agent, an antimetabolite, a cytokine, an antibody, an antibody fragment, a drug, a cytotoxin, an immunomodulator, a pro-apoptotic agent, an siRNA and an RNAi.

5. The method of claim 1, wherein the anti-HLA-DR antibody is conjugated to at least one therapeutic agent.

6. The method of claim 4, wherein the therapeutic agent is selected from the group consisting of a steroid, an alkylating agent, an antimetabolite, a cytokine, an antibody, an antibody fragment, a drug, a radionuclide, a cytotoxin, an immunomodulator, a pro-apoptotic agent, an siRNA and an RNAi.

7. The method of claim 6, wherein the therapeutic agent is selected from the group consisting of 10-hydroxycamptothecin, 3′,5′-O-dioleoyl-FudR, 6-mercaptopurine, abrin, anastrozole, anthracycline, aplidin, azacytidine, azaribine, azathiopurine, basiliximab, bleomycin, bleomycin, bortezomib, bryostatin-1, busulfan, calicheamycin, camptothecin, carboplatin, carmustine, celebrex, chlorambucil, cisplatin, cladribine, cyclophosphamide, cyclosporin, cytarabine, dacarbazine, daclizumab, dactinomycin, daunomycin glucuronide, daunorubicin, dexamethasone, diethylstilbestrol, diphtheria toxin, DNase I, docetaxel, doxorubicin glucuronide, doxorubicin, epirubicin glucuronide, estramustine, ethinyl estradiol, etoposide, etoposide phosphate, fingolimod, floxuridine, fludarabine, fluorouracil, fluoxymesterone, flutamide, gelonin, gemcitabine, hydroxyprogesterone caproate, hydroxyurea, idarubicin, ifosfamide, irinotecan (CPT-11), L-asparaginase, leucovorin, lomustine, mechlorethamine, medroprogesterone acetate, megestrol acetate, melphalan, mercaptopurine, methotrexate, mithramycin, mitomycin C, mitomycin, mitotane, mitoxantrone, muromonab, mycophenolate, myriocin, onconase, paclitaxel, pentostatin, phenyl butyrate, pokeweed antiviral protein, prednisolone, prednisone, procarbazine, Pseudomonas endotoxin, Pseudomonas exotoxin, PSI-341, rapLR1, ribonuclease, ricin, semustine, sirolimus, SN-38, Staphylococcal enterotoxin-A, streptozocin, tacrolimus, tamoxifen, taxanes, taxol, teniposide, testosterone propionate, thalidomide, thioguanine, thiotepa, topotecan, uracil mustard, velcade, vinblastine, vincristine and vinorelbine.

8. The method of claim 6, wherein the antibody or antibody fragment binds to an antigen selected from the group consisting of IL-1, IL-2, IL-2R, IL-3, IL-4, IL-5, IL-6, IL-8, TNFα, CD3, CD19, CD20, CD22, CD25, CD74.

9. The method of claim 6, wherein the radionuclide is selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

10. The method of claim 6, wherein the immunomodulator is selected from the group consisting of erythropoietin, thrombopoietin tumor necrosis factor-α (TNF), TNF-β, granulocyte-colony stimulating factor (G-CSF), granulocyte macrophage-colony stimulating factor (GM-CSF), interferon-α, interferon-β, interferon-γ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin and lymphotoxin.

11. The method of claim 1, wherein the anti-HLA-DR antibody or fragment thereof is administered before, concurrently with or after organ transplantation.

12. The method of claim 1, further comprising depleting mDCs, pDCs, B cells and monocytes, without depleting T cells.

13. The method of claim 1, further comprising suppressing proliferation of allo-reactive T cells, without suppressing cytomegalovirus (CMV)-specific, CD8⁺ memory T cells.

14. The method of claim 1, further comprising killing activated but not resting HLA-DR⁺ T cells.

15. The method of claim 1, further comprising inhibiting production of Th1 cytokines IL-2, IFN-γ and TNF-α

16. A method of suppressing xenogeneic immune response to organ transplantation comprising administering an anti-HLA-DR antibody or antigen-binding fragment thereof to an organ transplant recipient, wherein the organ transplant recipient is of a different species from the organ transplant donor.

17. The method of claim 16, wherein the anti-HLA-DR antibody is a naked antibody.

18. The method of claim 16, further comprising administering at least one therapeutic agent to the organ transplant recipient.

19. The method of claim 18, wherein the therapeutic agent is selected from the group consisting of a steroid, an alkylating agent, an antimetabolite, a cytokine, an antibody, an antibody fragment, a drug, a cytotoxin, an immunomodulator, a pro-apoptotic agent, an siRNA and an RNAi.

20. The method of claim 16, wherein the anti-HLA-DR antibody is conjugated to at least one therapeutic agent.

21. The method of claim 20, wherein the therapeutic agent is selected from the group consisting of a steroid, an alkylating agent, an antimetabolite, a cytokine, an antibody, an antibody fragment, a drug, a radionuclide, a cytotoxin, an immunomodulator, a pro-apoptotic agent, an siRNA and an RNAi.

22. The method of claim 21, wherein the therapeutic agent is selected from the group consisting of cyclosporin, tacrolimus, sirolimus, cyclophosphamide, methotrexate, azathiopurine, mercaptopurine, dactinomycin, anthracycline, mitomycin C, bleomycin mithramycin, fingolimod, myriocin, prednisolone, mycophenolate, basiliximab, daclizumab, muromonab, aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin

23. The method of claim 21, wherein the antibody or antibody fragment binds to an antigen selected from the group consisting of IL-1, IL-2, IL-2R, IL-3, IL-4, IL-5, IL-6, IL-8, TNFα, CD3, CD19, CD20, CD22, CD25, CD74.

24. The method of claim 21, wherein the therapeutic agent is a radionuclide selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

25. The method of claim 21, wherein the therapeutic agent is an immunomodulator selected from the group consisting of erythropoietin, thrombopoietin tumor necrosis factor-α (TNF), TNF-β, granulocyte-colony stimulating factor (G-CSF), granulocyte macrophage-colony stimulating factor (GM-CSF), interferon-α, interferon-β, interferon-γ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-B, platelet-growth factor, TGF-α, TGF-β, insulin-like growth factor-I, insulin-like growth factor-II, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin and lymphotoxin.

26. The method of claim 16, wherein the anti-HLA-DR antibody or fragment thereof is administered before, concurrently with or after organ transplantation.

27. The method of claim 16, further comprising depleting mDCs, pDCs, B cells and monocytes, without depleting T cells.

28. The method of claim 16, further comprising suppressing proliferation of allo-reactive T cells, without suppressing cytomegalovirus (CMV)-specific, CD8⁺ memory T cells.

29. The method of claim 16, further comprising suppressing production of IL-2, IFN-γ, TNF-α, and IL-6.

30. The method of claim 16, further comprising suppressing xenogeneic immune response through both direct inhibition of the interaction between MHC class II HLA-DR-positive cells and CD4+ T cells, and indirect suppression of proinflammatory cytokine production.
